(19) United States
(12) Patent Application Publication
(10) Pub. No.: US 2023/0181638 A1
(43) Pub. Date: Jun. 15, 2023

(54) IMMUNOGENIC PEPTIDES WITH NEW OXIDOREDUCTASE MOTIFS

(71) Applicant: IMCYSE SA, Angleur (Liège) (BE)

(72) Inventor: Milos ERAK, Bruxelles (BE)

(21) Appl. No.: 17/923,217

(22) PCT Filed: May 6, 2021
(86) PCT No.: PCT/EP2021/062024

§ 371 Date: Nov. 3, 2022

(30) Foreign Application Priority Data
May 6, 2020 (EP) 20173223.7

(51) Int. Cl. A61K 35/17; C07K 14/74; C07K 14/00; C12N 5/0783; A61P 37/06

(52) U.S. Cl. CPC A61K 35/17; C07K 14/70539; C07K 14/001; C12N 5/0638; C12N 5/0646; A61P 37/06

(57) ABSTRACT

The invention relates to immunogenic peptides comprising T-cell epitopes and new oxidoreductase motifs with increased activity, and their use in the treatment and/or prevention of type-1 diabetes (T1D), multiple sclerosis (MS), neuromyelitis optica (NMO), or rheumatoid arthritis (RA) in subjects.

### FIELD OF THE INVENTION

The present invention relates to immunogenic peptides. In particular, the invention relates to immunogenic peptides comprising an oxidoreductase motif linked to a T cell epitope derived from auto-antigens or allergens. More particularly, the invention pertains to the improvement of the oxidoreductase motif in order to create more potent peptides for use in therapy of diseases related to unwanted immune response against an auto-antigen or allergen.

### BACKGROUND OF THE INVENTION

Several strategies have been described to prevent the generation of an unwanted immune response against an antigen. WO2008/017517 describes a new strategy using peptides comprising an MHC class II antigen of a given antigenic protein and an oxidoreductase motif. These peptides convert CD4+ T cells into a cell type with cytolytic properties called cytolytic CD4+ T cells. These cells are capable to kill via triggering apoptosis those antigen presenting cells (APC), which present the antigen from which the peptide is derived. WO2008/017517 demonstrates this concept for allergies and auto-immune diseases such as type I diabetes. Herein insulin can act as an auto-antigen.

WO2009101207 and Carlier et al. (2012) Plus one 7,10 e45366 further describe the antigen specific cytolytic CD4+ T cells in more detail.

WO2009101206 describes the use of peptides with an oxidoreductase motif and an MCH class II epitope of a soluble allo-antigen to prevent an immune response against such antigen when used in replacement therapies (e.g. unwanted immune response against injected insulin in diabetes patents).

WO2016059236 discloses further modified peptides wherein an additional Histidine is present in the proximity of the oxidoreductase motif.

In addition to the peptides comprising an MHC class II epitope of an allergen or antigen, WO2012069568A2 further disclosed the possibility of using NKT cell epitopes, binding the CD1d receptor and resulting in activation of cytolytic antigen-specific NKT cells, which have been shown to eliminate, in an antigen-specific manner, APC presenting said specific antigen.

Both strategies are building upon the use of oxidoreductase motifs of the [CST]-XX-C or C-XX-[CST] type. In order to improve the efficacy of a treatment using such immunogenic peptides, the search for more active peptides continues.

### SUMMARY OF THE INVENTION

The present invention provides novel immunogenic peptides comprising a T-cell epitope of an antigen and an oxidoreductase motif. After conducting extensive experiments, the inventors have identified a new type of oxidoreductase motifs with improved therapeutic activities in terms of cytolytic CD4+ T cells induction and in-vivo therapeutic or prophylactic effect when compared to the traditionally used HC-XX-[CST] (SEQ ID NO: 118) or H[CST]-XX-C(SEQ ID NO: 119) oxidoreductase motifs. When combining these with an (additional) basic (charged) amino acid residue before, inside or after said new motifs, the activity can be further improved. These effects are displayed in the Figures and explained in the Examples section. Also the effect at the cellular level and in mouse model systems has been tested and confirms the improved activity of the immunogenic peptides of the invention.

The present invention relates to the following aspects:

Aspect 1: An immunogenic peptide, said immunogenic peptide comprising:

a) an oxidoreductase motif;
b) a T-cell epitope of an antigenic protein; and
c) a linker between a) and b) of between 0 and 7 amino acids, preferably of between 0 and 4 amino acids;
wherein said oxidoreductase motif a) has the following general structure:

Zₘ-[CST]-Xₙ-C- (SEQ ID NO: 56 to 80), or Zₘ-C-Xₙ-[CST]- (SEQ ID NO: 81 to 105),

wherein X or Z is any amino acid, preferably a basic amino acid, more preferably selected from the group comprising: K, H, R and a non-natural basic amino acid, preferably K or H, more preferably K; wherein n is an integer selected from the group comprising: 1, 0, 3, 4, 5 or 6, preferably 1, 0, or 3, wherein m is an integer between 0 and 3, preferably 0 or 1;
wherein C, S and T respectively indicate a cysteine, serine and threonine amino acid residue and wherein [CST] indicates that any one of C, S, or T can be selected, preferably a non-modified natural amino acid as defined herein;
wherein said antigenic protein is an auto-antigen, a soluble allofactor, an alloantigen shed by the graft, an antigen of an intracellular pathogen, an antigen of a viral vector used for gene therapy or gene vaccination, a tumor-associated antigen or an allergen.

The immunogenic peptides as defined herein have oxidoreductase activity.

In any one of the general structures, formula's or sequences defining the oxidoreductase motif, the hyphen (-) at the end indicates the point of attachment of the oxidoreductase motif to the N-terminal end of the linker or the epitope, or to the C-terminal end of the linker or the T cell epitope.

More preferably, said antigenic protein is an autoantigen involved in type-1 diabetes (T1D), demyelinating disorders such as multiple sclerosis (MS) or neuromyelitis optica (NMO), or in rheumatoid arthritis (RA).

Non-Limiting Examples of Autoantigens Involved in T1D.
Source: Mallone R et al., Clin Dev Immunol. 2011:513210.

| | UniProtKB | Examples of |
|---|---|---|
| Auto-antigen | identifier | T-cell epitopes |
| Insulin | P01308 | LALEGSLQK |
| | | (SEQ ID NO: 3) |
| GAD65 | Q9UGI5 | |
| GAD67 | Q99259 | |
| IA-2 (ICA512) | Q16849 | |
| IA-2 (beta/phogrin) | Q92932 | |
| IGRP | Q9NQR9 | |
| Chromogranin | P10645 | |
| ZnT8 | Q8IWU4 | |
| HSP-60 | P10809 | |

Non-Limiting Examples of Autoantigens Involved in MS.

| | UniProtKB | | Examples of |
|---|---|---|---|
| Auto-antigen | identifier | Comments | T-cell epitopes |
| Myelin oligodendrocyte | Q16653 | Immunodominant | FLRVPCWKI |
| glycoprotein (MOG) | | epitopes in the following | (SEQ ID NO: 4) |
| | | regions: amino acids | or FLRVPSWKI |
| | | 1-22, 35-55 and 92-106. | (SEQ ID NO: 5) |
| Myelin basic protein | P02686 | Immunodominant | |
| (MBP) | | epitopes in the amino | |
| | | acids 85-99 region. | |
| Proteolipid protein | P60201 | Immunodominant | |
| (PLP) | | epitopes in the following | |
| | | regions: amino acids | |
| | | 31-70, 91-120 and 178-228. | |
| Myelin-oligodendrocytic | Q13875 | Immunodominant | |
| basic protein (MOBP) | | epitopes in the amino | |
| | | acids 15-36 region. | |
| Oligodendrocyte-specific | O75508 | Immunodominant | |
| protein (OSP) | | epitopes in the amino | |
| | | acids 179-207 region. | |

Examples of Autoantigens Involved in RA.

| Auto-antigen | UniProtKB identifier |
|---|---|
| GRP78 | P11021 |
| HSP60 | P10809 |
| 60 kDa chaperonin 2 | P9WPE7 |
| Gelsolin | P06396 |
| Chitinase-3-like protein 1 | P36222 |
| Cathepsin S | P25774 |
| Serum albumin | P02768 |
| Cathepsin D | P07339 |

Examples of Autoantigens Involved in NMO

| | | | Examples |
|---|---|---|---|
| | | | of T- |
| Auto- | UniProtKB | | cell |
| antigen | identifier | Comments | epitopes |
| Myelin | Q16653 | Immuno- | FLRVPCWKI |
| oligodendrocyte | | dominant | (SEQ |
| glycoprotein | | epitopes | ID NO: 4) |
| (MOG) | | in the | or |
| | | following | FLRVPSWKI |
| | | regions: | (SEQ |
| | | amino acids | ID NO: 5) |
| | | 1-22, 35-55 | |
| | | and 92-106. | |

Aspect 2: The immunogenic peptide according to aspect 1, wherein said oxidoreductase motif is not part of a repeat of the standard C-XX-[CST] or [CST]-XX-C oxidoreductase motifs such as repeats of said motif which can be spaced from each other by one or more amino acids (e.g. CXXC X CXXC X CXXC (SEQ ID NO: 6)), as repeats which are adjacent to each other (CXXCCXXCCXXC (SEQ ID NO: 7)) or as repeats which overlap with each other CXXCXXCXXC (SEQ ID NO: 8) or CXCCXCCXCC (SEQ ID NO: 9)), especially when n is 0 or 1 and m is 0.

Aspect 3: The immunogenic peptide according to aspects 1 or 2, wherein at least one X in the motif is a basic amino acid preferably selected from the group comprising: K, H, R or a non-natural basic amino acid, preferably K or R, more preferably R.

More particularly:

when n is 1, then X is preferably a basic amino acid preferably selected from the group comprising: K, H, R or a non-natural basic amino acid, preferably K or R, more preferably R;
when n is 3 or more, then at least one X is preferably a basic amino acid preferably selected from the group comprising: K, H, R or a non-natural basic amino acid, preferably K or R, more preferably R.

Aspect 4: The immunogenic peptide according to anyone of aspects 1 to 3, wherein at least one Z in the motif is a basic amino acid preferably selected from the group comprising: K, H, R or a non-natural basic amino acid, preferably K or H.

More particularly:

when m is 1, then Z is preferably a basic amino preferably selected from the group comprising: K, H, R or a non-natural basic amino acid, preferably K or H; when m is 2 or 3, then at least one Z is preferably a basic amino acid preferably selected from the group comprising: K, H, R or a non-natural basic amino acid, preferably K or H.

In some embodiments where m is 2 or 3, at least two Z's are preferably a basic amino acid preferably selected from the group comprising: K, H, R or a non-natural basic amino acid, preferably K or H.

Aspect 5: The immunogenic peptide according to anyone of aspects 1 to 4, wherein:

n is 0 and wherein m is 0, 1, 2 or 3; or
wherein n is 1 and m is 0 or 1;
wherein X is a basic amino acid, more preferably selected from the group comprising: K, H, R and a non-natural basic amino acid, preferably K or R, more preferably R;
wherein Z, when present, is a basic amino acid, more preferably selected from the group comprising: K, H, R and a non-natural basic amino acid, preferably K or H, more preferably K; or
wherein n is 3 and m is 0 or 1;
wherein at least one X is a basic amino acid, more preferably selected from the group comprising: K, H, R and a non-natural basic amino acid, preferably K or R, more preferably R; wherein Z, when present, is a basic amino acid, more preferably selected from the group comprising: K, H, R and a non-natural basic amino acid, preferably K or H, more preferably K.

Aspect 6: The immunogenic peptide according to aspect 1, wherein said antigenic protein is (pro)insulin, GAD65, GAD67, IA-2 (ICA512), IA-2 (beta/phogrin), IGRP, Chromogranin, ZnT8, or HSP-60 for use in treating and/or prevention of type-1 diabetes (T1D); or

wherein said antigenic protein is Myelin oligodendrocyte glycoprotein (MOG), Myelin basic protein (MBP), Proteolipid protein (PLP), Myelin-oligodendrocytic basic protein (MOBP), or Oligodendrocyte-specific protein (OSP), for use in treating and/or prevention of a demyelinating disorder such as multiple sclerosis (MS); or
wherein said antigenic protein is Myelin oligodendrocyte glycoprotein (MOG) for use in treating and/or prevention of a demyelinating disorder such as neuromyelitis optica (NMO); or
wherein said antigenic protein is GRP78, HSP60, 60 kDa chaperonin 2, Gelsolin, Chitinase-3-like protein 1, or Cathepsin S, for use in treating and/or prevention of rheumatoid arthritis (RA).

Aspect 7. The immunogenic peptide according to any one of aspects 1 to 6, wherein said T cell epitope of an antigenic protein is an NKT cell epitope or an MHC class II T cell epitope.

Aspect 8. The immunogenic peptide according to any one of aspects 1 to 7, wherein said T cell epitope of an antigenic protein is an NKT cell epitope having a length of between 7 and 25 amino acids; or wherein said T cell epitope of an antigenic protein is an MHC class II T cell epitope having a length of between 7 and 25 amino acids, preferably between 9 and 25 amino acids.

Aspect 9: The immunogenic peptide according to any one of aspects 1 to 8, wherein said antigenic protein comprising an NKT cell epitope has a length of between 7 and 50 amino acids, and/or wherein said immunogenic peptide comprising an MHC class II T cell epitope has a length of between 7 and 50 amino acids, preferably between 9 and 50 amino acids.

Aspect 10: The immunogenic peptide according to any one of aspects 1 to 9, wherein the linker is of between 0 and 4 amino acids.

Aspect 11: The immunogenic peptide according to any one of aspects 1 to 10, wherein the oxidoreductase motif is located N-terminally from the epitope or C-terminally from the epitope, and/or wherein the oxidoreductase motif is located at the N-terminal or C-terminal end of the immunogenic peptide.

Aspect 12: The immunogenic peptide according to any one of aspects 1 to 11, wherein said oxidoreductase motif does not naturally occur within a region of 11 amino acids N-terminally or C-terminally of the T-cell epitope in said antigenic protein.

Aspect 13: The immunogenic peptide according to any one of aspects 1 to 12, wherein the T-cell epitope does not naturally comprise said oxidoreductase motif.

Aspect 14: The immunogenic peptide according to any one of aspects 1 to 13, wherein in said oxidoreductase motif, n is 0 and wherein m is 0, 1, 2 or 3; preferably 0 or 1;

wherein Z, when present, is a basic amino acid, more preferably selected from the group comprising: K, H, R and a non-natural basic amino acid such as but not limited to L-ornithine, preferably K or H, more preferably K.

Aspect 15: The immunogenic peptide according to any one of aspects 1 to 13, wherein in said oxidoreductase motif, n is 1 and wherein m is 0, 1, 2 or 3; preferably 0 or 1;

wherein X is a basic amino acid, more preferably selected from the group comprising: K, H, R and a non-natural basic amino acid such as but not limited to L-omithine, preferably K or R, more preferably R;
wherein Z, when present, is a basic amino acid, more preferably selected from the group comprising: K, H, R and a non-natural basic amino acid such as but not limited to L-ornithine, preferably K or H, more preferably K. Preferred embodiments are those wherein the oxidoreductase motif comprises or consists of KCRC, HCRC, or RCRC.

Aspect 16: The immunogenic peptide according to any one of aspects 1 to 13, wherein in said oxidoreductase motif, n is 3 and wherein m is 0, 1, 2 or 3; preferably 0 or 1;

wherein at least one X is a basic amino acid, more preferably selected from the group comprising: K, H, R and a non-natural basic amino acid such as but not limited to L-ornithine, preferably K or R, more preferably R;
wherein Z, when present, is a basic amino acid, more preferably selected from the group comprising: K, H, R and a non-natural basic amino acid such as but not limited to L-ornithine, preferably K or H, more preferably K.

Aspect 17: The immunogenic peptide according to aspect 16, wherein at least one X in the motif is P or Y, preferably wherein said motif comprises RPY, KPY or HPY.

Aspect 18: The immunogenic peptide according to any one of aspects 1 to 13, wherein in said oxidoreductase motif, n is 0 or 1; m is 1;

wherein Z is a basic amino acid selected from the group consisting of: K, H, and R, preferably K or H, more preferably K; and
wherein X, when present, is a basic amino acid selected from the group consisting of: K, H, and R, preferably K or R, more preferably R.

Aspect 19: The immunogenic peptide according to any one of aspects 1 to 13, wherein in said oxidoreductase motif, n is 3; m is 1;

wherein Z is a basic amino acid selected from the group consisting of: K, H, and R, preferably K or H, more preferably K; and
wherein at least one X is a basic amino acid selected from the group consisting of: K, H, and R, preferably K or R, more preferably R.

Aspect 20. The immunogenic peptide according to any one of aspects 1 to 13, wherein said immunogenic peptide comprises a T-cell epitope derived from the Myelin-oligodendrocyte glycoprotein (MOG) antigen amino acid sequence selected from YRSPFSRW (SEQ ID NO: 36) and YRPPFSRW (human SEQ ID NO: 123), and comprises as a linker the amino acid sequence GW and comprises as a flanker the amino acid sequence HLYR (SEQ ID NO: 122),

Aspect 21. The immunogenic peptide according to any one of aspects 1 to 13, wherein said immunogenic peptide comprises a T-cell epitope derived from the Myelin-oligodendrocyte glycoprotein (MOG) antigen amino acid sequence selected from FLRVPCWKI (SEQ ID NO: 124), and FLRVPSWKI (SEQ ID NO: 125), and comprises as a linker the amino acid sequence VRY and comprises as a flanker an amino acid sequence selected from: TLF, TLFK (SEQ ID NO: 126), or TLFKK (SEQ ID NO: 127).

Aspect 22. The immunogenic peptide according to any one of aspects 1 to 13, wherein said immunogenic peptide is selected from the group consisting of:

(SEQ ID NO: 128)
KHCPYCVRYFLRVPSWKITLFKK,
(SEQ ID NO: 129)
KHCPYCVRYFLRVPCWKITLFKK.
(SEQ ID NO: 130)
HCPYCVRYFLRVPSWKITLF,
(SEQ ID NO: 131)
HCPYCVRYFLRVPCWKITLF,
(SEQ ID NO: 38)
HCPYCGWYRSPFSRVVHLYR,
(SEQ ID NO: 43)
KCRCGWYRSPFSRWHLYR,
and
(SEQ ID NO: 47)
KCRPYCGWYRSPFSRWHLYR.

Aspect 23, A polynucleotide (nucleic acid molecule) encoding the immunogenic peptide according to any one of aspects 1 to 22, preferably selected from isolated desoxyribonucleic acid (DNA), plasmid DNA (pDNA), coding DNA (cDNA), ribonucleic acid (RNA), messenger RNA (mRNA) or modified versions thereof. In some embodiments, said nucleic acid can be part of an expression cassette, optionally incorporated in a (viral) vector or plasmid that can be used for gene-therapy or can be present in the form of encapsulated or naked DNA or RNA to be administered according to techniques known in the pharmaceutical and gene therapeutic field.

Aspect 24. The immunogenic peptide according to any one of aspects 1 to 22, or the polynucleotide according to aspect 23, for use in treating and/or prevention of an autoimmune disease, an infection with an intracellular pathogen, a tumor, an allograft rejection, or an immune response to a soluble allofactors, to an allergen exposure or to a viral vector used for gene therapy or gene vaccination.

Preferably, the immunogenic peptide according to any one of aspects 1 to 22 or the polynucleotide according to aspect 23 is used for treating of, ameliorating the symptoms of, and/or preventing of an autoimmune disease, preferably type-1 diabetes (T1D), a demyelinating disorder such as multiple sclerosis (MS) or neuromyelitis optica (NMO), or rheumatoid arthritis (RA).

Aspect 25: A method for preparing an immunogenic peptide according to any one of aspects 1 to 22, comprising the steps of:

(a) providing a peptide sequence consisting of a T-cell epitope of said antigenic protein, and
(b) linking to said peptide sequence said oxidoreductase motif, such that said motif and said epitope are either adjacent to each other or separated by a linker of between 0 and 7 amino adds, and such that Z corresponds to the N- or C-terminal end of the immunogenic peptide.

Aspect 26: A method for obtaining a population of antigen-specific cytolytic CD4+ T cells, against APC presenting said antigen, the method comprising the steps of:
providing peripheral blood cells,
contacting said cells with an immunogenic peptide according to any one of aspects 1 to 22 or with the polynucleotide of aspect 23; and
expanding said cells in the presence of IL-2.

Aspect 27: A method for obtaining a population of antigen-specific NKT cells, the method comprising the steps of:
providing peripheral blood cells,
contacting said cells with an immunogenic peptide according to any one of aspects 1 to 22, or with the polynucleotide of aspect 23; and
expanding said cells in the presence of IL-2.

Aspect 28: A method for obtaining a population of antigen-specific cytolytic CD4+ T cells, against APC presenting said antigen, the method comprising the steps of:
providing an immunogenic peptide according to any one of aspects 1 to 22, or with the polynucleotide of aspect 23,
administering said peptide to a subject, and
obtaining said population of antigen-specific cytolytic CD4+ T cells from said subject.

Aspect 29: A method for obtaining a population of antigen-specific NKT cells, the method comprising the steps of:
providing an immunogenic peptide according to any of aspects 1 to 22, or with the polynucleotide of aspect 23;
administering said peptide to a subject, and
obtaining said population of antigen-specific NKT cells from said subject.

Aspect 30: The population of antigen-specific cytolytic CD4+ T cells or NKT cells obtainable by the method of any one of aspects 25 to 29 for use in medicine, more particularly for use in the treatment and/or prevention of type-1 diabetes (T1D), a demyelinating disorder such as multiple sclerosis (MS) or neuromyelitis optica (NMO), or rheumatoid arthritis (RA).

Aspect 31: A method of treating of, ameliorating the symptoms of, and/or preventing of an autoimmune disease in a subject, preferably type-1 diabetes (T1D), a demyelinating disorder such as multiple sclerosis (MS) or neuromyelitis optica (NMO), or rheumatoid arthritis (RA), comprising the steps of administering the immunogenic peptide according to anyone of aspects 1 to 22, the polynucleotide of aspect 23, or the cell population according to aspect 30 to said subject.

Aspect 32: A method of treating of, ameliorating the symptoms of and/or preventing of an autoimmune disease in a subject, preferably type-1 diabetes (T1D); a demyelinating disorder such as multiple sclerosis (MS) or neuromyelitis optica (NMO); or rheumatoid arthritis (RA), comprising the steps of:
providing peripheral blood cells of said subject,
contacting said cells with an antigenic peptide according to any one of aspects 1 to 22, or with the polynucleotide of aspect 23,
expanding said cells in vitro, and
administering said expanded cells to said individual.

Aspect 33: Particularly preferred examples of oxidoreductase motifs to be read into any one of aspects 1 to 22 or any one of the embodiments disclosed herein are:

### CC-;

[HKR]CC-, such as HCC-, RCC-, KCC-;
C[KRH]C-, such as CRC-, CKC-, CHC-;
[KHR]C[KRH]C-, such as KCRC-, KCKC-, KCHC-, RCRC-, RCKC-, RCHC-, HCRC-, HCKC-, HCHC-, corresponding to SEQ ID NO: 10 to 19;
C[KHR]XXC-, such as CRXXC-, CHXXC-, CKXXC-, corresponding to SEQ ID NO: 20 to 23, preferably wherein one X in the motif is P or Y;
-[KHR]C[KHR]XXC-, KCRXXC-, KCHXXC-, KCKXXC-, HCRXXC-, HCHXXC-, HCKXXC-, RCRXXC-, RCHXXC-, RCKXXC-, corresponding to SEQ ID NO: 24 to 33, preferably wherein one X in the motif is P or Y;
and the like.

Particularly preferred oxidoreductase motifs are KCRC- (SEQ ID NO: 11) and KCRPYC-(SEQ ID NO: 54), KCC-, CRC-, and CRPYC- (SEQ ID NO: 55). In a preferred embodiment of any one of aspects 1 to 30, the linker comprises at least 1 amino acid, at least 2 amino acids, at least 3 amino acids, or at least 4 amino acids. Preferably, said linker comprises between 1 and 7 amino acids, such as between 2 and 7 amino acids, between 3 and 7 amino acids, or between 4 and 7 amino acids.

In another preferred embodiment of any one of aspects 1 to 30, the T-cell epitope does not comprise a basic amino acid at its N-terminal end, i.e. immediately adjacent to the linker or oxidoreductase motif, more particularly in case the linker is absent or only comprises 1 or 2 amino acids. More preferably, in all aspects, the T-cell epitope does not comprise a basic amino acid at its N-terminal end, i.e. immediately adjacent to the linker or oxidoreductase motif, more particularly in case the linker is absent or only comprises 1 or 2 amino acids.

In a further embodiment of any one of aspects 1 to 30, the T-cell epitope does not comprise a basic amino acid in position 1, 2 and/or 3 counted from its N-terminal end, i.e. immediately adjacent to the linker or oxidoreductase motif, more particularly in case the linker is absent or only comprises 1 or 2 amino acids.

In any one of aspects 1 to 30, the oxidoreductase motif forms the N-terminal end of the immunogenic peptide. In an alternative set of embodiments, the oxidoreductase motif forms the C-terminal end of the immunogenic peptide.

The peptides of the present invention have a greater capacity to generate cytolytic CD4+ T cells compared to the prior art peptides, and have a greater capacity to treat or prevent diseases than prior art peptides.

Patients being treated for MS typically have HLA HLA-DRB1* types selected from the group consisting of: HLA-DRB1*15:01, HLA-DRB1*03:01, HLA-DRB1*04:01, and HLA-DRB1*07:01, preferably HLA-DRB1* 15:01.

Patients being treated for NMO typically have HLA types selected from the group consisting of: HLA-DRB1*03:01 and HLA-DPB1*05:01 (for Asia).

Patients being treated for T1D, typically have HLA types selected from the group consisting of: HLA-DRB1*03:01 and 04:01.

Patients being treated for RA typically have HLA types selected from the group consisting of: HLA-DRB1*01:01, 04:01, and 04:04.

### BRIEF DESCRIPTION OF THE FIGURES

FIG. 1: represents the kinetics of the redox activities of the immunogenic peptides. DTT is used as a positive control, while Blank represents the assay buffer. The results are expressed in Relative Fluorescent Units (RFU). Results from one duplicate are shown. See text for details.

FIG. 2: A) represents EAE Mean clinical score (+SEM) from Day 7 to Day 28 in mice prophylactically treated with alum or IMCY-0189 or IMCY-0453. B) represents AUC of the EAE clinical scores from Day 7 to Day 28. C) represents MMS (+SD) of EAE clinical score from Day 7 to Day 28. See text for details.

FIG. 3: represents AUC of the EAE clinical scores from Day 7 to Day 28 in mice treated with different peptides or with Alum alone.

FIG. 4: represents MMS (+SD) of EAE clinical score from Day 7 to Day 28 in mice treated with different peptides or with Alum alone.

FIG. 5: represents blinded evaluation of clinical EAE scoring (0-5) performed daily from day 7 to day 28. Mice were injected with MOG₃₅₋₅₅ to induce EAE at day 0, and were left untreated or therapeutically treated with IMCY-0189, IMCY-0453 or IMCY-0455 (see table 6 for details). The mean clinical score was determined each day for each group of mice.

FIG. 6: represents AUC calculated from EAE scores displayed in FIG. 5 for each group of mice. Significant differences are referred as follows: *p<0.05, **p<0.01, ***p<0.001, ****p<0.0001.

FIG. 7: represents MMS calculated from EAE scores displayed in FIG. 5 for each group of mice. Significant differences are referred as follows: *p<0.05, **p<0.01, ***p<0.001, ****p<0.0001.

FIG. 8: represents inflammation levels for each group of mice presented in table 6. Inflammatory foci of approximately 20 cells were counted in each H&E stained section. When inflammatory infiltrates consisted of more than 20 cells, an estimate was made of how many foci of 20 cells were present. Significant differences are referred as follows: *p<0.05, **p<0.01, ***p<0.001, ****p<0.0001.

FIG. 9: represents demyelination levels for each group of mice presented in table 6. Demyelination was scored in each anti-MBP (using immunohistochemistry) stained section. The demyelination score represents an estimate of demyelinated area for each section. Significant differences are referred as follows: *p<0.05, **p<0.01, ***p<0.001, ****p<0.0001.

FIG. 10: represents serum neurofilaments levels for each group of mice presented in table 6. Neurofilament light (NF-L) protein levels were quantified at Quanterix™ through the NF-light Simoa® assay advantage kit. Significant differences are referred as follows: *p<0.05, **p<0.01, ***p<0.001, ****p<0.0001.

FIG. 11: represents the percentage of Ag-specific CD4 T cells after 16 h co-culture with APC in the absence and presence of experimental and control peptides. 2D2-Total CD4+ cells were co-cultured with C57BL6 APC (Ratio 1:1, biological duplicates) in the presence of each of the variant of IMCY-0189 (IMCY-0451 to IMCY-0456) for 16 h. Control conditions were the addition of IMCY-0017 (wt MOG35-55), IMCY-0069 (modified murine pre-proinsulin), IMCY-0257 (modified DBY antigen) and no peptide addition. After the incubation time, the percentage of CD4+CD154+ cells were determined by flow cytometry. The dotted line shows the differences between the percentage of Ag-specific CD4+ T cells for reference conventional Imotope (IMCY-0189, black column) compared with all experimental and control conditions (white columns). SD for the FACS-measurement of biological duplicates was shown for every condition.

FIG. 12: represents sIL2 by 2D2-Total CD4 T cells after 24 h co-culture with APC in the absence and presence of experimental and control peptides. 2D2-Total CD4+ cells were co-cultured with C57BL6 APC (Ratio 1:1, biological duplicates) in the presence of each variant of IMCY-0189 (IMCY-0451 to IMCY-0456) and control peptides, separately, for 24 h. After the incubation time, SN was collected and the amount of IL2 was measured by flow cytometry using Legend Plex kit. Control conditions were the addition of IMCY-0017 (wt MOG35-55), IMCY-0069 (modified murine pre-proinsulin), IMCY-0257 (modified DBY antigen) and no peptide addition. The dotted line shows the differences between the amount of sIL2 (pg/ml) for reference conventional Imotope (IMCY-0189, black column) versus all experimental and control conditions (White columns). SD for the FACS-measurement of biological duplicates is shown for every condition.

FIG. 13: represents the percentage of Ag-specific CD4 T cells expressing Granzyme A, Granzyme B, CD107a/b alone or in combination, 16 h post stimulation with APC in the absence and presence of experimental peptides.

At S2, 2D2-Total CD4+ cells were co-cultured with C57BL6 APC (Ratio 1:1, biological duplicates) in absence and presence of each variant of IMCY-0189 (IMCY-0451 to IMCY-0456) for 16 h. After the incubation time percentage of CD4+ cells expressing lytic markers were determined by flow cytometry for each cell line. The measurements for the IMCY-0017 (wt) was removed from every other measurement for modified peptides to pronounce the effect of oxidoreductase motif on the induction of lytic markers. The dotted line shows the differences between the percentage of lytic markers on CD4+ T cells for reference conventional Imotope (IMCY-0189) compared with all experimental modified peptides.

### DETAILED DESCRIPTION OF THE INVENTION

The present invention will be described with respect to particular embodiments, although the invention is not limited thereto but only by the claims. Any reference signs in the claims shall not be construed as limiting the scope. The following terms or definitions are provided solely to aid in the understanding of the invention. Unless specifically defined herein, all terms used herein have the same meaning as they would have to one skilled in the art of the present invention. The definitions provided herein should not be construed to have a scope less than the one understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise. The term “any” when used in relation to aspects, claims or embodiments as used herein refers to any single one (i.e. anyone) as well as to all combinations of said aspects, claims or embodiments referred to.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. Said terms also encompass the embodiments “consisting essentially of” and “consisting of”.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

As used herein, the term “for use” as used in “composition for use in treatment of a disease” shall disclose also the corresponding method of treatment and the corresponding use of a preparation for the manufacture of a medicament for the treatment of a disease”.

The term “peptide” as used herein refers to a molecule comprising an amino acid sequence of between 9 and 200 amino acids, connected by peptide bonds, but which can comprise non-amino acid structures.

The term “Immunogenic peptide” as used herein refers to a peptide that is immunogenic, i.e. that comprises a T-cell epitope capable of eliciting an immune response.

Peptides according to the invention can contain any of the conventional 20 amino acids or modified versions thereof, or can contain non-naturally occurring amino-acids incorporated by chemical peptide synthesis or by chemical or enzymatic modification.

The term “antigen” as used herein refers to a structure of a macromolecule, typically a protein (with or without polysaccharides) or made of proteic composition comprising one or more hapten(s) and comprising T or NKT cell epitopes.

The term “antigenic protein” as used herein refers to a protein comprising one or more T or NKT cell epitopes. An auto-antigen or auto-antigenic protein as used herein refers to a human or animal protein or fragment thereof present in the body, which elicits an immune response within the same human or animal body.

The term “food or pharmaceutical antigenic protein” refers to an antigenic protein present in a food or pharmaceutical product, such as in a vaccine.

The term “epitope” refers to one or several portions (which may define a conformational epitope) of an antigenic protein which is/are specifically recognised and bound by an antibody or a portion thereof (Fab′, Fab₂′, etc.) or a receptor presented at the cell surface of a B-, or T-, or NKT cell, and which is able, by said binding, to induce an immune response.

The term “T cell epitope” in the context of the present invention refers to a dominant, sub-dominant or minor T cell epitope, i.e. a part of an antigenic protein that is specifically recognised and bound by a receptor at the cell surface of a T lymphocyte. Whether an epitope is dominant, sub-dominant or minor depends on the immune reaction elicited against the epitope. Dominance depends on the frequency at which such epitopes are recognised by T cells and able to activate them, among all the possible T cell epitopes of a protein. The T cell epitope can be an epitope recognised by MHC class II molecules or an NKT cell epitope recognised by a CD1d molecule.

A T cell epitope can be an epitope recognised by MHC class II molecules, typically consists of a sequence of 9 amino acids that fits in the groove of the MHC II molecule. Within a peptide sequence representing an MHC class II T cell epitope, the amino acids in the epitope can be numbered P1 to P9, amino acids N-terminal of the epitope are numbered P-1, P-2 and so on, amino acids C terminal of the epitope are numbered P+1, P+2 and so on. Peptides recognised by MHC class II molecules and not by MHC class I molecules are referred to as MHC class II restricted T cell epitopes.

The identification and selection of a T-cell epitope from antigenic proteins is known to a person skilled in the art.

To identify an epitope suitable in the context of the present invention, isolated peptide sequences of an antigenic protein are tested by, for example, T cell biology techniques, to determine whether the peptide sequences elicit a T cell response. Those peptide sequences found to elicit a T cell response are defined as having T cell stimulating activity.

Human T cell stimulating activity can further be tested by culturing T cells obtained from e.g. an individual having T1D, with a peptide/epitope derived from the auto-antigen involved in T1D and determining whether proliferation of T cells occurs in response to the peptide/epitope as measured, e.g., by cellular uptake of tritiated thymidine. Stimulation indices for responses by T cells to peptides/epitopes can be calculated as the maximum CPM in response to a peptide/epitope divided by the control CPM. A T cell stimulation index (S.I.) equal to or greater than two times the background level is considered “positive.” Positive results are used to calculate the mean stimulation index for each peptide/epitope for the group of peptides/epitopes tested.

Non-natural (or modified) T-cell epitopes can further optionally be tested on their binding affinity to MHC class 11 molecules. This can be performed in different ways. For instance, soluble HLA class II molecules are obtained by lysis of cells homozygous for a given class II molecule. The latter is purified by affinity chromatography. Soluble class II molecules are incubated with a biotin-labelled reference peptide produced according to its strong binding affinity for that class II molecule. Peptides to be assessed for class II binding are then incubated at different concentrations and their capacity to displace the reference peptide from its class II binding is calculated by addition of neutravidin.

In order to determine optimal T cell epitopes by, for example, fine mapping techniques, a peptide having T cell stimulating activity and thus comprising at least one T cell epitope as determined by T cell biology techniques is modified by addition or deletion of amino acid residues at either the amino- or carboxy-terminus of the peptide and tested to determine a change in T cell reactivity to the modified peptide. If two or more peptides which share an area of overlap in the native protein sequence are found to have human T cell stimulating activity, as determined by T cell biology techniques, additional peptides can be produced comprising all or a portion of such peptides and these additional peptides can be tested by a similar procedure. Following this technique, peptides are selected and produced recombinantly or synthetically. T cell epitopes or peptides are selected based on various factors, including the strength of the T cell response to the peptide/epitope (e.g., stimulation index) and the frequency of the T cell response to the peptide in a population of individuals.

Additionally and/or alternatively, one or more in vitro algorithms can be used to identify a T cell epitope sequence within an antigenic protein. Suitable algorithms include, but are not limited to those described in Zhang et al. (2005) Nucleic Acids Res 33, W180-W183 (PREDBALB); Salomon & Flower (2006) BMC Bioinformatics 7, 501 (MHCBN); Schuler et al. (2007) Methods Mol. Biol. 409, 75-93 (SYFPEITHI); Donnes & Kohlbacher (2006) Nucleic Acids Res. 34, W194-W197 (SVMHC); Kolaskar & Tongaonkar (1990) FEBS Lett. 276, 172-174, Guan et al. (2003) Appl. Bioinformatics 2, 63-66 (MHCPred) and Singh and Raghava (2001) Bioinformatics 17, 1236-1237 (Propred). More particularly, such algorithms allow the prediction within an antigenic protein of one or more octa- or nonapeptide sequences which will fit into the groove of an MHC II molecule and this for different HLA types.

The term “MHC” refers to “major histocompatibility antigen”. In humans, the MHC genes are known as HLA (“human leukocyte antigen”) genes. Although there is no consistently followed convention, some literature uses HLA to refer to HLA protein molecules, and MHC to refer to the genes encoding the HLA proteins. As such the terms “MHC” and “HLA” are equivalents when used herein. The HLA system in man has its equivalent in the mouse, i.e., the H2 system. The most intensely-studied HLA genes are the nine so-called classical MHC genes: HLA-A, HLA-B, HLA-C, HLA-DPA1, HLA-DPB1, HLA-DQA1, HLAs DQB1, HLA-DRA, and HLA-DRB1. In humans, the MHC is divided into three regions: Class I, II, and III. The A, B, and C genes belong to MHC class I, whereas the six D genes belong to class II. MHC Cass I molecules are made of a single polymorphic chain containing 3 domains (alpha 1, 2 and 3), which associates with beta 2 microglobulin at cell surface. Class II molecules are made of 2 polymorphic chains, each containing 2 chains (alpha 1 and 2, and beta 1 and 2).

Patients being treated for MS typically have HLA HLA-DRB1* types selected from the group consisting of: HLA-DRB1*15:01, HLA-DRB1*03:01, HLA-DRB1*04:01, and HLA-DRB1*07:01, preferably HLA-DRB1* 15:01.

Patients being treated for NMO typically have HLA types selected from the group consisting of: HLA-DRB1*03:01 and HLA-DPB1*05:01 (for Asia).

Patients being treated for T1D, typically have HLA types selected from the group consisting of: HLA-DRB1*03:01 and 04:01.

Patients being treated for RA typically have HLA types selected from the group consisting of: HLA-DRB1*01:01, 04:01, and 04:04.

Class I MHC molecules are expressed on virtually all nucleated cells.

Peptide fragments presented in the context of class I MHC molecules are recognised by CD8+ T lymphocytes (cytolytic T lymphocytes or CTLs). CD8+ T lymphocytes frequently mature into cytolytic effectors which can lyse cells bearing the stimulating antigen. Class II MHC molecules are expressed primarily on activated lymphocytes and antigen-presenting cells. CD4+ T lymphocytes (helper T lymphocytes or Th) are activated with recognition of a unique peptide fragment presented by a class II MHC molecule, usually found on an antigen-presenting cell like a macrophage or dendritic cell. CD4+ T lymphocytes proliferate and secrete cytokines such as IL-2, IFN-gamma and IL-4 that support antibody-mediated and cell mediated responses.

Functional HLAs are characterised by a deep binding groove to which endogenous as well as foreign, potentially antigenic peptides bind. The groove is further characterised by a well-defined shape and physico-chemical properties. HLA class I binding sites are closed, in that the peptide termini are pinned down into the ends of the groove. They are also involved in a network of hydrogen bonds with conserved HLA residues. In view of these restraints, the length of bound peptides is limited to 8, 9 or 10 residues. However, it has been demonstrated that peptides of up to 12 amino acid residues are also capable of binding HLA class I. Comparison of the structures of different HLA complexes confirmed a general mode of binding wherein peptides adopt a relatively linear, extended conformation, or can involve central residues to bulge out of the groove.

In contrast to HLA class I binding sites, class II sites are open at both ends. This allows peptides to extend from the actual region of binding, thereby “hanging out” at both ends. Class II HLAs can therefore bind peptide ligands of variable length, ranging from 9 to more than 25 amino acid residues. Similar to HLA class I, the affinity of a class II ligand is determined by a “constant” and a “variable” component. The constant part again results from a network of hydrogen bonds formed between conserved residues in the HLA class II groove and the main-chain of a bound peptide. However, this hydrogen bond pattern is not confined to the N-and C-terminal residues of the peptide but distributed over the whole chain. The latter is important because it restricts the conformation of complexed peptides to a strictly linear mode of binding. This is common for all class II allotypes. The second component determining the binding affinity of a peptide is variable due to certain positions of polymorphism within class II binding sites. Different allotypes form different complementary pockets within the groove, thereby accounting for subtype-dependent selection of peptides, or specificity. Importantly, the constraints on the amino acid residues held within class II pockets are in general “softer” than for class I. There is much more cross reactivity of peptides among different HLA class II allotypes. The sequence of the +/−9 amino acids (i.e. 8, 9 or 10) of an MHC class II T cell epitope that fit in the groove of the MHC II molecule are usually numbered P1 to P9. Additional amino acids N-terminal of the epitope are numbered P-1, P-2 and so on, amino acids C-terminal of the epitope are numbered P+1, P+2 and so on.

The term “NKT cell epitope” refers to a part of an antigenic protein that is specifically recognized and bound by a receptor at the cell surface of an NKT cell and typically has a length of 7 amino acids. In particular, an NKT cell epitope is an epitope bound by CD1d molecules. The NKT cell epitope has a general motif [FWYHT]-X(2)-[VILM]-X(2)-[FWYHT]. Alternative versions of this general motif have at position 1 and/or position 7 the alternatives [FWYH], thus [FWYH]-X(2)-[VILM]-X(2)-[FWYH].

Alternative versions of this general motif have at position 1 and/or position 7 the alternatives [FWYT], [FWYT]-X(2)-[VILM]-X(2)-[FWYT]. Alternative versions of this general motif have at position 1 and/or position 7 the alternatives [FWY], [FWY]-X(2)-[VILM]-X(2)-[FWY].

Regardless of the amino acids at position 1 and/or 7, alternative versions of the general motif have at position 4 the alternatives [ILM], e.g. [FWYH]-X(2)-[ILM]-X(2)-[FWYH] or [FWYHT]-X(2)-[ILM]-X(2)-[FWYHT] or [FWY]-X(2)-[ILM]-X(2)-[FWY].

A CD1d binding motif in a protein can be identified by scanning a sequence for the above sequence motifs, either by hand, either by using an algorithm such as ScanProsite De Castro E. et al. (2006) Nucleic Acids Res. 34(Web Server issue):W362-W365.

“Natural killer T” or “NKT” cells constitute a distinct subset of non-conventional T lymphocytes that recognize antigens presented by the non-classical MHC complex molecule CD1d. Two subsets of NKT cells are presently described. Type I NKT cells, also called invariant NKT cells (iNKT), are the most abundant. They are characterized by the presence of an alpha-beta T cell receptor (TCR) made of an invariant alpha chain, Valphal4 in the mouse and Valpha24 in humans. This alpha chain is associated to a variable though limited number of beta chains. Type 2 NKT cells have an alpha-beta TCR but with a polymorphic alpha chain. However, it is apparent that other subsets of NKT cells exist, the phenotype of which is still incompletely defined, but which share the characteristics of being activated by glycolipids presented in the context of the CD1d molecule.

NKT cells typically express a combination of natural killer (NK) cell receptor, including NKG2D and NK1.1. NKT cells are part of the innate immune system, which can be distinguished from the adaptive immune system by the fact that they do not require expansion before acquiring full effector capacity. Most of their mediators are preformed and do not require transcription. NKT cells have been shown to be major participants in the immune response against intracellular pathogens and tumor rejection. Their role in the control of autoimmune diseases and of transplantation rejection is also advocated.

The recognition unit, the CD1d molecule, has a structure closely resembling that of the MHC class I molecule, including the presence of beta-2 microglobulin. It is characterized by a deep deft bordered by two alpha chains and containing highly hydrophobic residues, which accepts lipid chains. The deft is open at both extremities, allowing it to accommodate longer chains. The canonical ligand for CD1d is the synthetic alpha galactosylceramide (alpha GalCer). However, many natural alternative ligands have been described, including glyco- and phospholipids, the natural lipid sulfatide found in myelin, microbial phosphoinositol mannoside and alpha-glucuronosylceramide. The present consensus in the art (Matsuda et al (2008), Curr. Opinion Immunol., 20 358-368; Godfrey et al (2010), Nature rev. Immuno/11, 197-206) is still that CD1d binds only ligands containing lipid chains, or in general a common structure made of a lipid tail which is buried into CD1d and a sugar residue head group that protrudes out of CD1d.

The term “homologue” as used herein with reference to the epitopes used in the context of the invention, refers to molecules having at least 50%, at least 70%, at least 80%, at least 90%, at least 95% or at least 98% amino acid sequence identity with the naturally occurring epitope, thereby maintaining the ability of the epitope to bind an antibody or cell surface receptor of a B and/or T cell. Particular homologues of an epitope correspond to the natural epitope modified in at most three, more particularly in at most 2, most particularly in one amino acid.

The term “derivative” as used herein with reference to the peptides of the invention refers to molecules which contain at least the peptide active portion (i.e. the oxidoreductase motif and the MHC class II epitope capable of eliciting cytolytic CD4+ T cell activity) and, in addition thereto comprises a complementary portion which can have different purposes such as stabilising the peptides or altering the pharmacokinetic or pharmacodynamic properties of the peptide.

The term “sequence identity” of two sequences as used herein relates to the number of positions with identical nucleotides or amino acids divided by the number of nucleotides or amino acids in the shorter of the sequences, when the two sequences are aligned. In particular, the sequence identity is from 70% to 80%, from 81% to 85%, from 86% to 90%, from 91% to 95%, from 96% to 100%, or 100%.

The terms “peptide-encoding polynucleotide (or nucleic acid)” and “polynucleotide (or nucleic acid) encoding peptide” as used herein refer to a nucleotide sequence, which, when expressed in an appropriate environment, results in the generation of the relevant peptide sequence or a derivative or homologue thereof. Such polynucleotides or nucleic acids include the normal sequences encoding the peptide, as well as derivatives and fragments of these nucleic acids capable of expressing a peptide with the required activity. The nucleic acid encoding a peptide according to the invention or fragment thereof is a sequence encoding the peptide or fragment thereof originating from a mammal or corresponding to a mammalian, most particularly a human peptide fragment.

The term “oxidoreductase motif”, “thiol-oxidoreductase motif”, “thioreductase motif”, “thioredox motif” or “redox motif” are used herein as synonymous terms and refers to motifs involved in the transfer of electrons from one molecule (the reductant, also called the hydrogen or electron donor) to another (the oxidant, also called the hydrogen or electron acceptor). In particular, the term “oxidoreductase motif” can refer to the known [CST]XXC or CXX[CST] motifs, but particularly refers to the sequence motif [CST]XₙC or CXₙ[CST], wherein n is an integer selected from the group comprising: 0, 1, 3, 4, 5 or 6, and in which C stands for cysteine, S for serine, T for threonine and X for any amino acid. In order to have reducing activity, the cysteines present in a modified oxidoreductase motif should not occur as part of a cystine disulfide bridge.

The term “basic amino acid” refers to any amino acid that acts like a Bronsted-Lowry and Lewis base, and includes natural basic amino acids such as Arginine (R), Lysine (K) or Histidine (H), or non-natural basic amino acids, such as, but not limited to:
lysine variants like Fmoc-β-Lys(Boc)-OH (CAS Number 219967-68-7), Fmoc-Om(Boc)-OH also called L-omithine or omithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)OH(DiMe)-OH (CAS Number 441020-33-3);
tyrosine/phenylalanine variants like Fmoc-L-3Pal-OH (CAS Number 175453-07-3), Fmoc-β-HomoPhe(CN)-OH (CAS Number 270065-87-7), Fmoc-L-β-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0).

The immunogenic peptides disclosed herein typically can be of use in treating diseases caused by an elevated or uncontrolled immune response towards an allergen or (auto)antigen. Typically said antigenic protein is an auto-antigen, a soluble allofactor, an alloantigen shed by the graft, an antigen of an intracellular pathogen, an antigen of a viral vector used for gene therapy or gene vaccination, a tumor-associated antigen or an allergen. More preferably, said antigenic protein is an autoantigen involved in type-1 diabetes (T1D), a demyelinating disorder such as multiple sclerosis (MS) or neuromyelitis optica (NMO), or rheumatoid arthritis (RA).

The term “immune disorders” or “immune diseases” refers to diseases wherein a reaction of the immune system is responsible for or sustains a malfunction or non-physiological situation in an organism. Included in immune disorders are, inter alia, allergic disorders and autoimmune diseases.

The terms “allergic diseases” or “allergic disorders” as used herein refer to diseases characterised by hypersensitivity reactions of the immune system to specific substances called allergens (such as pollen, stings, drugs, or food). Allergy is the ensemble of signs and symptoms observed whenever an atopic individual patient encounters an allergen to which he has been sensitised, which may result in the development of various diseases, in particular respiratory diseases and symptoms such as bronchial asthma. Various types of classifications exist and mostly allergic disorders have different names depending upon where in the mammalian body it occurs. “Hypersensitivity” is an undesirable (damaging, discomfort-producing and sometimes fatal) reaction produced in an individual upon exposure to an antigen to which it has become sensitised; “Immediate hypersensitivity” depends of the production of IgE antibodies and is therefore equivalent to allergy.

The terms “autoimmune disease” or “autoimmune disorder” refer to diseases that result from an aberrant immune response of an organism against its own cells and tissues due to a failure of the organism to recognise its own constituent parts (down to the sub-molecular level) as “self”. The group of diseases can be divided in two categories, organ-specific and systemic diseases.

An “allergen” is defined as a substance, usually a macromolecule or a proteic composition which elicits the production of IgE antibodies in predisposed, particularly genetically disposed, individuals (atopics) patients. Similar definitions are presented in Liebers et al. (1996) Clin. Exp. Allergy 26, 494-516.

The term “demyelination” as used herein refers to damaging and/or degradation of myelin sheaths that surround axons of neurons which has as a consequence the formation of lesions or plaques. It is understood that the myelin acts as a protective covering surrounding nerve fibers in brain, optic nerves, and spinal cord. Due to demyelination, the signal conduction along the affected nerves is impaired (i.e. slowed or stopped), and may cause neurological symptoms such as deficiencies in sensation, movement, cognition, and/or other neurological function. The concrete symptoms a patient suffering from a demyelinating disease will vary depending on the disease and disease progression state. These may include a blurred and/or double vision, ataxia, clonus, dysarthria, fatigue, clumsiness, hand paralysis, hemiparesis, genital anaesthesia, incoordination, paresthesiasparaesthesia, ocular paralysis, impaired muscle coordination, muscle weakness, loss of sensation, impaired vision, neurological symptoms, unsteady way of walking (gait), spastic paraparesis, incontinence, hearing problems, speech problems, and others.

Therefore, “demyelinating diseases” or “demyelinating disorders” as used herein and commonly used in the art is indicative for any pathologic condition of the nervous system which involves impairment, for example damaging, or the myelin sheath of neurons. Demyelinating diseases may be stratified into central nervous system demyelinating diseases and peripheral nervous system. Alternatively, demyelinating diseases may be classified according to the cause of demyelination: destruction of myelin (demyelinating myelinoclastic), or abnormal and degenerative myelin (dysmyelinating leukodystrophic). Non-limiting examples of demyelinating diseases are Multiple Sclerosis (MS—(e.g. Relapsing/Remitting Multiple Sclerosis, Secondary Progressive Multiple Sclerosis, Progressive Relapsing Multiple Sclerosis, Primary Progressive Multiple Sclerosis, and Acute Fulminant Multiple Sclerosis), Neuromyelitis Optica (NMO), Optic Neuritis, Acute Disseminated Encephalomyelitis, Balo's Disease, HTLV-I Associated Myelopathy, Schilder's Disease, Transverse Myelitis, Idiopathic inflammatory demyelinating diseases, vitamin B12-induced central nervous system neuropathies, Central pontine myelinolysis, Myelopathies including tabes dorsalis, Leukodystrophies such as Adrenoleukodystrophy, Leukoencephalopathies such as Progressive multifocal leukoencephalopathy (PML), and Rubella induced mental retardation. It is appreciated by a skilled person that several of the above mentioned annotations are general classification names indicative of a group of diseases characterized be an identical or similar set of aberrant processes at the molecular level and/or an identical or similar set of (clinical) symptoms. A human patient having a demyelinating disorder can have one or more symptoms of a demyelinating disorder such as, but not limited to, impaired vision, numbness, weakness in extremities, tremors or spasticity, heat intolerance, speech impairment, incontinence, dizziness, or impaired proprioception (e.g., balance, coordination, sense of limb position). A human (e.g., a human patient) with a family history of a demyelinating disorder (e.g., a genetic predisposition for a demyelinating disorder), or who exhibits mild or infrequent symptoms of a demyelinating disorder described above can be, for the purposes of the method, considered at risk of developing a demyelinating disorder (e.g., Multiple Sclerosis). Preferred demyelinating diseases in the context of the current disclosure are those caused by MOG autoantigens or involving anti-MOG antibodies, including but not limited to Multiple Sclerosis (MS) or Neuromyelitis Optica (NMO).

The term “Multiple Sclerosis”, abbreviated herein and in the art as “MS”, indicates an autoimmune disorder affecting the central nervous system. MS is considered the most common non-traumatic disabling disease in young adults (Dobson and Giovannoni, (2019) Eur. J. Neurol. 26(1), 27-40), and the most common autoimmune disorder affecting the central nervous system (Berer and Krishnamoorthy (2014) FEBS Lett. 588(22), 4207-4213). MS may manifest itself in a subject by a large number of different symptoms ranging from physical over mental to psychiatric problems. Typical symptoms include blurred or double vision, muscle weakness, blindness in one eye, and difficulties in coordination and sensation. In most cases, MS may be viewed as a two-stage disease, with early inflammation responsible for relapsing-remitting disease and delayed neurodegeneration causing non-relapsing progression, i.e. secondary and primary progressive MS. Although progress is being made in the field, a conclusive underlying cause of the disease remains hitherto elusive and over 150 single nucleotide polymorphisms have been associated with MS susceptibility (International Multiple Sclerosis Genetics Consortium Nat Genet. (2013). 45(11):1353-60). Vitamin D deficiency, smoking, ultraviolet B (UVB) exposure, childhood obesity and infection by Epstein-Barr virus have been reported to contribute to disease development (Ascherio (2013) Expert Rev Neurother. 13(12 Suppl), 3-9).

Hence, MS can be regarded as a single diseases existing within a spectrum extending from relapsing (wherein inflammation is the dominant feature) to progressive (neurodegeneration dominant). Therefore it is evident that the term Multiple sclerosis as used herein encompasses any type of Multiple Sclerosis belonging to any kind of disease course classification. In particular the invention is envisaged to be a potent treatment strategy patient diagnosed with, or suspected of having clinically Isolated Syndrome (CIS), relapse-remitting MS (RRMS), secondary progressive MS (SPMS), primary progressive MS (PPMS), and even MS-suspected radiology isolated syndrome (RIS). While strictly not considered a disease course of MS, RIS is used to classify subjects showing abnormalities on the Magnetic Resonance Imaging (MRI) of brain and/or spinal cord that correspond to MS lesions and cannot be prima fade explained by other diagnoses. CIS is a first episode (by definition lasting for over 24 hours) of neurologic symptoms caused by inflammation and demyelination in the central nervous system. In accordance with RIS, CIS classified subjects may or may not continue to develop MS, with subjects showing MS-like lesions on a brain MRI more likely to develop MS. RRMS is the most common disease course of MS with 85% of subjects having MS being diagnosed with RRMS. RRMS diagnosed patients are a preferred group of patients in view of the current invention. RRMS is characterized by attacks of new or increasing neurologic symptoms, alternatively worded relapses or exacerbations. In RRMS, said relapses are followed by periods or partial or complete remission of the symptoms, and no disease progression is experienced and/or observed in these periods of remission. RRMS may be further classified as active RRMS (relapses and/or evidence of new MRI activity), non-active RRMS, worsening RRMS (increasing disability over a specified period of time after a relapse, or not worsening RRMS. A portion of RRMS diagnosed subject will progress to the SPMS disease course, which is characterized by a progressive worsening of neurologic function, i.e. an accumulation of disability, over time. SPMS subclassifications can be made such as active (relapses and/or new MRI activity), not active, progressive (disease worsening over time), or non-progressive SPMS. Finally, PPMS is an MS disease course characterized by worsening of neurologic function and hence an accumulation of disability from the onset of symptoms, without early relapse or remission. Further PPMS subgroups can be formed such as active PPMS (occasional relapse and/or new MRI activity), non-active PPMS, progressive PPMS (evidence of disease worsening over time, regardless of new MRI activity) and non-progressive PPMS.

In general, MS disease courses are characterized by substantial intersubject variability in terms of relapse and remission periods, both in severity (in case of relapse) and duration.

Several disease modifying therapies are available for MS, and therefore the current invention may be used as alternative treatment strategy, or in combination with these existing therapies. Non-limiting examples of active pharmaceutical ingredients include interferon beta-1a, interferon beta-1b, glatiramer acetate, glatiramer acetate, peginterferon beta-1a, teriflunomide, fingolimod, cladribine, siponimod, dimethyl fumarate, diroximel fumarate, ozanimod, alemtuzumab, mitoxantrone, ocrelizumab, and natalizumab. Alternatively, the invention may be used in combination with a treatment or medication aiming to relapse management, such as but not limited to methylprednisolone, prednisone, and adrenocorticotropic hormone(s) (ACM). Further, the invention may be used in combination with a therapy aiming to alleviate specific symptoms. Non-limiting examples include medications aiming to improve or avoid symptoms selected from the group consisting of: bladder problems, bowel dysfunction, depression, dizziness, vertigo, emotional changes, fatigue, itching, pain, sexual problems, spasticity, tremors, and walking difficulties.

MS is characterized by three intertwined hallmark characteristics: 1) lesion formation in the central nervous system, 2) inflammation, and 3) degradation of myelin sheaths of neurons. Despite traditionally being considered a demyelinating disease of the central nervous system and white matter, more recently reports have surfaced that demyelination of the cortical and deep gray matter may exceed white matter demyelination (Kutzelnigg et al. (2005). Brain. 128(11), 2705-2712). Two main hypotheses have been postulated as to how MS is caused at the molecular level. The commonly accepted “outside-in hypothesis” is based on the activation of peripheral autoreactive effector CD4+ T cells which migrate to the central nervous system and initiate the disease process. Once in the central nervous system, said T cells are locally reactivated by APCs and recruit additional T cells and macrophages to establish inflammatory lesions. Noteworthy, MS lesions have been shown to contain CD8+ T cells predominantly found at the lesion edges, and CD4+ T cells found more central in the lesions. These cells are thought to cause demyelination, oligodendrocyte destruction, and axonal damage, leading to neurologic dysfunction. Additionally, immune-modulatory networks are triggered to limit inflammation and to initiate repair, which results in at least partial remyelination reflected by clinical remission. Nonetheless, without adequate treatment, further attacks often lead to progression of the disease.

MS onset is believed to originate well before the first clinical symptoms are detected, as evidenced by the typical occurrence of apparent older and inactive lesions on the MRI of patients. Due to advances in the development of diagnostic methods, MS can now be detected even before a clinical manifestation of the disease (i.e. pre-symptomatic MS). In the context of the invention, “treatment of MS” and similar expressions envisage treatment of, and treatment strategies for, both symptomatic and pre-symptomatic MS. In particular, when the immunogenic peptides and/or resulting cytolytic CD4+ T cells are used for treating a pre-symptomatic MS patient, the disease is halted at such an early stage that clinical manifestations may be partially, or even completely avoided. MS wherein the subject is not fully responsive to a treatment of interferon beta is also encompassed within the term “MS”. The main antigens attacked by the immune system and leading to the disease are: Myelin oligodendrocyte glycoprotein (MOG), Myelin basic protein (MBP), Proteolipid protein (PLP), Myelin-oligodendrocytic basic protein (MOBP), and Oligodendrocyte-specific protein (OSP).

The term “Neuromyelitls Optica” or “NMO” and “NMO Spectrum Disorder (NMOSD)”, also known as “Devic's disease”, refers to an autoimmune disorder in which white blood cells and antibodies primarily attack the optic nerves and the spinal cord, but may also attack the brain (reviewed in Wingerchuk 2006, Int MS J. 2006 May; 13(2):42-50). The damage to the optic nerves produces swelling and inflammation that cause pain and loss of vision; the damage to the spinal cord causes weakness or paralysis in the legs or arms, loss of sensation, and problems with bladder and bowel function. NMO is a relapsing-remitting disease. During a relapse, new damage to the optic nerves and/or spinal cord can lead to accumulating disability. Unlike MS, there is no progressive phase of this disease. Therefore, preventing attacks is critical to a good long-term outcome. In cases associated with anti-MOG antibodies, it is considered that anti-MOG antibodies may trigger an attack on the myelin sheath resulting in demyelination. The cause of NMO in the majority of cases is due to a specific attack on auto-antigens. Up to a third of subjects may be positive for auto-antibodies directed against a component of myelin called myelin oligodendrocyte glycoprotein (MOG). People with anti-MOG related NMO similarly have episodes of transverse myelitis and optic neuritis.

The term “Rheumatoid Arthritis” or “RA” is an autoimmune, inflammatory disease that causes pain, swelling, stiffness, and loss of function in various joints (most commonly in the hands, wrists, and knees). The respective joint's lining becomes inflamed, leading to tissue damage, as well as chronic pain, unsteadiness, and deformity. There is generally a bilateral/symmetrical pattern of disease progression (e.g., both hands or both knees are affected). RA can also affect extra-articular sites, including the eyes, mouth, lungs, and heart. Patients can experience an acute worsening of their symptoms (called a flare) but with early intervention and appropriate treatment, symptoms can be ameliorated for a certain duration (reviewed by Sana Iqbal et al., 2019, US Pharm. 2019; 44(1)(Specialty&Oncology suppl):8-11). The antigens attacked by the immune system and responsible for the disease are diverse but some examples are: GRP78, HSP60, 60 kDa chaperonin 2, Gelsolin, Chitinase-3-like protein 1, Cathepsin S, Serum albumin, and Cathepsin D.

The term “type 1 diabetes” (T1D) or “diabetes type 1” (also known as “type 1 diabetes mellitus” or “immune mediated diabetes” or formerly known as “juvenile onset diabetes” or “insulin dependent diabetes”) is an autoimmune disorder that typically develops in susceptible individuals during childhood. At the basis of T1D pathogenesis is the destruction of most insulin-producing pancreatic beta-cells by an autoimmune mechanism. In short, the organism loses the immune tolerance towards the pancreatic beta-cells in charge of insulin production and induces an immune response, mainly cell-mediated, associated to the production of autoantibodies, which leads to the self-destruction of beta-cells. The main antigen attacked by the immune system and responsible for the disease is (pro)insulin but other examples are: GAD65, GAD67, IA-2 (ICA512), IA-2 (beta/phogrin), IGRP, Chromogranin, ZnT8 and HSP-60.

The term “therapeutically effective amount” refers to an amount of the peptide of the invention or derivative thereof, which produces the desired therapeutic or preventive effect in a patient. For example, in reference to a disease or disorder, it is the amount which reduces to some extent one or more symptoms of the disease or disorder, and more particularly returns to normal, either partially or completely, the physiological or biochemical parameters associated with or causative of the disease or disorder. Typically, the therapeutically effective amount is the amount of the peptide of the invention or derivative thereof, which will lead to an improvement or restoration of the normal physiological situation. For instance, when used to therapeutically treat a mammal affected by an immune disorder, it is a daily amount peptide/kg body weight of the said mammal. Alternatively, where the administration is through gene-therapy, the amount of naked DNA or viral vectors is adjusted to ensure the local production of the relevant dosage of the peptide of the invention, derivative or homologue thereof.

The term “natural” when referring to a peptide relates to the fact that the sequence is identical to a fragment of a naturally occurring protein (wild type or mutant). In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence.

In this context, it is realised that peptide fragments are generated from antigens, typically in the context of epitope scanning. By coincidence such peptides may comprise in their sequence a T cell epitope (an MHC class II epitope or a CD1d binding epitope) and in their proximity a sequence with the modified oxidoreductase motif as defined herein. Alternatively there can be an amino acid sequence of at most 11 amino acids, at most 7 amino acids, at most 4 amino acids, at most 2 amino acids between said epitope and said oxidoreductase motif, or even 0 amino acids (in other words the epitope and oxidoreductase motif sequence are immediately adjacent to each other). In preferred embodiment, such naturally occurring peptides comprising an oxidoreductase motif at a position of within at most 11 amino acids, at most 7 amino acids, at most 4 amino acids, at most 2 amino acids or even 0 amino acids of said T cell epitope in their wild-type or natural sequence, are disclaimed.

Amino acids are referred to herein with their full name, their three-letter abbreviation or their one letter abbreviation.

Motifs of amino acid sequences are written herein according to the format of Prosite. Motifs are used to describe a certain sequence variety at specific parts of a sequence. The symbol X is used for a position where any amino acid is accepted. Alternatives are indicated by listing the acceptable amino acids for a given position, between square brackets (‘[ ]’). For example: [CST] stands for an amino acid selected from Cys, Ser or Thr. Amino acids which are excluded as alternatives are indicated by listing them between curly brackets (‘{ }’). For example: {AM} stands for any amino acid except Ala and Met. The different elements in a motif are optionally separated from each other by a hyphen (-). In the context of the motifs disclosed in this specification, the disclosed general oxidoreductase motifs are typically accompanied by a hyphen not forming a connection with a different element outside the motif. These ‘open’ hyphens indicate the position of the physical connection of the motif with another portion of the immunogenic peptide such as a linker sequence or an epitope sequence. For example, a motif of the form “Zₘ-C-Xₙ-[CST]-” indicates that the [CST] is the amino acid connected to the other portion of the immunogenic peptide, and Z is a terminal amino acid of the immunogenic peptide. Preferred physical connections are peptide bonds. Repetition of an identical element within a motif can be indicated by placing behind that element a numerical value or a numerical range between parentheses. In this respect, “Xₙ” refers to n-times “X”. For example X(2) corresponds to X-X or XX; X(2, 5) corresponds to 2, 3, 4 or 5 X amino acids, A(3) corresponds to A-A-A or AAA. To distinguish between the amino acids, those outside the oxidoreductase motif can be called external amino acids, those within the oxidoreductase motif are called internal amino acids. Unless stated otherwise X represents any amino acid, particularly an L-amino acid, more particularly one of the 20 naturally occurring L-amino acids.

A peptide, comprising a T cell epitope, e.g. an MHC class II T-cell epitope or an NKT-cell epitope (or CD1d binding peptide epitope) and a modified peptide motif sequence, having reducing activity is capable of generating a population of antigen-specific cytolytic CD4+ T-cells, respectively cytolytic NKT-cells towards antigen-presenting cells.

Accordingly, in its broadest sense, the invention relates to peptides which comprise at least one T-cell epitope (MHC class II T-cell epitope or an NKT-cell epitope) of an antigen (self or non-self) with a potential to trigger an immune reaction, and a modified oxidoreductase sequence motif with a reducing activity on peptide disulfide bonds. The T cell epitope and the modified oxidoreductase motif sequence may be immediately adjacent to each other in the peptide or optionally separated by one or more amino acids (so called linker sequence). Optionally the peptide additionally comprises an endosome targeting sequence and/or additional “flanking” sequences.

The peptides of the invention comprise a T-cell epitope of an antigen (self or non self) with a potential to trigger an immune reaction, and a modified oxidoreductase motif. The reducing activity of the motif sequence in the peptide can be assayed for its ability to reduce a sulfhydryl group such as in the insulin solubility assay wherein the solubility of insulin is altered upon reduction, or with a fluorescence-labelled substrate such as insulin. An example of such assay uses a fluorescent peptide and is described in Tomazzolli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again.

The modified oxidoreductase motif may be positioned at the amino-terminus side of the T-cell epitope or at the carboxy-terminus of the T-cell epitope.

Peptide fragments with reducing activity are encountered in thioreductases which are small disulfide reducing enzymes including glutaredoxins, nucleoredoxins, thioredoxins and other thiol/disulfide oxidoreductases (Holmgren (2000) Antioxid. Redox Signal. 2, 811-820; Jacquot et al. (2002) Biochem. Pharm. 64, 1065-1069). They are multifunctional, ubiquitous and found in many prokaryotes and eukaryotes. They are known to exert reducing activity for disulfide bonds on proteins (such as enzymes) through redox active cysteines within conserved active domain consensus sequences well-known from e.g. Fomenko et al. ((2003) Biochemistry 42, 11214-11225; Fomenko et al. (2002) Prof. Science 11, 2285-2296), in which X stands for any amino acid. and WO2008/017517 comprising a cysteine at position 1 and/or 4. Thus the motif there is either CXX[CST] or [CST]XXC. Such domains are also found in larger proteins such as protein disulfide isomerase (PDI) and phosphoinositide-specific phospholipase C. The present invention has redesigned said motifs in search for more potency and activity.

The terms “cysteine”, “C”, “serine”, “S”, and “threonine”, “T”, when used in the light of the amino acid residues present in the oxidoreductase motifs disclosed herein respectively refer to naturally occurring cysteine, serine or threonine amino acids. Unless explicitly stated differently, said terms hence exclude chemically modified cysteines, serines and threonines such as those modified so as to carry an acetyl, methyl, ethyl or propionyl group, either on the N-terminal amide of the amino acid residue of the motif or on the C-terminal carboxy group.

As explained in detail further on, the peptides of the present invention can be made by chemical synthesis, which allows the incorporation of non-natural amino acids.

In the peptides of the present invention comprising a modified oxidoreductase motif, the motif is located such that, when the epitope fits into the MHC groove, the motif remains outside of the MHC binding groove. The modified oxidoreductase motif is placed either immediately adjacent to the epitope sequence within the peptide [in other words a linker sequence of zero amino acids between motif and epitope], or is separated from the T cell epitope by a linker comprising an amino acid sequence of 5 amino acids or less. More particularly, the linker comprises 1, 2, 3, 4, or 5 amino acids. Specific embodiments are peptides with a 0, 1, 2 or 3 amino acid linker between epitope sequence and modified oxidoreductase motif sequence. Apart from a peptide linker, other organic compounds can be used as linker to link the parts of the peptide to each other (e.g. the modified oxidoreductase motif sequence to the T cell epitope sequence).

The peptides of the present invention can further comprise additional short amino acid sequences N or C-terminally of the sequence comprising the T cell epitope and the modified oxidoreductase motif. Such an amino acid sequence is generally referred to herein as a ‘flanking sequence’. A flanking sequence can be positioned between the epitope and an endosomal targeting sequence and/or between the modified oxidoreductase motif and an endosomal targeting sequence. In certain peptides, not comprising an endosomal targeting sequence, a short amino acid sequence may be present N and/or C terminally of the modified oxidoreductase motif and/or epitope sequence in the peptide. More particularly a flanking sequence is a sequence of between 1 and 7 amino acids, most particularly a sequence of 2 amino acids.

In certain embodiments of the present invention, peptides are provided comprising one epitope sequence and a modified oxidoreductase motif sequence. Alternatively, oxidoreductase motifs can be provided at both the N and the C terminus of the T cell epitope sequence.

Other variations envisaged for the peptides of the present invention include peptides which contain repeats of a T cell epitope sequence wherein each epitope sequence is preceded and/or followed by the modified oxidoreductase motif (e.g. repeats of “oxidoreductase motif-epitope” or repeats of “oxidoreductase motif-epitope-oxidoreductase motif”). Herein the oxidoreductase motifs can all have the same sequence but this is not obligatory.

Typically, the peptides of the present invention comprise only one T cell epitope. As described below a T cell epitope in a protein sequence can be identified by functional assays and/or one or more in silica prediction assays. The amino acids in a T cell epitope sequence are numbered according to their position in the binding groove of the MHC proteins or bind the CD1d molecule. An MHC class II T-cell epitope present within a peptide typically consists of between 7 and 30 amino acids, preferably between 9 and 30 amino acids, such as of between 9 and 25 amino acids, yet more particularly of between 9 and 16 amino acids, yet most particularly consists of 9, 10, 11, 12, 13, 14, 15 or 16 amino acids. An NKT cell epitope present within a peptide typically consists of between 7 and 30 amino acids, such as of between 7 and 25 amino acids, yet more particularly of between 7 and 16 amino acids, yet most particularly consists of 7, 8, 9, 10, 11, 12, 13, 14, 15 or 16 amino acids.

In a more particular embodiment, the T cell epitope consists of a sequence of 9, 10, or 11 amino acids. In a further particular embodiment, the T-cell epitope is an epitope, which is presented to T cells by MHC-class II molecules [MHC class II restricted T cell epitopes]. Typically, T cell epitope sequence refers to the octapeptide or more specifically nonapeptide sequence which fits into the deft of an MHC II protein.

In a more particular embodiment, the T cell epitope consists of a sequence of 7, 8, or 9 amino acids. In a further particular embodiment, the T-cell epitope is an epitope, which is presented by CD1d molecules [NKT cell epitopes]. Typically NKT cell epitope sequence refers to the 7 amino acid peptide sequence which binds to and is presented by the CD1d protein.

The T cell epitope of the peptides of the present invention can correspond either to a natural epitope sequence of a protein or can be a modified version thereof, provided the modified T cell epitope retains its ability to bind within the MHC deft or to bind the CD1d receptor, similar to the natural T cell epitope sequence. The modified T cell epitope can have the same binding affinity for the MHC protein or the CD1d receptor as the natural epitope, but can also have a lowered affinity. In particular, the binding affinity of the modified peptide is no less than 10-fold less than the original peptide, more particularly no less than 5 times less. Peptides of the present invention have a stabilising effect on protein complexes. Accordingly, the stabilising effect of the peptide-MHC or CD1d complex compensates for the lowered affinity of the modified epitope for the MHC or CD1d molecule.

The sequence comprising the T cell epitope and the reducing compound within the peptide can be further linked to an amino acid sequence (or another organic compound) that facilitates uptake of the peptide into late endosomes for processing and presentation within MHC class II determinants. The late endosome targeting is mediated by signals present in the cytoplasmic tail of proteins and corresponds to well-identified peptide motifs. The late endosome targeting sequences allow for processing and efficient presentation of the antigen-derived T cell epitope by MHC-class II molecules. Such endosomal targeting sequences are contained, for example, within the gp75 protein (Vijayasaradhi et al. (1995) J. Cell. Biol. 130, 807-820), the human CD3 gamma protein, the HLA-BM 11 (Copier et al. (1996) J. Immunol. 157, 1017-1027), the cytoplasmic tail of the DEC205 receptor (Mahnke et al. (2000) J. Cell Biol. 151, 673-683). Other examples of peptides which function as sorting signals to the endosome are disclosed in the review of Bonifacio and Traub (2003) Annu. Rev. Biochem. 72, 395-447. Alternatively, the sequence can be that of a subdominant or minor T cell epitope from a protein, which facilitates uptake in late endosome without overcoming the T cell response towards the antigen. The late endosome targeting sequence can be located either at the amino-terminal or at the carboxy-terminal end of the antigen derived peptide for efficient uptake and processing and can also be coupled through a flanking sequence, such as a peptide sequence of up to 10 amino adds. When using a minor T cell epitope for targeting purpose, the latter is typically located at the amino-terminal end of the antigen derived peptide.

Alternatively, the present invention relates to the production of peptides containing hydrophobic residues that confer the capacity to bind to the CD1d molecule. Upon administration, such peptides are taken up by APC, directed to the late endosome where they are loaded onto CD1d and presented at the surface of the APC. Said hydrophobic peptides being characterized by a motif corresponding to the general sequence [FWHY]-XX-[ILMV]-XX-[FWTHY] (SEQ ID NO: 112) or [FW]-XX-[ILMV]-XX-[FW] (SEQ ID NO: 113) in which positions P1 and P7 are occupied by hydrophobic residues such as phenylalanine (F) or tryptophan (W). P7 is however permissive in the sense that it accepts alternative hydrophobic residues to phenylalanine or tryptophan, such as threonine (T) or histidine (H). The P4 position is occupied by an aliphatic residue such as isoleucine (I), leucine (L) or methionine (M). The present invention relates to peptides made of hydrophobic residues which naturally constitute a CD1d binding motif. In some embodiment, amino acid residues of said motif are modified, usually by substitution with residues which increase the capacity to bind to 15 CD1d. In a specific embodiment motifs are modified to fit more closely with said general motif More particularly, peptides are produced to contain a F or W at position 7.

Accordingly, the present invention envisages peptides of antigenic proteins and their use in eliciting specific immune reactions. These peptides can either correspond to fragments of proteins which comprise, within their sequence i.e. a reducing compound and a T cell epitope separated by at most 10, preferably 7 amino adds or less. Alternatively, and for most antigenic proteins, the peptides of the invention are generated by coupling a reducing compound, more particularly a reducing modified oxidoreductase motif as described herein, N-terminally or C-terminally to a T cell epitope of the antigenic protein (either directly adjacent thereto or with a linker of at most 10, more particularly at most 7 amino adds). Moreover the T cell epitope sequence of the protein and/or the modified oxidoreductase motif can be modified and/or one or more flanking sequences and/or a targeting sequence can be introduced (or modified), compared to the naturally occurring sequence. Thus, depending on whether or not the features of the present invention can be found within the sequence of the antigenic protein of interest, the peptides of the present invention can comprise a sequence which is ‘artificial’ or ‘naturally occurring’.

The peptides of the present invention can vary substantially in length. The length of the peptides can vary from 9, 10 or 11 amino acids, i.e. consisting of an NKT cell or MHC class II T cell epitope of respectively 7, 8 or 9 amino acids, adjacent thereto the minimal oxidoreductase motif of 2 amino acids (CC), to up to 12, 13, 14, 15, 16, 17, 18, 19, 20, 25, 30, 35, 40, 45 or up to 50 amino acids.

In particular embodiments, the complete peptide consists of between 9 amino acids up 20, 25, 30, 40, 50, 75 or 100 amino acids. For example, a peptide may comprise an endosomal targeting sequence of 40 amino acids, a flanking sequence of about 2 amino acids, an oxidoreductase motif as described herein of between 2 and about 11 amino acids, a linker of 4 to 7 amino acids and a T cell epitope peptide of minimally 7, 8 or 9 amino acids. More particularly, where the reducing compound is a modified oxidoreductase motif as described herein, the length of the (artificial or natural) sequence comprising the epitope and modified oxidoreductase motif optionally connected by a linker (referred to herein as ‘epitope-modified oxidoreductase motif’ sequence), without the endosomal targeting sequence, is critical. The ‘epitope-modified oxidoreductase motif’ more particularly has a length of 9, 10, 11, 12, 13, 14, 15, 16, 17, 18 or 19 amino acids. Such peptides of 9, 10, 11, 12, 13 or 14 to 19 amino acids can optionally be coupled to an endosomal targeting signal of which the size is less critical. The late endosome targeting is mediated by signals present in the cytoplasmic tail of proteins and correspond to well-identified peptide motifs such as the dileucine-based [DE]XXXL[LI] (SEQ ID NO: 114) or DXXLL (SEQ ID NO: 115) motif (e.g. DXXXLL (SEQ ID NO: 117)), the tyrosine-based YXX0 (SEQ ID NO: 117) motif or the so-called acidic duster motif. The symbol 0 represents amino acid residues with a bulky hydrophobic side chain such as Phe, Tyr and Trp. The late endosome targeting sequences allow for processing and efficient presentation of the antigen-derived T cell epitope by MHC class II or CD1d molecules.

As detailed above, in particular embodiments, the peptides of the present invention comprise a reducing modified oxidoreductase motif as described herein linked to a T cell epitope sequence.

In further particular embodiments, the peptides of the invention are peptides comprising T cell epitopes which do not comprise an amino acid sequence with redox properties within their natural sequence.

However, in alternative embodiments, the T cell epitope may comprise any sequence of amino acids ensuring the binding of the epitope to the MHC deft or to the CD1d molecule.

Where an epitope of interest of an antigenic protein comprises a modified oxidoreductase motif such as described herein within its epitope sequence, the immunogenic peptides according to the present invention comprise the sequence of a modified oxidoreductase motif as described herein and/or of another reducing sequence coupled N- or C-terminally to the epitope sequence such that (contrary to the modified oxidoreductase motif present within the epitope, which is buried within the deft) the attached modified oxidoreductase motif can ensure the reducing activity.

Accordingly the T cell epitope and motif are immediately adjacent or separated from each other and do not overlap. To assess the concept of “immediately adjacent” or “separated”, the 7, 8 or 9 amino acid sequence which fits in the MHC deft or CD1d molecule is determined and the distance between this octapeptide or nonapeptide with the modified oxidoreductase motif is determined.

Generally, the peptides of the present invention are not natural (thus no fragments of proteins as such) but artificial peptides which contain, in addition to a T cell epitope, a modified oxidoreductase motif as described herein, whereby the modified oxidoreductase motif is immediately separated from the T cell epitope by a linker consisting of up to seven, most particularly up to four or up to 2 amino acids.

It has been shown that upon administration (i.e. injection) to a mammal of a peptide comprising an oxidoreductase motif and an MHC class II T-cell epitope (or a composition comprising such a peptide), the peptide elicits the activation of T cells recognising the antigen derived T cell epitope and provides an additional signal to the T cell through reduction of surface receptor. This supra-optimal activation results in T cells acquiring cytolytic properties for the cell presenting the T cell epitope, as well as suppressive properties on bystander T cells.

Additionally, it has been shown that upon administration (i.e. injection) to a mammal of a peptide comprising an oxidoreductase motif and an NKT-cell epitope (or a composition comprising such a peptide), the peptide elicits the activation of T cells recognising the antigen derived T cell epitope and provides an additional signal to the T cell through binding to the CD1d surface receptor. This activation results in NKT cells acquiring cytolytic properties for the cell presenting the T cell epitope.

In this way, the peptides or composition comprising the peptides described in the present invention, which contain an antigen-derived T cell epitope and, outside the epitope, a modified oxidoreductase motif can be used for direct immunisation of mammals, including human beings. The invention thus provides peptides of the invention or derivatives thereof, for use as a medicine. Accordingly, the present invention provides therapeutic methods which comprise administering one or more peptides according to the present invention to a patient in need thereof.

The present invention offers methods by which antigen-specific T cells endowed with cytolytic properties can be elicited by immunisation with small peptides. It has been found that peptides which contain (i) a sequence encoding a T cell epitope from an antigen and (ii) a consensus sequence with redox properties, and further optionally also comprising a sequence to facilitate the uptake of the peptide into late endosomes for efficient MHC-class II presentation or CD1d receptor binding, elicit cytolytic CD4+ T-cells or NKT cells respectively.

The immunogenic properties of the peptides of the present invention are of particular interest in the treatment and prevention of immune reactions.

Peptides described herein are used as medicament, more particularly used for the manufacture of a medicament for the prevention or treatment of an immune disorder in a mammal, more in particular in a human.

The present invention describes methods of treatment or prevention of an immune disorder of a mammal in need for such treatment or prevention, by using the peptides of the invention, homologues or derivatives thereof, the methods comprising the step of administering to said mammal suffering or at risk of an immune disorder a therapeutically effective amount of the peptides of the invention, homologues or derivatives thereof such as to reduce the symptoms of the immune disorder. The treatment of both humans and animals, such as, pets and farm animals is envisaged. In an embodiment the mammal to be treated is a human. The immune disorders referred to above are in a particular embodiment selected from allergic diseases and autoimmune diseases.

The peptides of the invention or the pharmaceutical composition comprising such as defined herein is preferably administered through sub-cutaneous or intramuscular administration. Preferably, the peptides or pharmaceutical compositions comprising such can be injected sub-cutaneously (SC) in the region of the lateral part of the upper arm, midway between the elbow and the shoulder. When two or more separate injections are needed, they can be administered concomitantly in both arms.

The peptide according to the invention or the pharmaceutical composition comprising such is administered in a therapeutically effective close. Exemplary but non-limiting dosage regimens are between 50 and 1500 μg, preferably between 100 and 1200 μg. More specific dosage schemes can be between 50 and 250 μg, between 250 and 450 μg or between 850 and 1300 μg, depending on the condition of the patient and severity of disease. Dosage regimen can comprise the administration in a single close or in 2, 3, 4, 5, or more closes, either simultaneously or consecutively.

In certain embodiments, the treatment can be repeated several times throughout the disease of the subject. Such consecutive treatments can be done daily, or with an intermission of 1 to 10 days, such as for example every 5 to 10 days such as about every 7 days.

Alternatively, said treatment can be repeated weekly, biweekly, monthly, bimonthly, or every three to four months.

Exemplary non-limiting administration schemes are the following:
A low close scheme comprising the SC administration of 50 μg of peptide in two separate injections of 25 μg each (100 μL each) followed by three consecutive injections of 25 μg of peptide as two separate injections of 12.5 μg each (50 μL each).
A medium close scheme comprising the SC administration of 150 μg of peptide in two separate injections of 75 μg each (300 μL each) followed by three consecutive administrations of 75 μg of peptide as two separate injections of 37.5 μg each (150 μL each).
A high close scheme comprising the SC administration of 450 μg of peptide in two separate injections of 225 μg each (900 μL each) followed by three consecutive administrations of 225 μg of peptide as two separate injections of 112.5 μg each (450 μL each).

Other exemplary non-limiting administration schemes are the following:
A close scheme comprising 6 SC administration 2 weeks apart of 450 μg of peptide in two separate injections of 225 μg each.
A close scheme comprising 6 SC administration 2 weeks apart SC of 1350 μg of peptide in two separate injections of 675 μg each.

A particularly but non-limiting dosage regimen of the immunogenic peptide as defined herein is between 50 and 1500 μg, preferably between 450 and 1500 μg. Dosage regimen can comprise the administration in a single close or in 2, 3, 4, 5, 6 or more doses, either simultaneously or consecutively. Said treatment with the immunogenic peptide can be done 1 to 6 times, such as 1 to 4 times, preferably every 5 to 9 days, such as about every 7 days.

An exemplary close scheme of an immunogenic peptide comprising a known oxidoreductase motif and an insulin T-cell epitope can be found on ClinicalTrials.gov under Identifier NCT03272269.

The present invention provides for immunogenic peptides comprising a new oxidoreductase motif and a T-cell epitope of an antigenic protein, optionally separated by a linker of between 0 and 7 amino acids.

Said new oxidoreductase motif is selected from the group comprising:

Zₘ-[CST]-Xₙ-C- or Zₘ-C-Xₙ-[CST]-,

wherein X or Z is any amino acid, preferably a basic amino acid, more preferably selected from the group comprising: K, H, R and a non-natural basic amino acid, preferably K or H, more preferably K;
wherein n is an integer selected from the group comprising: 0, 1, 3, 4, 5 or 6, preferably 0, 1 or 3, wherein m is an integer between 0 and 3, preferably 0 or 1;
wherein C, S and T respectively indicate a cysteine, serine and threonine amino acid residue and wherein [CST] indicates any one of C, S, or T can be selected, preferably a non-modified natural amino acid as defined herein;

In a preferred embodiment, said oxidoreductase motif is CC or CXC, wherein X can be any amino acid selected from the group consisting of: G, A, V, L, I, M, F, W, P, S, T, C, Y, N, Q, D, E, K, R, and H, or non-natural basic amino acids. Preferably, X in the CXC motif is any amino acid except for C, S, or T. In a specific embodiment, X in the CXC motif is a basic amino acid, such as H, K, or R, or a non-natural basic amino acid such as, but not limited to:
lysine variants like Fmoc-β-Lys(Boc)-OH (CAS Number 219967-68-7), Fmoc-Om(Boc)-OH also called L-omithine or omithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)OH(DiMe)-OH (CAS Number 441020-33-3);
tyrosine/phenylalanine variants like Fmoc-L-3Pal-OH (CAS Number 175453-07-3), Fmoc-β-HomoPhe(CN)-OH (CAS Number 270065-87-7), Fmoc-L-β-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0).

Specific examples of the CXC motif are: CHC, CKC, CRC, CGC, CAC, CVC, CLC, CIC, CMC, CFC, CWC, CPC, CSC, CTC, CYC, CNC, CQC, CDC, and CEC. Any one of these exemplary CXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein. Preferred examples of such motifs are: KCHC (SEQ ID NO: 13), KCKC (SEQ ID NO: 12), KCRC (SEQ ID NO: 11), or KCGC, KCAC, KCVC, KCLC, KCIC, KCMC, KCFC, KCWC, KCPC, KCSC, KCTC, KCYC, KCNC, KCQC, KCDC, KCEC (respectively SEQ ID NO: 141 to 156), or HCHC (SEQ ID NO: 19), HCKC (SEQ ID NO: 18), HCRC (SEQ ID NO: 19), or HCGC, HCAC, HCVC, HCLC, HCIC, HCMC, HCFC, HCWC, HCPC, HCSC HCTC, HCYC, HCNC, HCQC, HCDC, HCEC (respectively SEQ ID NO: 157 to 172), or RCHC (SEQ ID NO: 16), RCKC (SEQ ID NO: 15), RCRC (SEQ ID NO: 14), or RCGC, RCAC, RCVC, RCLC, RCIC, RCMC, RCFC, RCWC, RCPC, RCSC, RCTC, RCYC, RCNC, RCQC, RCDC, and RCEC (respectively SEQ ID NO: 173 to 188);

In a preferred embodiment, said oxidoreductase motif is CX₃C, i.e. CXXXC, typically CX¹X²X³C, wherein X¹, X², and X³, each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², and X³ in said motif is any amino acid except for C, S, or T. In a specific embodiment, at least one of X¹, X², or X³ in said motif is a basic amino acid, such as H, K, or R, or a non-natural basic amino acid as defined herein.

Specific examples of the CXXXC motif are: CXPYC (SEQ ID NO: 186), CPXYC (SEQ ID NO: 190), and CPYXC (SEQ ID NO: 191), wherein X can be can be any amino acid, more preferably CXPYC (SEQ ID NO: 186), such as: CKPYC (SEQ ID NO: 192), CRPYC (SEQ ID NO: 55), CHPYC (SEQ ID NO: 193), or CGPYC, CAPYC, CVPYC, CLPYC, CIPYC, CMPYC, CFPYC, CWPYC, CPPYC, CSPYC, CTPYC, CCPYC, CYPYC, CNPYC, CQPYC, CDPYC, and CEPYC (respectively SEQ ID NO: 194 to 210); or

CPXYC (SEQ ID NO: 190), such as: CPKYC, CPRYC, CPHYC, CPGYC, CPAYC, CPVYC, CPLYC, CPIYC, CPMYC, CPFYC, CPWYC, CPPYC, CPSYC, CPTYC, CPCYC, CPYYC, CPNYC, CPQYC, CPDYC, and CPEYC (respectively SEQ ID NO: 211 to 230); or CPYXC (SEQ ID NO: 191), such as: CPYKC, CPYRC, CPYHC, CPYGC, CPYAC, CPYVC, CPYLC, CPYIC, CPYMC, CPYFC, CPYWC, CPYPC, CPYSC, CPYTC, CPYCC, CPYYC, CPYNC, CPYQC, CPYDC, CPYEC, and CPYLC (respectively SEQ ID NO: 231 to 251).

Further specific examples of the CXXXC motif are: CXHGC (SEQ ID NO: 252), CHXGC (SEQ ID NO: 253), and CHGXC (SEQ ID NO: 254), wherein X can be can be any amino acid, more preferably CXHGC (SEQ ID NO: 252), such as: CKHGC, CRHGC, CHHGC, CGHGC, CAHGC, CVHGC, CLHGC, CIHGC, CMHGC, CFHGC, CWHGC, CPHGC, CSHGC, CTHGC, CCHGC, CYHGC, CNHGC, CQHGC, CDHGC, and CEHGC (respectively SEQ ID NO: 255 to 274); or

CGXHC (SEQ ID NO: 253), such as: CGKHC, CGRHC, CGHHC, CGGHC, CGAHC, CGVHC, CGLHC, CGIHC, CGMHC, CGFHC, CGWHC, CGPHC, CGSHC, CGTHC, CGCHC, CGYHC, CGNHC, CGQHC, CGDHC, and CGEHC (respectively SEQ ID NO: 275 to 294); or CHGXC (SEQ ID NO: 254), such as: CHGKC, CHGRC, CHGHC, CHGGC, CHGAC, CHGVC, CHGLC, CHGIC, CHGMC, CHGFC, CHGWC, CHGPC, CHGSC, CHGTC, CHGCC, CHGYC, CHGNC, CHGQC, CHGDC, and CHGEC (respectively SEQ ID NO: 295 to 314).

Further specific examples of the CXXXC motif are: CXGPC (SEQ ID NO: 315), CGXPC (SEQ ID NO: 316), and CGPXC (SEQ ID NO: 317), wherein X can be can be any amino acid, more preferably CXGPC (SEQ ID NO: 315), such as: CKGPC, CRGPC, CHGPC, CGGPC, CAGPC, CVGPC, CLGPC, CIGPC, CMGPC, CFGPC, CWGPC, CPGPC, CSGPC, CTGPC, CCGPC, CYGPC, CNGPC, CQGPC, CDGPC, and CEGPC (respectively SEQ ID NO: 318 to 337); or

CGXPC (SEQ ID NO: 316), such as: CGKPC, CGRPC, CGHPC, CGGPC, CGAPC, CGVPC, CGLPC, CGIPC, CGMPC, CGFPC, CGWPC, CGPPC, CGSPC, CGTPC, CGCPC, CGYPC, CGNPC, CGQPC, CGDPC, and CGEPC (respectively SEQ ID NO: 338 to 357); or CGPXC (SEQ ID NO: 317), such as: CGPKC, CGPRC, CGPHC, CGPGC, CGPAC, CGPVC, CGPLC, CGPIC, CGPMC, CGPFC, CGPWC, CGPPC, CGPSC, CGPTC, CGPCC, CGPYC, CGPNC, CGPQC, CGPDC, and CGPEC (respectively SEQ ID NO: 358 to 377).

Further specific examples of the CXXXC motif are: CXGHC (SEQ ID NO: 378), CGXHC (SEQ ID NO: 379), and CGHXC (SEQ ID NO: 380), wherein X can be can be any amino acid, more preferably CXGHC (SEQ ID NO: 378), such as: CKGHC, CRGHC, CHGHC, CGGHC, CAGHC, CVGHC, CLGHC, CIGHC, CMGHC, CFGHC, CWGHC, CPGHC, CSGHC, CTGHC, CCGHC, CYGHC, CNGHC, CQGHC, CDGHC, and CEGHC (respectively SEQ ID NO: 381 to 400); or

CGXHC (SEQ ID NO: 379), such as: CGKHC, CGRHC, CGHHC, CGGHC, CGAHC, CGVHC, CGLHC, CGIHC, CGMHC, CGFHC, CGWHC, CGPHC, CGSHC, CGTHC, CGCHC, CGYHC, CGNHC, CGQHC, CGDHC, and CGEHC (respectively SEQ ID NO: 659 to 678); or CGHXC (SEQ ID NO: 380), such as: CGHKC, CGHRC, CGHHC, CGHGC, CGHAC, CGHVC, CGHLC, CGHIC, CGHMC, CGHFC, CGHWC, CGHPC, CGHSC, CGHTC, CGHCC, CGHYC, CGHNC, CGHQC, CGHDC, and CGHEC (respectively SEQ ID NO: 421 to 439).

Further specific examples of the CXXXC motif are: CXGFC (SEQ ID NO: 440), CGXFC (SEQ ID NO: 401), and CGFXC (SEQ ID NO: 441), wherein X can be can be any amino acid, more preferably CXGFC (SEQ ID NO: 440), such as: CKGFC, CRGFC, CHGFC, CGGFC, CAGFC, CVGFC, CLGFC, CIGFC, CMGFC, CFGFC, CWGFC, CPGFC, CSGFC, CTGFC, CCGFC, CYGFC, CNGFC, CQGFC, CDGFC, and CEGFC (respectively SEQ ID NO: 442 to 460); or

CGXFC (SEQ ID NO: 401), such as: CGKFC, CGRFC, CGHFC, CGGFC, CGAFC, CGVFC, CGLFC, CGIFC, CGMFC, CGFFC, CGWFC, CGPFC, CGSFC, CGTFC, CGCFC, CGYFC, CGNFC, CGQFC, CGDFC, and CGEFC (respectively SEQ ID NO: 402 to 420); or CGFXC (SEQ ID NO: 441), such as: CGFKC, CGFRC, CGFHC, CGFGC, CGFAC, CGFVC, CGFLC, CGFIC, CGFMC, CGFFC, CGFWC, CGFPC, CGFSC, CGFTC, CGFCC, CGFYC, CGFNC, CGFQC, CGFDC, and CGFEC (respectively SEQ ID NO: 461 to 479).

Further specific examples of the CXXXC motif are: CXRLC (SEQ ID NO: 480), CRXLC (SEQ ID NO: 481), and CRLXC (SEQ ID NO: 482), wherein X can be can be any amino acid, more preferably CXRLC (SEQ ID NO: 480), such as: CKRLC, CRRLC, CHRLC, CGRLC, CARLC, CVRLC, CLRLC, CIRLC, CMRLC, CFRLC, CWRLC, CPRLC, CSRLC, CTRLC, CCRLC, CYRLC, CNRLC, CQRLC, CDRLC, and CERLC (respectively SEQ ID NO: 483 to 502); or

CRXLC (SEQ ID NO: 481), such as: CRKLC, CRRLC, CRHLC, CRGLC, CRALC, CRVLC, CRLLC, CRILC, CRMLC, CRFLC, CRWLC, CRPLC, CRSLC, CRTLC, CRCLC, CRYLC, CRNLC, CRQLC, CRDLC, and CRELC (respectively SEQ ID NO: 503 to 522); or
CRLXC (SEQ ID NO: 482), such as: CRLKC, CRLRC, CRLHC, CRLGC, CRLAC, CRLVC, CRLLC, CRLIC, CRLMC, CRLFC, CRLWC, CRLPC, CRLSC, CRLTC, CRLCC, CRLYC, CRLNC, CRLQC, CRLDC, and CRLEC (respectively SEQ ID NO: 523 to 541).

Further specific examples of the CXXXC motif are: CXHPC (SEQ ID NO: 542), CHXPC (SEQ ID NO: 543), and CHPXC (SEQ ID NO: 544), wherein X can be can be any amino acid, more preferably CXHPC (SEQ ID NO: 542), such as: CKHPC, CRHPC, CHHPC, CGHPC, CAHPC, CVHPC, CLHPC, CIHPC, CMHPC, CFHPC, CWHPC, CPHPC, CSHPC, CTHPC, CCHPC, CYHPC, CNHPC, CQHPC, CDHPC, and CEHPC (respectively SEQ ID NO: 545 to 564); or

CHXPC (SEQ ID NO: 543), such as: CHKPC, CHRPC, CHHPC, CHGPC, CHAPC, CHVPC, CHLPC, CHIPC, CHMPC, CHFPC, CHWPC, CHPPC, CHSPC, CHTPC, CHCPC, CHYPC, CHNPC, CHOPC, CHDPC, CHEPC, and CHLPC (respectively SEQ ID NO: 566 to 585); or CHPXC (SEQ ID NO: 544), such as: CHPKC, CHPRC, CHPHC, CHPGC, CHPAC, CHPVC, CHPLC, CHPIC, CHPMC, CHPFC, CHPWC, CHPPC, CHPSC, CHPTC, CHPCC, CHPYC, CHPNC, CHPQC, CHPDC, and CHPEC (respectively SEQ ID NO: 586 to 605).

Any one of these exemplary CXXXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

In a preferred embodiment, said oxidoreductase motif is CX₄C, i.e. C)X(XC (SEQ ID NO: 606), typically CX¹X²X³X⁴C, wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³ and X⁴ in said motif is any amino acid except for C, S, or T. In a specific embodiment, at least one of X¹, X², X³ or X⁴ in said motif is a basic amino acid, such as H, K, or R, or a non-natural basic amino acid as defined herein.

Specific examples of the CXXXXC motif are: CLAVLC, CTVQAC or CGAVHC (respectively SEQ ID NO: 607 to 609) and their variants such as: CX¹AVLC, CLX²VLC, CLAX³LC (respectively SEQ ID NO: 610 to 612), or CLAVX₄C; CX¹VQAC, CTX²QAC, CTVX³AC (respectively SEQ ID NO: 613 to 616), or CTVQX⁴C; CX¹AVHC, CGX²VHC, CGAX³HC (respectively SEQ ID NO: 617 to 620), or CGAVX⁴C (SEQ ID NO: 621); wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein.

Any one of these exemplary CXXXXC (SEQ ID NO: 622) motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

In a preferred embodiment, said oxidoreductase motif is CXSC, i.e. CXXXXXC, typically CX¹X²X³X⁴X⁵C, wherein X¹, X², X³, X⁴ and X⁵ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³, X⁴ and X⁵ in said motif is any amino acid except for C, S, or T. In a specific embodiment, at least one of X¹, X², X³ X⁴ or X⁵ in said motif is a basic amino acid, such as H, K, or R, or a non-natural basic amino acid as defined herein.

Specific examples of the CXXX(XC motif are: CPAFPLC (SEQ ID NO: 623) or CDQGGEC (SEQ ID NO: 624) and their variants such as: CX¹AFPLC, CPX²FPLC, CPAX³PLC, CPAFX⁴LC, or CPAFPX⁵C; CX¹QGGEC, CDX²GGEC, CDQX³GEC, CDQGX⁴EC, or CDQGGX⁵C (respectively SEQ ID NO: 625 to 634), wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural basic amino acids as defined herein. Any one of these exemplary CXX(XC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

In a preferred embodiment, said oxidoreductase motif is CX₆C, i.e. CXXXXXXC (SEQ ID NO: 635), typically CX¹X²X³X⁴X⁵X⁶C, wherein X¹, X², X³, X⁴X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³, X⁴, X⁵ and X⁶ in said motif is any amino acid except for C, S, or T. In a specific embodiment, at least one of X¹, X², X³ X⁴, X⁵ or X⁶ in said motif is a basic amino acid, such as H, K, or R, or a non-natural basic amino acid as defined herein.

A specific example of the CXXXXXXC motif is: CDIADKYC (SEQ ID NO: 636) or variants thereof such as: CX¹IADKYC, CDX²ADKYC, CDIX³DKYC, CDIAX⁴KYC, CDIADX⁵YC, or CDIADKX⁶C (respectively SEQ ID NO: 637 to 642), wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural basic amino acids as defined herein.

Any one of these exemplary CXXX(XC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

Particularly preferred examples of such oxidoreductase motifs are:

C[KHR]C, CX[KHR]XC, CXX[KHR]C, C[KHR]XXC, [KHR]CC, [KHR]CXC, [KHR]XXXC CC[KHR], CXC[KHR], CXXXC[KHR], [KHR]CC[KHR], [KHR]CXC[KHR], [KHR]CXXXC[KHR], [KHR]C[KHR]C, C[KHR]C[KHR], [KHR]CXX[KHR]C, [KHR]CX[KHR]XC, [KHR]C[KHR]XXC, CXX[KHR]C[KHR], CX[KHR]XC[KHR], C[KHR]XXC[KHR] (respectively SEQ ID NO: 643 to 658), and the like.

The peptides of the present invention can also be used in diagnostic in vitro methods for detecting class II restricted CD4+ T cells in a sample. In this method a sample is contacted with a complex of an MHC class II molecule and a peptide according to the present invention. The CD4+ T cells are detected by measuring the binding of the complex with cells in the sample, wherein the binding of the complex to a cell is indicative for the presence of CD4+ T cells in the sample. The complex can be a fusion protein of the peptide and an MHC class II molecule. Alternatively MHC molecules in the complex are tetramers. The complex can be provided as a soluble molecule or can be attached to a carrier.

The peptides of the present invention can also be used in diagnostic in vitro methods for detecting NKT cells in a sample. In this method a sample is contacted with a complex of a CD1d molecule and a peptide according to the present invention. The NKT cells are detected by measuring the binding of the complex with cells in the sample, wherein the binding of the complex to a cell is indicative for the presence of NKT cells in the sample.

The complex can be a fusion protein of the peptide and a CD1d molecule.

Accordingly, in particular embodiments, the methods of treatment and prevention of the present invention comprise the administration of an immunogenic peptide as described herein, wherein the peptide comprise a T cell epitope of an antigenic protein which plays a role in the disease to be treated (for instance such as those described above). In further particular embodiments, the epitope used is a dominant epitope.

Peptides in accordance of the present invention will be prepared by synthesising a peptide wherein T cell epitope and modified oxidoreductase motif will be separated by 0 to 7 amino acids. In certain embodiments the modified oxidoreductase motif can be obtained by introducing 1, 2 or 3 mutations outside the epitope sequence, to preserve the sequence context as occurring in the protein. Typically amino-acids in P-2 and P-1, as well as in P+10 and P+11, with reference to the nonapeptide which are part of the natural sequence are preserved in the peptide sequence. These flanking residues generally stabilize the binding to MHC class II or CD1d molecules. In other embodiments the sequence N terminal or C terminal of the epitope will be unrelated to the sequence of the antigenic protein containing the T cell epitope sequence.

Thus based upon the above methods for designing a peptide, a peptide is generated by chemical peptide synthesis, recombinant expression methods or in more exceptional cases, proteolytic or chemical fragmentation of proteins.

Peptides as produced in the above methods can be tested for the presence of a T cell epitope in in vitro and in vivo methods, and can be tested for their reducing activity in in vitro assays. As a final quality control, the peptides can be tested in in vitro assays to verify whether the peptides can generate CD4+ T or NKT cells which are cytolytic via an apoptotic pathway for antigen presenting cells presenting the antigen which contains the epitope sequence which is also present in the peptide with the modified oxidoreductase motif.

The peptides of the present invention can be generated using recombinant DNA techniques, in bacteria, yeast, insect cells, plant cells or mammalian cells. In view of the limited length of the peptides, they can be prepared by chemical peptide synthesis, wherein peptides are prepared by coupling the different amino acids to each other. Chemical synthesis is particularly suitable for the inclusion of e.g. D-amino acids, amino acids with non-naturally occurring side chains or natural amino acids with modified side chains, etc.

Chemical peptide synthesis methods are well described and peptides can be ordered from companies such as Applied Biosystems and other companies.

Peptide synthesis can be performed as either solid phase peptide synthesis (SPPS) or contrary to solution phase peptide synthesis. The best known SPPS methods are t-Boc and Fmoc solid phase chemistry:

During peptide synthesis several protecting groups are used. For example hydroxyl and carboxyl functionalities are protected by t-butyl group, lysine and tryptophan are protected by t-Boc group, and asparagine, glutamine, cysteine and histidine are protected by trityl group, and arginine is protected by the pbf group. If appropriate, such protecting groups can be left on the peptide after synthesis. Peptides can be linked to each other to form longer peptides using a ligation strategy (chemoselective coupling of two unprotected peptide fragments) as originally described by Kent (Schnelzer & Kent (1992) Int. J. Pept. Protein Res. 40, 180-193) and reviewed for example in Tam et al. (2001) Biopolymers 60, 194-205 provides the tremendous potential to achieve protein synthesis which is beyond the scope of SPPS. Many proteins with the size of 100-300 residues have been synthesised successfully by this method. Synthetic peptides have continued to play an ever increasing crucial role in the research fields of biochemistry, pharmacology, neurobiology, enzymology and molecular biology because of the enormous advances in the SPPS.

Alternatively, the peptides can be synthesised by using nucleic acid molecules which encode the peptides of this invention in an appropriate expression vector which include the encoding nucleotide sequences. Such DNA molecules may be readily prepared using an automated DNA synthesiser and the well-known codon-amino acid relationship of the genetic code. Such a DNA molecule also may be obtained as genomic DNA or as cDNA using oligonucleotide probes and conventional hybridisation methodologies. Such DNA molecules may be incorporated into expression vectors, including plasmids, which are adapted for the expression of the DNA and production of the polypeptide in a suitable host such as bacterium, e.g. Escherichia coli, yeast cell, animal cell or plant cell.

The physical and chemical properties of a peptide of interest (e.g. solubility, stability) are examined to determine whether the peptide is/would be suitable for use in therapeutic compositions. Typically this is optimised by adjusting the sequence of the peptide. Optionally, the peptide can be modified after synthesis (chemical modifications e.g. adding/deleting functional groups) using techniques known in the art.

The mechanism of action of immunogenic peptides comprising a standard oxidoreductase motif and an MHC class II T-cell epitope is substantiated with experimental data disclosed in the above cited PCT application WO2008/017517 and publications of the present inventors. The mechanism of action of immunogenic peptides comprising a standard oxidoreductase motif and a CD1d binding NKT-cell epitope is substantiated with experimental data disclosed in the above cited PCT application WO2012/069568 and publications of the present inventors.

The present invention provides methods for generating antigen-specific cytolytic CD4+ T-cells (when using an immunogenic peptide as disclosed herein comprising an MHC class II epitope), or antigen-specific cytolytic NKT-cells (when using an immunogenic peptide as disclosed herein comprising an NKT cell epitope binding the CD1d molecule) either in vivo or in vitro.

The present invention describes in vivo methods for the production of the antigen-specific CD4+ T cells or NKT cells. A particular embodiment relates to the method for producing or isolating the CD4+ T cells or NKT cells by immunising animals (including humans) with the peptides of the invention as described herein and then isolating the CD4+ T cells or NKT cells from the immunised animals. The present invention describes in vitro methods for the production of antigen specific cytolytic CD4+ T cells or NKT cells towards APC. The present invention provides methods for generating antigen specific cytolytic CD4+ T cells and NKT cells towards APC.

In one embodiment, methods are provided which comprise the isolation of peripheral blood cells, the stimulation of the cell population in vitro by an immunogenic peptide according to the invention and the expansion of the stimulated cell population, more particularly in the presence of IL-2. The methods according to the invention have the advantage a high number of CD4+ T cells is produced and that the CD4+ T cells can be generated which are specific for the antigenic protein (by using a peptide comprising an antigen-specific epitope).

In an alternative embodiment, the CD4+ T cells can be generated in vivo, i.e. by the injection of the immunogenic peptides described herein to a subject, and collection of the cytolytic CD4+ T cells generated in vivo.

The antigen-specific cytolytic CD4+ T cells towards APC, obtainable by the methods of the present invention are of particular interest for the administration to mammals for immunotherapy, in the prevention of allergic reactions and the treatment of auto-immune diseases. Both the use of allogenic and autogeneic cells are envisaged.

Cytolytic CD4+ T cells populations are obtained as described herein below.

In one embodiment, the invention provides ways to expand specific NKT cells, with as a consequence increased activity comprising, but not limited to:

(i) increased cytokine production
(ii) increased contact- and soluble factor-dependent elimination of antigen-presenting cells. The result is therefore a more efficient response towards intracellular pathogens, autoantigens, preferably autoantigens involved in type-1 diabetes (T1D), demyelinating disorders such as multiple sclerosis (MS) or neuromyelitis optica (NMO), or rheumatoid arthritis (RA).

The present invention also relates to the identification of NKT cells with required properties in body fluids or organs. The method comprises identification of NKT cells by virtue of their surface phenotype, including expression of NK1.1, CD4, NKG2D and CD244. Cells are then contacted with NKT cell epitopes defined as peptides able to be presented by the CD1d molecule. Cells are then expanded in vitro in the presence of IL-2 or IL-15 or IL-7.

Antigen-specific cytolytic CD4+ T cells or NKT cells as described herein can be used as a medicament, more particularly for use in adoptive cell therapy, more particularly in the treatment of acute allergic reactions and relapses of autoimmune diseases such as multiple sclerosis. Isolated cytolytic CD4+ T cells or NKT cells or cell populations, more particularly antigen-specific cytolytic CD4+ T cell or NKT cell populations generated as described are used for the manufacture of a medicament for the prevention or treatment of immune disorders. Methods of treatment by using the isolated or generated cytolytic CD4+ T cells or NKT cells are disclosed.

As explained in WO2008/017517 cytolytic CD4+ T cells towards APC can be distinguished from natural Treg cells based on expression characteristics of the cells. More particularly, a cytolytic CD4+ T cell population demonstrates one or more of the following characteristics compared to a natural Treg cell population:

an increased expression of surface markers including CD103, CTLA-4, Fasl and ICOS upon activation, intermediate expression of CD25, expression of CD4, ICOS, CTLA-4, GTR and low or no expression of CD127 (IL7-R), no expression of CD27, expression of transcription factor T-bet and egr-2 (Krox-20) but not of the transcription repressor Foxp3, a high production of IFN-gamma and no or only trace amounts of IL-10, IL-4, IL-5, IL-13 or TGF-beta.

Further the cytolytic T cells express CD45RO and/or CD45RA, do not express CCR7, CD27 and present high levels of granzyme B and other granzymes as well as Fas ligand.

As explained in WO2008/017517 cytolytic NKT cells against towards APC can be distinguished from non-cytolytic NKT cells based on expression characteristics of the cells. More particularly, a cytolytic CD4+NKT cell population demonstrates one or more of the following charactenstics compared to a non-cytolytic NKT cell population: expression of NK1.I, CD4, NKG2D and CD244.

The peptides of the invention will, upon administration to a living animal, typically a human being, elicit specific T cells exerting a suppressive activity on bystander T cells.

In specific embodiments the cytolytic cell populations of the present invention are characterised by the expression of FasL and/or Interferon gamma. In specific embodiments the cytolytic cell populations of the present invention are further characterised by the expression of GranzymeB.

This mechanism also implies and the experimental results show that the peptides of the invention, although comprising a specific T-cell epitope of a certain antigen, can be used for the prevention or treatment of disorders elicited by an immune reaction against other T-cell epitopes of the same antigen or in certain circumstances even for the treatment of disorders elicited by an immune reaction against other T-cell epitopes of other different antigens if they would be presented through the same mechanism by MHC class II molecules or CD1d molecules in the vicinity of T cells activated by peptides of the invention.

Isolated cell populations of the cell type having the characteristics described above, which, in addition are antigen-specific, i.e. capable of suppressing an antigen-specific immune response are disclosed.

The present invention provides pharmaceutical compositions comprising one or more peptides according to the present invention, further comprising a pharmaceutically acceptable carrier. As detailed above, the present invention also relates to the compositions for use as a medicine or to methods of treating a mammal of an immune disorder by using the composition and to the use of the compositions for the manufacture of a medicament for the prevention or treatment of immune disorders. The pharmaceutical composition could for example be a vaccine suitable for treating or preventing immune disorders, especially airborne and foodborne allergy, as well as diseases of allergic origin. As an example described further herein of a pharmaceutical composition, a peptide according to the invention is adsorbed on an adjuvant suitable for administration to mammals, such as aluminium hydroxide (alum). Typically, 50 μg of the peptide adsorbed on alum are injected by the subcutaneous route on 3 occasions at an interval of 2 weeks. It should be obvious for those skilled in the art that other routes of administration are possible, including oral, intranasal or intramuscular. Also, the number of injections and the amount injected can vary depending on the conditions to be treated. Further, other adjuvants than alum can be used, provided they facilitate peptide presentation in MHC-class II presentation and T cell activation. Thus, while it is possible for the active ingredients to be administered alone, they typically are presented as pharmaceutical formulations. The formulations, both for veterinary and for human use, of the present invention comprise at least one active ingredient, as above described, together with one or more pharmaceutically acceptable carriers. The present invention relates to pharmaceutical compositions, comprising, as an active ingredient, one or more peptides according to the invention, in admixture with a pharmaceutically acceptable carrier. The pharmaceutical composition of the present invention should comprise a therapeutically effective amount of the active ingredient, such as indicated hereinafter in respect to the method of treatment or prevention. Optionally, the composition further comprises other therapeutic ingredients. Suitable other therapeutic ingredients, as well as their usual dosage depending on the class to which they belong, are well known to those skilled in the art and can be selected from other known drugs used to treat immune disorders.

The term “pharmaceutically acceptable carrier” as used herein means any material or substance with which the active ingredient is formulated in order to facilitate its application or dissemination to the locus to be treated, for instance by dissolving, dispersing or diffusing the composition, and/or to facilitate its storage, transport or handling without impairing its effectiveness. They include any and all solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the immunogenic peptide in the composition. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders. Suitable pharmaceutical carriers for use in the pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic add or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives typically contain 8 to 22 carbon atoms. Examples of alkylarylsulphonates are the sodium, calcium or alcanolamine salts of dodecyl benzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidyl-ethanolamine, phosphatidylserine, phosphatidylglycerine, lysolecithin, cardiolipin, dioctanylphosphatidylcholine, dipalmitoylphoshatidylcholine and their mixtures.

Suitable non-ionic surfactants include polyethoxylated and poly propoxylated derivatives of alkyl phenols, fatty alcohols, fatty acids, aliphatic amines or amides containing at least 12 carbon atoms in the molecule, alkylarene sulphonates and dialkylsulphosuccinates, such as polyglycol ether derivatives of aliphatic and cycloaliphatic alcohols, saturated and unsaturated fatty acids and alkylphenols, the derivatives typically containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediamino-polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenaxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants. Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C₈C₂₂ alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbucw’, 2 d ed. (Hanser Verlag, Vienna, 1981) and “Encyclopedia of Surfactants, (Chemical Publishing Co., New York, 1981). Peptides, homologues or derivatives thereof according to the invention (and their physiologically acceptable salts or pharmaceutical compositions all included in the term “active ingredients”) may be administered by any route appropriate to the condition to be treated and appropriate for the compounds, here the proteins and fragments to be administered. Possible routes include regional, systemic, oral (solid form or inhalation), rectal, nasal, topical (including ocular, buccal and sublingual), vaginal and parenteral (including subcutaneous, intramuscular, intravenous, intradermal, intra-arterial, intrathecal and epidural). The preferred route of administration may vary with for example the condition of the recipient or with the diseases to be treated. As described herein, the carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intraarterial, intrathecal and epidural) administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Typical unit dosage formulations are those containing a daily close or unit daily sub-dose, as herein above recited, or an appropriate fraction thereof, of an active ingredient. It should be understood that in addition to the ingredients particularly mentioned above the formulations of this invention may include other agents conventional in the art having regard to the type of formulation in question, for example those suitable for oral administration may include flavouring agents. Peptides, homologues or derivatives thereof according to the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, microspheres, microemulsions, nanoparticles, nanocapsules and so on. Depending on the route of administration, the pharmaceutical composition may require protective coatings. Pharmaceutical forms suitable for injection include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof. In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments.

In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

Cytolytic CD4+ T cells as obtained in the present invention, induce APC apoptosis after MHC-class II dependent cognate activation, affecting both dendritic and B cells, as demonstrated in vitro and in vivo, and (2) suppress bystander T cells by a contact-dependent mechanism in the absence of IL-10 and/or TGF-beta. Cytolytic CD4+ T cells can be distinguished from both natural and adaptive Tregs, as discussed in detail in WO2008/017517.

The immunogenic peptides of the invention containing hydrophobic residues that confer the capacity to bind to the CD1d molecule. Upon administration, are taken up by APC, directed to the late endosome where they are loaded onto CD1d and presented at the surface of the APC. Once presented by CD1d molecule, the oxidoreductase motif in the peptides enhances the capacity to activate NKT cells, becoming cytolytic NKT cells. Said immunogenic peptides activate the production of cytokine, such as IFN-gamma, which will activate other effector cells including CD4+ T cells andnCD8+ T cells. Both CD4+ and CD8+ T cells can participate in the elimination of the cell presenting the antigen as discussed in detail in WO2012/069568.

The present invention will now be illustrated by means of the following examples which are provided without any limiting intention. Furthermore, all references described herein are explicitly included herein by reference.

### EXAMPLES

### Example 1: Design of Immunogenic Peptides with an Oxidoreductase Motif Comprising a Different Number of Amino Acids in Between the 2 Cysteines (C-X_{N}-C)

Different variants of IMCY-0443 or IMCY-0189, two immunogenic peptides comprising a classical oxidoreductase motif (H)CPYC (SEQ ID NO 34 and 35), a linker GW, a murine Myelin Oligodendrocyte Glycoprotein (MOG) MHCII T cell epitope YRSPFSRW (SEQ ID NO: 36) and a flanking sequence HLYR (SEQ ID NO: 122), were designed and synthesized in a way that the sequences were modified to have a different number of amino acids between the 2 cysteines of the oxidoreductase motif (Table 1). Basic amino acids (K or R) were added at the N-terminus or within the oxidoreductase motif. Control peptides with alanine instead of cysteine residues were also synthesized (so-called AA controls). IMCY-0017 (wt MOG35-55 peptide), IMCY-0069 (peptide comprising a classical oxidoreductase motif HCPYC (SEQ ID NO: 35) and a modified murine pre-proinsulin epitope) and IMCY-0257 (peptide comprising a classical oxidoreductase motif HCPYC (SEQ ID NO: 35) and a DBY antigen epitope) were also designed as controls (table 1)

TABLE 1
immunogenic peptides with an oxidoreductase motif
comprising different number of amino acids in
between the 2 cysteines (C-XN-C).

| | | | SEQ |
|---|---|---|---|
| IMCY-No | SEQUENCE | Purpose | ID # |
| IMCY-0443 | CPYC-GWYRSPFSRVVHLYR-NH₂ | Control | 37 |
| IMCY-0189 | HCPYC-GWYRSPFSRVVHLYR-NH₂ | Control | 38 |
| IMCY-0190 | HAPYA-GWYRSPFSRVVHLYR-NH₂ | Control | 39 |
| IMCY-0451 | KCC-GWYRSPFSRVVHLYR-NH₂ | Test | 40 |
| IMCY-0511 | KAA-GWYRSPFSRVVHLYR-NH₂ | Test | 41 |
| IMCY-0452 | CC-GWYRSPFSRVVHLYR-NH₂ | Test | 42 |
| IMCY-0453 | KCRC-GWYRSPFSRVVHLYR-NH₂ | Test | 43 |
| IMCY-0513 | KARA-GWYRSPFSRVVHLYR-NH₂ | Test | 44 |
| IMCY-0454 | CRC-GWYRSPFSRVVHLYR-NH₂ | Test | 45 |
| IMCY-0512 | ARA-GWYRSPFSRVVHLYR-NH₂ | Test | 46 |
| IMCY-0455 | KCRPYC-GWYRSPFSRVVHLYR-NH₂ | Test | 47 |
| IMCY-0515 | KARPYA-GWYRSPFSRVVHLYR-NH₂ | Test | 48 |
| IMCY-0456 | CRPYC-GWYRSPFSRVVHLYR-NH₂ | Test | 49 |
| IMCY-0514 | ARPYA-GWYRSPFSRVVHLYR-NH₂ | Test | 50 |
| IMCY-0017 | MEVGWYRSPFSRVVHLYRNGK-NH₂ | Control | 51 |
| IMCY-0257 | HCPYC-NGGFNSNRANSSRSS-NH₂ | Control | 52 |
| IMCY-0069 | HCPYC-LALWEPKPTQAFVK-NH₂ | Control | 53 |

Example 2: Assessment of the Oxidoreductase Activity of the Immunogenic Peptides

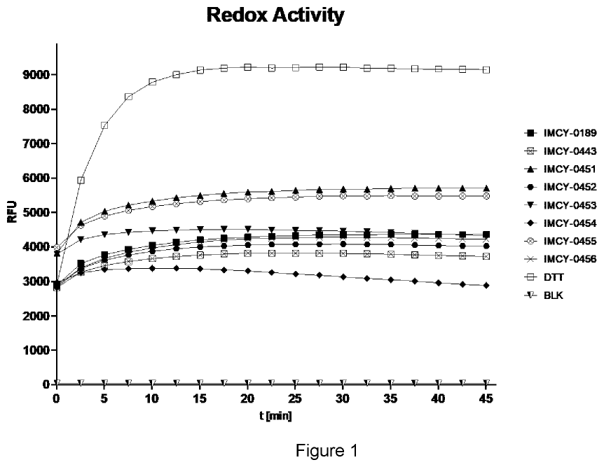

The oxidoreductase activity of the immunogenic peptides is determined using a fluorescent assay described in Tomazzolli et al. (2006) Anal. Biochem. 350, 105-112. Two peptides with a F1TC label become self-quenching when they form a covalent disulfide bond. Upon reduction by a peptide in accordance with the present invention, the reduced individual F1TC labelled peptides emit fluorescence again. The activity is expressed as the mean of duplicates. The results are expressed in Relative Fluorescent Units (RFU). FIG. 1 shows the oxidoreductase activity of the peptide's variants. As expected, the AA controls do not show any oxidoreductase activity (not shown). Control peptides IMCY-0017, -0257 and -0069 were not tested. All the peptides tested displayed an oxidoreductase activity, either similar to or lower than the activity of IMCY-0189, or higher for the KCC or KCRPYC (SEQ ID NO: 54) motifs.

### Example 3: Effect of the Prophylactic Administration of Immunogenic Peptides with an Oxidoreductase Motif Comprising Different Number of Amino Acids in Between the 2 Cysteine (C—X_{N}—C) on Experimental Auto-Immune Encephalomyelitis (EAE) Development in Mice

### Groups of Mice and Dosing

In the study, the C57BL/6 female mice (Taconic Biosiences, 10 weeks old on Day 0) were used. Mice were acclimated for 10 days prior to the first injection (Day −21). They were assigned to 14 groups (12 mice per group) according to Table 2. The distribution of the mice was performed in a balanced manner to achieve similar average weight across the groups at the start of the study (Day −21). Peptides were administered subcutaneously (s.c.), once each day according to Table 2. Each mouse received an injection at two sites (0.05 mL/site), for a total of 0.1 mL/mouse/dosing day, corresponding to 30 μg of peptide per mouse.

### Peptide Preparation

Immunogenic peptides comprising an oxidoreductase motif (cf. table 2), a linker GW, a murine Myelin Oligodendrocyte Glycoprotein (MOG) MHCII T cell epitope YRSPFSRW (SEQ ID NO: 36) and a flanking sequence HLYR (SEQ ID NO: 122), were designed and synthesized as in Example 1.

The following procedure was performed before each immunization. Lyophilized peptides were thawed from −20° C. at room temperature for 10 minutes. Then, they were solubilized in Sodium Acetate buffer (50 mM, pH 5.4) 0.9% NaCL at 1 mg/mL, incubated at room temperature for 5 minutes and vortexed 3 sec. The peptide was diluted in Sodium Acetate buffer (50 mM, pH 5.4) 0.9% NaCL at 0.6 mg/mL. The peptide was mixed with Imject™ Alum adjuvant and vortexed for 5 seconds before injection. At each injection, each mouse received 50 μl adjuvant (2 mg aluminium hydroxide/2 mg magnesium hydroxide) and 50 μl solubilized peptide (30 μg).

The negative control mice were treated with Imject™ Alum mixed with Sodium Acetate buffer (50 mM, pH 5.4) 0.9% NaCL (at each injection, per mouse, 50 μl adjuvant and 50 μl buffer).

TABLE 2
Treatment regimen

| | | | | Dosing | | |
|---|---|---|---|---|---|---|
| | # | Treatment | | Days | Treatment | |
| Group | animals | (s.c) | Motif | Treatment | dose | Purpose |
| 1 | 12 | Alum | N/A | −21, −14, −7 | / | Negative |
| | | | | | | control |
| 2 | 12 | IMCY-0443/Alum | CPYC- | −21, −14, −7 | 30 μg | Test |
| | | | (SEQ ID | | | |
| | | | NO: 34) | | | |
| 3 | 12 | IMCY-0189/Alum | HCPYC- | −21, −14, −7 | 30 μg | Test |
| | | | (SEQ ID | | | |
| | | | NO: 35) | | | |
| 4 | 12 | IMCY-0455/Alum | KCRPYC- | −21, −14, −7 | 30 μg | Test |
| | | | (SEQ ID | | | |
| | | | NO: 54) | | | |
| 5 | 12 | IMCY-0451/Alum | KCC- | −21, −14, −7 | 30 μg | Test |
| 6 | 12 | IMCY-0454/Alum | CRC- | −21, −14, −7 | 30 μg | Test |
| 7 | 12 | IMCY-0453/Alum | KCRC- | −21, −14, −7 | 30 μg | Test |
| | | | (SEQ ID | | | |
| | | | NO: 11) | | | |
| 8 | 12 | IMCY-0456/Alum | CRPYC- | −21, −14, −7 | 30 μg | Test |
| | | | (SEQ ID | | | |
| | | | NO: 55) | | | |
| 9 | 12 | IMCY-0190/Alum | HAPYA- | −21, −14, −7 | 30 μg | Test |
| | | | (SEQ ID | | | |
| | | | NO: 132) | | | |
| 10 | 12 | IMCY-0515/Alum | KARPYA- | −21, −14, −7 | 30 μg | Test |
| | | | (SEQ ID | | | |
| | | | NO: 133) | | | |
| 11 | 12 | IMCY-0511/Alum | KAA- | −21, −14, −7 | 30 μg | Test |
| 12 | 12 | IMCY-0512/Alum | ARA- | −21, −14, −7 | 30 μg | Test |
| 13 | 12 | IMCY-0513/Alum | KARA- | −21, −14, −7 | 30 μg | Test |
| | | | (SEQ ID | | | |
| | | | NO: 120) | | | |
| 14 | 12 | IMCY-0514/Alum | ARPYA- | −21, −14, −7 | 30 μg | Test |
| | | | (SEQ ID | | | |
| | | | NO: 121) | | | |

EAE Induction in Mice

EAE was induced at Day 0 using the following procedure:
Day 0, Hour 0—Immunization with a peptide comprising a T-cell epitope from the mouse Myelin Oligodiendrocyte Glycoprotein (MOG₃₅₋₅₅) specifically binding the MHC-II I-Ab receptor of C57BL/6 mouse.

Mice were injected subcutaneously with the emulsion component of the Hooke Kit™ MOG₃₅₋₅₅/CFA Emulsion PTX (Cat. #EK-2110, Lot #127, Hooke Laboratories, Lawrence Mass.). The peptide MOG_{35,55} (0.2 mg/mouse) was administered in emulsion with CFA (1 mg MOG₃₅₋₅₅/mL emulsion and 2.5 mg killed Mycobacterium tuberculosis H37Ra/ml-emulsion). The emulsion was administered at two different sites in the back. The first injection site, the area of the upper back, was approximately 1 cm caudal of the neckline. The second site, the area of the lower back, was approximately 2 cm cranial of the base of the tail. The injection volume was 0.1 ml at each site.
Day 0, Hour 2—Injection of pertussis toxin

0.1 mL of the pertussis toxin, the component of the same kit, was administered intraperitoneally within 2 hours of the injection of emulsion. The pertussis toxin (lot #1008, Hooke Laboratories) was administered at 100 ng/dose.

Readouts

The EAE scores were used as readout. From Day 7 until the end of the study (Day 28), the animals were scored daily. The person who performed the scoring was unaware of both treatment and of the previous score of each mouse (blinded scoring). EAE was scored on the scale 0 to 5, as shown in Table 3. In-between scores were assigned when the clinical signs fell between the two below defined scores.

TABLE 3
EAE scoring criteria

| Score | Clinical observations |
|---|---|
| 0 | No obvious changes in motor functions of the mouse. |
| | When picked up by the tail, the tail has tension and is erect. |
| | Hind legs are usually spread apart. When the mouse is walking, |
| | no gait or head tilting can be observed. |
| 1 | Limp tail. |
| | When the mouse is picked up by the tail, instead of being erect, |
| | the whole tail drapes over the finger of the operator. |
| 2 | Limp tail and weakness of hind legs. |
| | When the mouse is picked up by the tail, legs are not spread apart |
| | but held closely together. Furthermore, it displays clearly apparent |
| | wobbly walk. |
| 3 | Limp tail and complete paralysis of hind legs (most common), |
| | OR |
| | Limp tail with paralysis of one front and one hind leg, |
| | OR |
| | All the following: |
| | Severe head tilting, |
| | Walking only along the edges of the cage, |
| | Pushing against the cage wall, |
| | Spinning when picked up by the tail. |
| 4 | Limp tail, complete paralysis of the hind legs and partial paralysis |
| | of the front legs. |
| | Mouse is minimally moving around the cage but is alert and |
| | feeding. |
| | Euthanasia is recommended if the mouse scores 4 for 2 days in a |
| | row. |
| 5 | After being euthanized, a mouse is scored 5 for the rest of the |
| | experiment. |

Statistical Analysis

The area under the curve (AUC) and Mean Maximal Score (MMS) were analysed by performing Kruskal-Wallis tests (non-parametric one-way ANOVA method). Significant differences are referred as follows: *p<0.05, **p<0.01, ***p0.001, ****p0.0001.

Results

One mouse in IMCY-0189 group was discovered on Day 8 of the study to have malocclusion creating a risk of malnourishment. The mouse was euthanized and eliminated from the study.

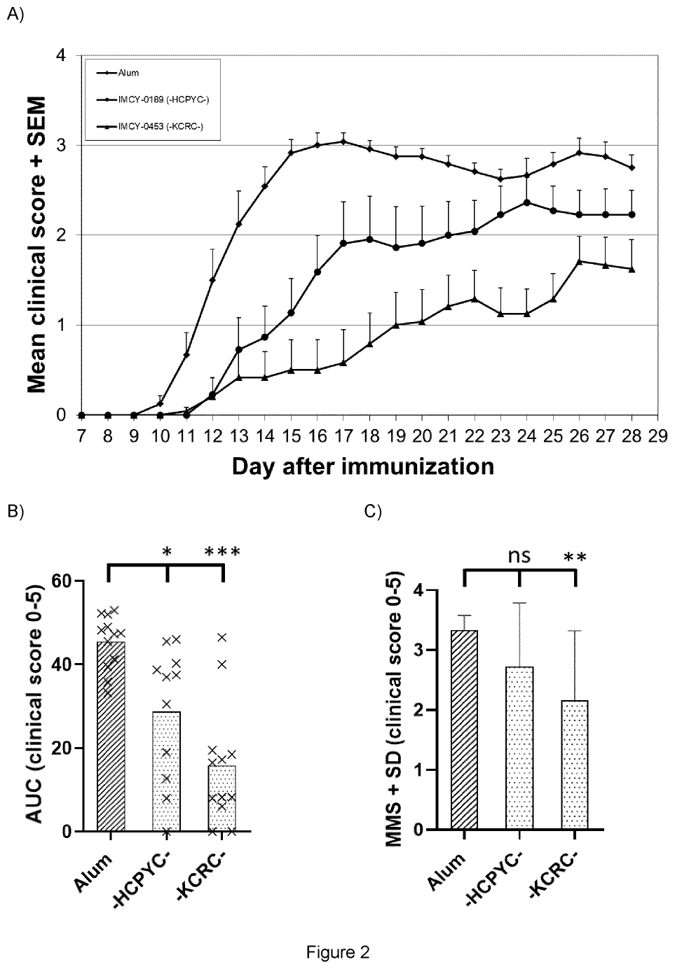

EAE development was evaluated by scoring the mice daily starting from Day 7. To illustrate this evaluation, 3 representative groups are displayed in FIG. 2A. Mice in the Alum group develop the typical EAE for this model. To quantify the efficacy of the treatment, AUC of the clinical score was compared between the Alum group and the two others treated group of mice from Day 7 to Day 28 (FIG. 2B). AUC of the clinical score considered any disease onset delay, as well as the severity of the disease throughout the study. The onset of the disease was delayed in IMCY-0189 and IMCY-0453 treated mice but the severity of the disease was more reduced with IMCY-0453. Moreover, mice treated with IMCY-0453 exhibited a significant reduction of the peak of the disease or MMS (FIG. 2C).

To quantify the efficacy of all peptide treatments, multiple comparisons of the mean rank of each group with the mean rank of the Alum group were performed for AUC and MMS.

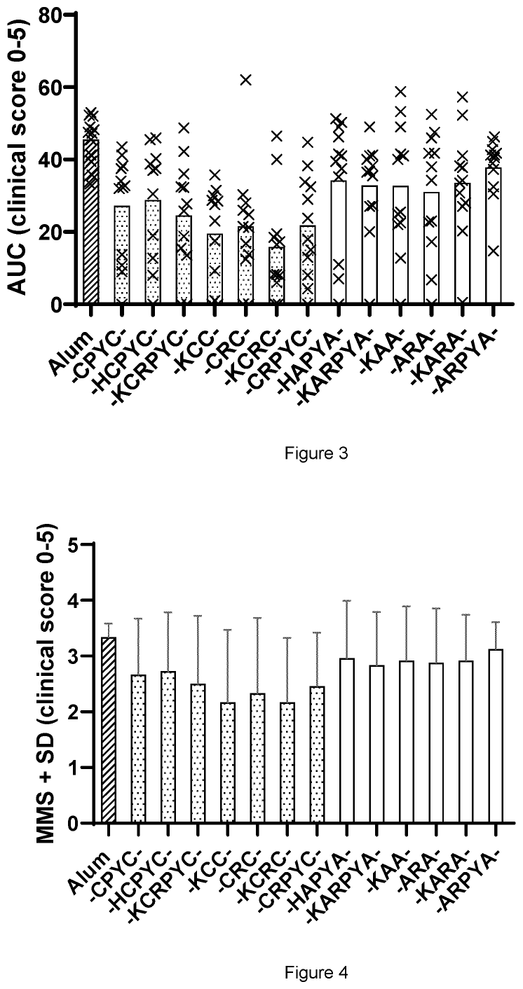

AUC of the clinical score was compared between the Alum group and each treated group of mice from Day 7 to Day 28. As expected, statistical analyses did not show any significant reduction of AUC for the groups treated with AA-peptides (white bars, FIG. 3) in comparison with the control group (hatched bar, FIG. 3). Each of the peptides showed a significant reduction of AUC in comparison with the control group (dotted bars, FIG. 3; Table 4). The non-statistical significance observed with IMCY-189 in this multiple comparison statistical context is due to the low number of mice in this group, which lacks one mice compared to the other groups.

Table 4: Statistical Quantification (p-Values Obtained for Each CC-Peptide in Comparison with the Alum Group) of the AUC Reduction in Mice Treated with Different Peptides.

Peptide Preparation

Immunogenic peptides comprising an oxidoreductase motif (cf. table 4), a linker GW, a murine Myelin Oligodendrocyte Glycoprotein (MOG) MHCII T cell epitope YRSPFSRW (SEQ ID NO: 36) and a flanking sequence HLYR (SEQ ID NO: 122), were designed and synthesized as in Example 1.

TABLE 4

| | | p value |
|---|---|---|
| | | AUC |
| | | reduction |
| | | (Peptide |
| Peptide | Motif | vs. Alum) |
| IMCY-0453 | KCRC- (SEQ ID NO: 11) | **** |
| IMCY-0451 | KCC- | *** |
| IMCY-0454 | CRC- | *** |
| IMCY-0456 | CRPYC- (SEQ ID NO: 55) | ** |
| IMCY-0455 | KCRPYC- (SEQ ID NO: 54) | ** |
| IMCY-0443 | CPYC- (SEQ ID NO: 34) | * |
| IMCY-0189 | HCPYC- (SEQ ID NO: 35) | ns |

MMS of the clinical score was also compared between the Alum group (Alum) and each treated group of mice. As expected, statistical analyses did not show any significant reduction of MMS for the groups treated with AA-peptides (white bars, FIG. 4) in comparison with the control group (hatched bar, FIG. 4). The four CC-peptides with the motifs KCRC (SEQ ID NO: 11), KCC, CRC or CRPYC (SEQ ID NO: 55) showed a significant reduction of MMS in comparison with the control group (dotted bars, FIG. 4; Table 5). A significant reduction of MMS after treatment with both immunogenic peptides comprising a classical oxidoreductase motif (IMCY-0443 or IMCY-0189) was not observed.

Table 5: Statistical Quantification (p-Values Obtained for Each CC-Peptide in Comparison with the Alum Group) of the MMS Reduction in Mice Treated with Different Peptides.

Peptide Preparation

Immunogenic peptides comprising an oxidoreductase motif (cf. table 5), a linker GW, a murine Myelin Oligodendrocyte Glycoprotein (MOG) MHCII T cell epitope YRSPFSRW (SEQ ID NO: 36) and a flanking sequence HLYR (SEQ ID NO: 122), were designed and synthesized as in Example 1.

TABLE 5

| | | p value |
|---|---|---|
| | | MMS |
| | | reduction |
| | | (Peptide |
| Peptide | Motif | vs. Alum) |
| IMCY-0453 | KCRC- (SEQ ID NO: 11) | ** |
| IMCY-0451 | KCC- | * |
| IMCY-0454 | CRC- | * |
| IMCY-0456 | CRPYC- (SEQ ID NO: 55) | * |
| IMCY-0455 | KCRPYC- (SEQ ID NO: 54) | ns |
| IMCY-0443 | CPYC- (SEQ ID NO: 34) | ns |
| IMCY-0189 | HCPYC-(SEQ ID NO: 35) | ns |

### Example 4: Effect of the Therapeutic Administration of Immunogenic Peptides with an Oxidoreductase Motif Comprising Different Number of Amino Acids in Between the 2 Cysteine (C-X_{N}-C) on Experimental Auto-Immune Encephalomyelitis (EAE) Development in Mice

Peptide Preparation

Immunogenic peptides comprising an oxidoreductase motif (cf. table 6), a linker GW, a murine Myelin Oligodendrocyte Glycoprotein (MOG) MHCII T cell epitope YRSPFSRW (SEQ ID NO: 36) and a flanking sequence HLYR (SEQ ID NO: 122), were designed and synthesized as in Example 1.

Groups of Mice and Dosing

The study used a total of 64 female C57BL/6 mice (Taconic Biosciences, 9 weeks old on Day 0). Mice were acclimated for 7 days prior to the first injection. Mice were assigned to groups in a balanced manner to achieve similar average weight across the groups at the start of the study. Table 6 below shows the treatment administered to each group.

TABLE 6
Treatment regimen

| | | | | Dosing | | |
|---|---|---|---|---|---|---|
| | | Treat- | | Days | Treat- | |
| | # | ment | | Treat- | ment | |
| Group | animals | (s.c) | Motif | ment | dose | Purpose |
| 1 | 16 | Saline | N/A | 4, 9, | / | Negative |
| | | | | 14, 19 | | control |
| 2 | 16 | IMCY- | HCPYC- | 4, 9, | 30 μg | Test |
| | | 0189/ | (SEQ | 14, 19 | | |
| | | Alum | ID | | | |
| | | | NO: 35) | | | |
| 3 | 16 | IMCY- | KCRPYC- | 4, 9, | 30 μg | Test |
| | | 0455/ | (SEQ | 14, 19 | | |
| | | Alum | ID | | | |
| | | | NO: 54) | | | |
| 4 | 16 | IMCY- | KCRC- | 4, 9, | 30 μg | Test |
| | | 0453 | (SEQ | 14, 19 | | |
| | | /Alum | ID | | | |
| | | | NO: 11) | | | |

Dosing of all mice was performed once on each of the days indicated in Table 6, s.c., at a volume of 0.05 mL/site, each mouse receiving injection at two sites, for a total of 0.1 mL/mouse/dosing day. IMCY-0189, IMCY-0453 or IMCY-0455 peptide total close was 30 pg per administration.

Compound Preparation

For Saline treatment, 0.9% NaCl solution was prepared at each dosing day. Peptide's preparation was performed as described in example 3.

Serum Neurofilaments Levels Determination

On Day 28, blood was collected from all mice into gel dot activator tubes and allowed to dot at room temperature for ˜30 minutes. Blood is then centrifuged at ˜10000 g for 5 minutes. Serum was transferred into Eppendorf tubes and stored at −80° C. until shipment to Quanterix™. Serum Neurofilament light (NF-L) protein levels were quantified using Simoa® NF-light Advantage kit, a digital immunoassay for the quantitative determination of NF-L in serum, plasma and CSF. The used antibodies (Uman Diagnostics, Umeå Sweden) also cross react with murine, bovine and macaque NF-L epitopes and as such, this assay can be used for research with these species. All samples were tested in duplicate at a dilution factor of 40×.

Terminal Collection

At the end of the study, all mice were euthanized, and spines were collected and placed in 10% buffered formalin for histological analysis.

Histology

For each spine, one H&E stained slide and one anti-MBP stained slide were prepared and analyzed. Each slide contained a section with samples from lumbar, thoracic and cervical of spinal cord (3 samples). All analysis was performed by a pathologist blinded to the experimental groups and all clinical readouts.

Inflammatory foci of approximately 20 cells were counted in each H&E stained section. When inflammatory infiltrates consisted of more than 20 cells, an estimate was made of how many foci of 20 cells were present.

Demyelination was scored in each anti-MBP (using immunohistochemistry) stained section. In anti-MBP sections, demyelination is observed as conspicuous unstained areas in white matter tracts and is associated with presence of large vacuoles. The demyelination score represents an estimate of demyelinated area for each section as follows:
0—no demyelination (less than 5% demyelinated area)
1—5 to 20% demyelinated area
2—20 to 40% demyelinated area
3—40 to 60% demyelinated area
4—60 to 80% demyelinated area
5—80 to 100% demyelinated area

Statistical Analysis

AUC, MMS, inflammation and demyelination, and NF-L levels quantification data were analyzed by performing Ordinary one-way ANOVA. Adjustment for multiplicity was performed using Holm-Sidak's method. Significant differences are referred as follows: *p<0.05, **p<0.01, ***p<0.001, ****p<0.0001.

EAE induction and scoring were performed as described in example 3.

Results and Interpretation of Data

EAE Scoring

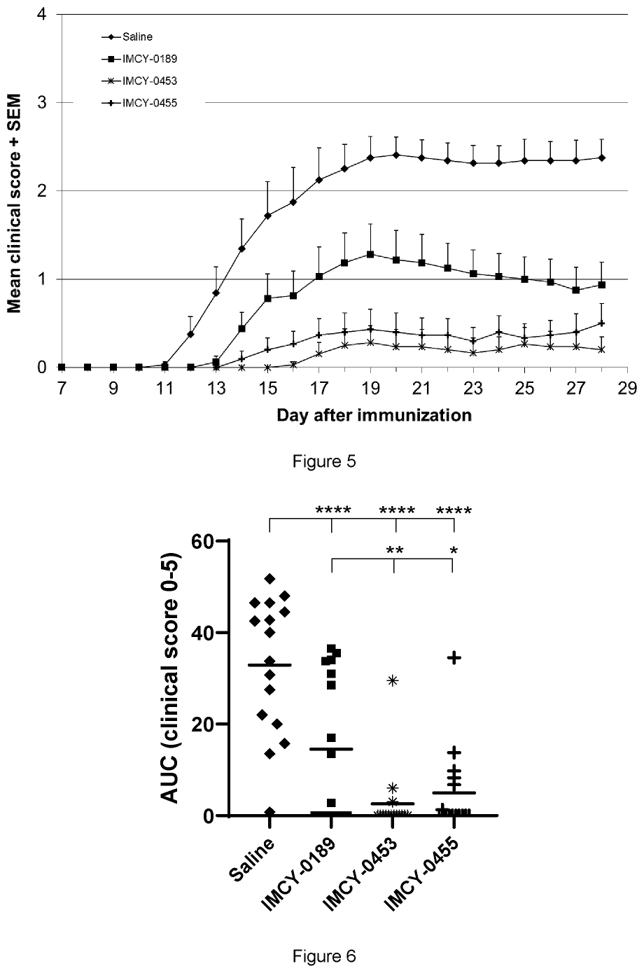

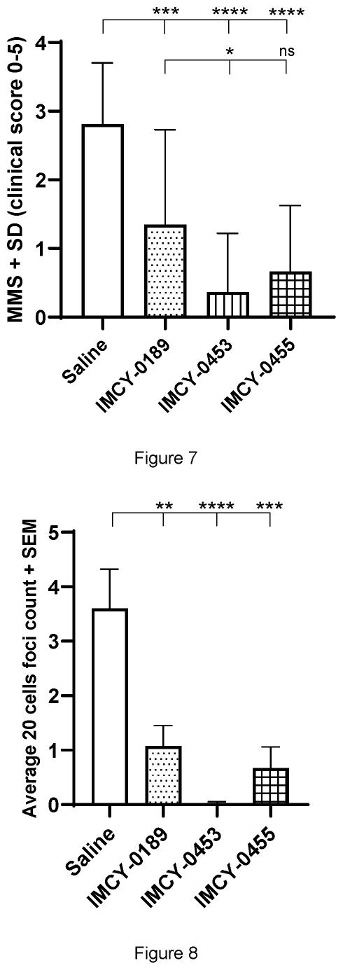

EAE development was evaluated by comparing clinical EAE readouts for all groups to the negative control (Saline) group. EAE scoring, AUC (area under the curve) and MMS (mean maximal score) are presented in FIGS. 5, 6 and 7.

Mice of the Saline group (negative control) developed somewhat milder EAE but still within the expected range for this model. No mice died in this group.

Mice treated with IMCY-0189 showed postponed disease onset and reduced end score, and statistically reduced AUC and MMS compared to the negative control group. No mice died in this group.

All clinical readouts (disease onset, end score, AUC and MMS) of mice treated with IMCY-0453 or IMCY-0455 were statistically significantly improved as compared to the negative control group, and also, particularly for IMCY-0453, as compared to IMCY-0189. One (1) mouse of the IMCY-453 group and one (1) mouse of the IMCY-455 group died, but the death of these mice did not appear to be due to EAE, and therefore they were excluded from analysis.

Histology

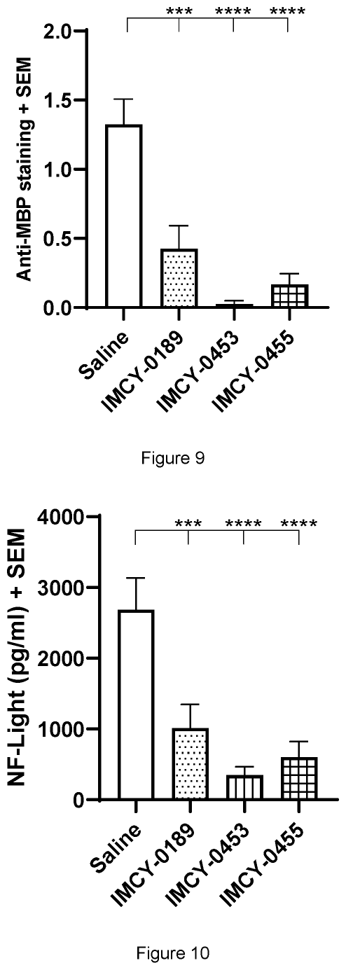

Histological readouts were evaluated by comparing inflammation and demyelination levels for all groups to the negative control (Saline) group. Inflammation and demyelination data are presented in FIGS. 8 and 9.

Histological results for the Saline group (negative control) were consistent with the clinical findings and as expected for this model.

Mice treated with IMCY-0189, IMCY-0453 and IMCY-0455 showed statistically significant reduced level of both inflammation and demyelination as compared to the control group, and particularly for IMCY-0453 with which inflammation and demyelination are nearly abolished.

Serum Neurofilament Levels

Axonal damage was also evaluated by comparing NF-L levels for all groups to the negative control (Saline) group. Data are presented in FIG. 10.

NF-L levels for the Saline group (negative control) were consistent with the clinical findings and as expected for this model.

Mice treated with the IMCY-0189, IMCY-0453 and IMCY-0455 showed statistically significant reduced NF-L levels as compared to the negative control group.

### Example 5: Ability of the Immunogenic Peptides with an Oxidoreductase Motif Comprising a Different Number of Amino Acids in Between the 2 Cysteines (C—X_{N}-C) to Induce Specific CD4+ T Cells with Lytic Properties

2D2 transgenic mice (Jackson Laboratory) were used as a source of CD4+ cell. These mice were shown to contain Myelin Oligodendrocyte Glycoprotein (MOG) specific, self-reactive T cell repertoire, which makes them an appropriate candidate to study the reactivity of homogenous population of CD4+ to the current experimental peptides. In this study, 2D2-Total CD4+ cells were purified using manufacture instruction (Miltenyi Biotec, 130-104-454) and antigen presenting cells were splenocyte depleted for T cell repertoire (CD90.2 depletion, Miltenyi Biotec, 130-104-454) from C57BL6 mice (compatible with 2D2-transgenic mice). Control conditions for every study were either no-peptide addition or addition of IMCY-0017, IMCY-0257 or IMCY-0069 (see example 1 and table 1) which the two later, can bind to MHCII from C57BL6-APC efficiently but not activate the TCR from 2D2-transgenic CD4+ T cells. AA controls were not used.

Comparison of the ability of the different variants to engage Ag-specific CD4+ cells First, it was investigated whether modification of the sequence (variants of IMCY-0189) would affect the global capacity to activate monoclonal Ag-specific T cells, by measuring the activation marker CD154+(CD40L). To achieve this goal, co-cultures of 2D2-Total CD4+ and C57BL6 APCs (Ratio 1:1) were exposed to the final concentration of 5 μM of each experimental and control peptide in the presence of CD40-blocking antibodies, for 16 h. After incubation, the frequency of Ag-specific cells was determined (Pro-19-004-v00) for all experimental and control conditions by flow cytometry (FIG. 11). These results suggested that all the modified variants of IMCY-0189 (IMCY-0451 to IMCY-0456) have been able to induce the engagement of the higher percentage of Ag-specific cell (CD4+CD154+) compared with IMCY-0189 (FIG. 11; dotted line).

Comparison of the Ability of the Different Variants to Induce sIL2 in 2D2-CD4+ Cells

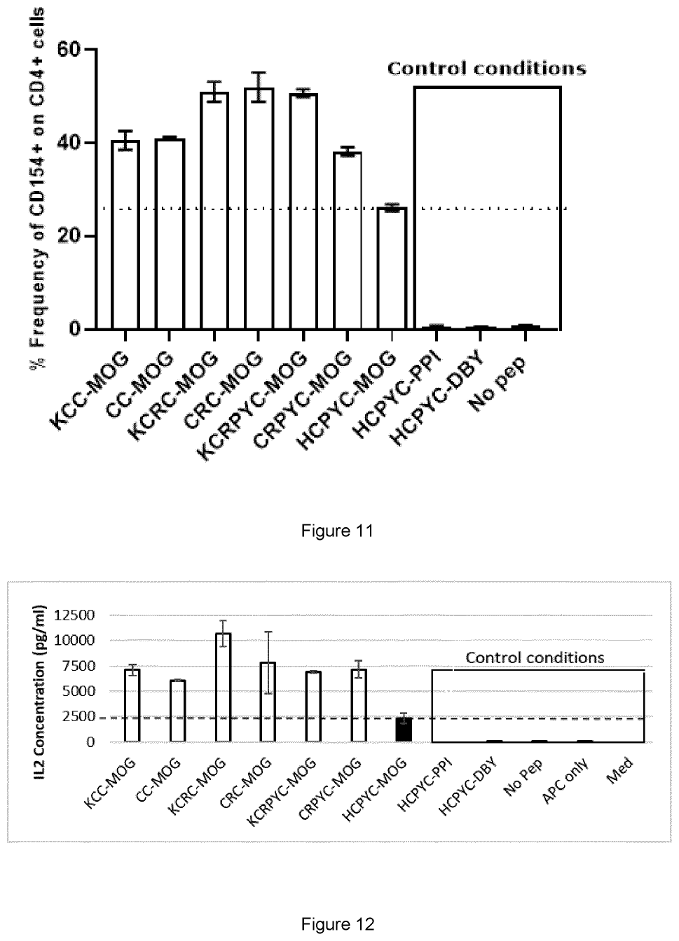

It was next tried to determine if the increased ability of the different variants to engage Ag-specific CD4+ cells would lead to increased induction of IL2 by activated those cells. To this end, co-cultures of 2D2-Total CD4+ and mitomycin C-treated C57BL6 APCs (Ratio 1:1) were exposed to the final concentration of 5 μM of each experimental and control peptide for 24 h. After incubation time, supernatant (SN) was collected by addition of halt protease inhibitor cocktail (Thermo scientific; 78430) and stored at −80° C. until the amount of secreted IL2 (sIL2) for all experimental and control conditions was measured using flow cytometry (LEGENDplex mouse Th cytokine panel (13-plex); Cat No. 740005; Pro-19-007-v00). The FACS results suggested that the modified variants (IMCY-0451 to IMCY-0456) were able to induce a higher amount of sIL2 compared with IMCY-0189 (FIG. 12; dotted line).

Comparison of the Ability of the Different Variants to Induce Lytic Markers on CD4+ T Cells.

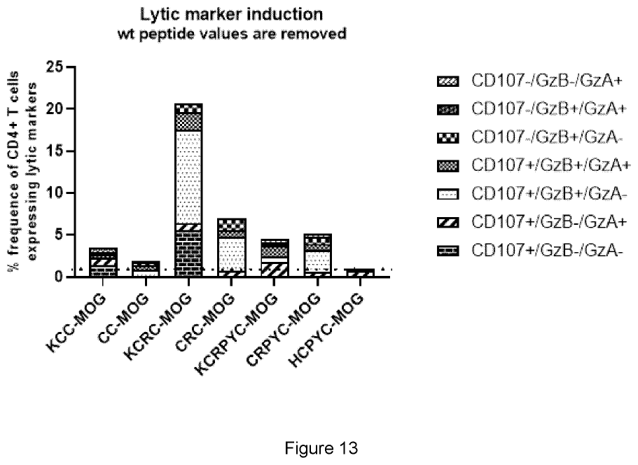

Immunogenic peptides comprising an MCHII T cell epitope and a classical oxidoreductase motif of sequence CxxC are known from the art to induce CD4+ cells with lytic markers called cytolytic CD4+ cells (cCD4+). Therefore, it was attempted to determine whether the peptide variants of the present invention would lead to earlier and/or stronger (percentage or intensity) lytic properties in activated Ag-specific CD4 T cells. In this set of experiments, 2D2-Total CD4+ were stimulated with mitomycin C-treated C57BL6 APCs (Ratio 1:1) and the final concentration of 5 μM of each experimental and control peptide (S1). After an interval of 10-14 days, all cell cultures from all experimental and control conditions were stimulated again (S2D0) in the absence and presence of regarding peptides. 16 h after the second stimulation (S2D1) cells were collected and stained for intracellular (IC) lytic markers measurement (Granzyme A and B, CD107a and b) by flow cytometry (Pro-19-014-v00). Expectedly, the control peptide conditions (No pep, IMCY-0257 and IMCY-0069) did not survive in the cell culture (no expansion in response to addition of no peptide or mismatch TCR peptides) and could not be included in the measurement at S2. The FACS results were then determined for all variants of IMCY-0189 and the measurement for the wild type peptide (IMCY-0017) was removed from all the other measurements to determine purely the effect of every modified oxidoreductase motif on the induction of lytic properties. This result so far suggests that modified variants of IMCY-0189 (IMCY-0451 to IMCY-0456) with a large variability were able to induce a higher percentage of lytic markers compared with IMCY-0189 (FIG. 13; dotted line).

1. An immunogenic peptide having oxidoreductase activity, said immunogenic peptide comprising:
a) an oxidoreductase motif;
b) a T-cell epitope of an antigenic protein; and
c) a linker between a) and b) of between 0 and 7 amino acids, preferably of between 0 and 4 amino acids;
wherein said oxidoreductase motif a) has the following general structure: Zₘ-C-Xₙ-C-,
wherein Z is any amino acid, preferably a basic amino acid, more preferably selected from the group comprising: K, H, R and a non-natural basic amino acid, preferably K or H, more preferably K; wherein m is an integer selected from the group comprising: 1, 0, or 2;
wherein X is any amino acid, preferably a basic amino acid, more preferably selected from the group comprising: K, H, R and a non-natural basic amino acid, preferably K or H, more preferably R; wherein n is an integer selected from the group comprising: 1, 0, or 3;
wherein said oxidoreductase motif is not part of a repeat of the standard C-XX-[CST](SEQ ID NO: 2) or [CST]-XX-C(SEQ ID NO: 1) oxidoreductase motifs such as repeats of said motif which can be spaced from each other by one or more amino acids (e.g. CXXC X CXXC X CXXC (SEQ ID NO: 6)), as repeats which are adjacent to each other (CXXCCXXCCXXC (SEQ ID NO: 7)) or as repeats which overlap with each other CXXCXXCXXC (SEQ ID NO: 8) or CXCCXCCXCC (SEQ ID NO: 9)); and
wherein said antigenic protein is an autoantigen involved in type-1 diabetes (T1D), a demyelinating disorder such as multiple sclerosis (MS) or neuromyelitis optica (NMO), or in rheumatoid arthritis (RA).

2. The immunogenic peptide according to claim 1, wherein n is 1 and m is 1 or 0;
wherein X is a basic amino acid, more preferably selected from the group comprising: K, H, R and a non-natural basic amino acid, preferably K or H, more preferably R;
wherein Z, when present, is a basic amino acid, more preferably selected from the group comprising: K, H, R and a non-natural basic amino acid, preferably K or H, more preferably K.

3. The immunogenic peptide according to claim 2, wherein said oxidoreductase motif is C[KRH]C-, such as CRC-, CKC-, CHC-; or
wherein said oxidoreductase motif is -[KHR]C[KRH]C-, such as KCRC-, KCKC-, KCHC-, RCRC-, RCKC-, RCHC-, HCRC-, HCKC-, HCHC-, corresponding to SEQ ID NO: 10 to 19 respectively,
most preferably wherein said oxidoreductase motif is KCRC- (SEQ ID NO: 11).

4. The immunogenic peptide according to claim 1, wherein n is 0 and wherein m is 1, 0, 2 or 3, wherein Z, when present, is a basic amino acid, more preferably selected from the group comprising: K, H, R and a non-natural basic amino acid, preferably K or H, more preferably K.

5. The immunogenic peptide according to claim 4, wherein said oxidoreductase motif is [HKR]CC-, such as HCC-, RCC-, or KCC-.

6. The immunogenic peptide according to claim 1, wherein n is 3 and m is 1 or 0;
wherein at least one X is a basic amino acid, more preferably selected from the group comprising: K, H, R and a non-natural basic amino acid, preferably K or H, more preferably R; wherein Z, when present, is a basic amino acid, more preferably selected from the group comprising: K, H, R and a non-natural basic amino acid, preferably K or H, more preferably K.

7. The immunogenic peptide according to claim 6, wherein said oxidoreductase motif is C[KHR]XXC-, such as CRXXC-, CHXXC-, CKXXC-, corresponding to SEQ ID NO: 20 to 23 respectively, preferably wherein one X in the motif is P or Y; or
wherein said oxidoreductase motif is -[KHR]C[KHR]XXC-, such as KCRXXC-, KCHXXC-, KCKXXC-, HCRXXC-, HCHXXC-, HCKXXC-, RCRXXC-, RCHXXC-, or RCKXXC-, corresponding to SEQ ID NO: 24 to 33 respectively, preferably wherein one X in the motif is P or Y,
most preferably wherein said oxidoreductase motif is KCRPYC- (SEQ ID NO: 54), or CRPYC- (SEQ ID NO: 55).

8. The immunogenic peptide according to any one of claims 1 to 7, wherein said T cell epitope of an antigenic protein is an NKT cell epitope having a length of between 7 and 25 amino acids; or wherein said T cell epitope of an antigenic protein is an MHC class II T cell epitope having a length of between 7 and 25 amino acids.

9. The immunogenic peptide according to any one of claims 1 to 8, wherein said immunogenic peptide has a length of between 9 and 50 amino acids.

10. The immunogenic peptide according to any one of claims 1 to 9, wherein said oxidoreductase motif does not naturally occur in the amino acid sequence of sad antigen within a region of 11 amino acids N-terminally or C-terminally of said T-cell epitope in said antigenic protein.

11. and/or wherein the amino acid sequence of the antigen of said T-cell epitope does not comprise said oxidoreductase motif.

12. The immunogenic peptide according to any one of claims 1 to 11, which is an artificial peptide, not naturally occurring in nature.

13. The immunogenic peptide according to any one of aspects 1 to 12, wherein said immunogenic peptide comprises a T-cell epitope derived from the Myelin-oligodendrocyte glycoprotein (MOG) antigen amino acid sequence selected from YRSPFSRW (SEQ ID NO: 36) and YRPPFSRW (human SEQ ID NO: 123), and comprises as a linker the amino acid sequence GW and comprises as a flanker the amino acid sequence HLYR (SEQ ID NO: 122).

14. The immunogenic peptide according to any one of aspects 1 to 12, wherein said immunogenic peptide comprises a T-cell epitope derived from the Myelin-oligodendrocyte glycoprotein (MOG) antigen amino acid sequence selected from FLRVPCWKI (SEQ ID NO: 124), and FLRVPSWKI (SEQ ID NO: 125), and comprises as a linker the amino acid sequence VRY and comprises as a flanker an amino acid sequence selected from: TLF, TLFK (SEQ ID NO: 126), or TLFKK (SEQ ID NO: 127).

15. The immunogenic peptide according to any one of aspects 1 to 12, wherein said immunogenic peptide is selected from the group consisting of:
(SEQ ID NO: 128)
KHCPYCVRYFLRVPSWKITLFKK,
(SEQ ID NO: 129)
KHCPYCVRYFLRVPCWKITLFKK.
(SEQ ID NO: 130)
HCPYCVRYFLRVPSWKITLF,
(SEQ ID NO: 131)
HCPYCVRYFLRVPCWKITLF,
(SEQ ID NO: 38)
HCPYCGWYRSPFSRVVHLYR,
(SEQ ID NO: 43)
KCRCGWYRSPFSRVVHLYR,
and
(SEQ ID NO: 47)
KCRPYCGWYRSPFSRVVHLYR.

16. A polynucleotide (nucleic acid molecule) encoding the immunogenic peptide according to any one of claims 1 to 15, preferably selected from isolated desoxyribonucleic acid (DNA), plasmid DNA (pDNA), coding DNA (cDNA), ribonucleic acid (RNA), messenger RNA (mRNA) or modified versions thereof.

17. The immunogenic peptide according to any one of claims 1 to 15, or the polynucleotide according to claim 16, for use in treating and/or prevention of an autoimmune disease, an infection with an intracellular pathogen, a tumor, an allograft rejection, or an immune response to a soluble allofactors, to an allergen exposure or to a viral vector used for gene therapy or gene vaccination.

18. The immunogenic peptide according to any one of claims 1 to 15, or the polynucleotide according to claim 16, wherein said antigenic protein is selected from the group consisting of: (pro)insulin, GAD65, GAD67, IA-2 (ICA512), IA-2 (beta/phogrin), IGRP, Chromogranin, ZnT8 and HSP-60, for use in treating and/or prevention of type-1 diabetes (T1D).

19. The immunogenic peptide according to any one of claims 1 to 15, or the polynucleotide according to claim 16, wherein said antigenic protein is selected from the group consisting of: Myelin oligodendrocyte glycoprotein (MOG), Myelin basic protein (MBP), Proteolipid protein (PLP), Myelin-oligodendrocytic basic protein (MOBP), and Oligodendrocyte-specific protein (OSP), for use in treating and/or prevention of multiple sclerosis (MS), and/or neuromyelitis optica (NMO).

20. The immunogenic peptide according to any one of claims 1 to 15, or the polynucleotide according to claim 16, wherein said antigenic protein is selected from the group consisting of: GRP78, HSP60, 60 kDa chaperonin 2, Gelsolin, Chitinase-3-like protein 1, Cathepsin S, Serum albumin, and Cathepsin D, for use in treating and/or prevention of or rheumatoid arthritis (RA).

21. The immunogenic peptide according to any one of claims 1 to 15, or the polynucleotide according to claim 16, wherein said antigenic protein is Myelin oligodendrocyte glycoprotein (MOG, for use in treating and/or prevention of neuromyelitis optica (NMO).

22. A method for obtaining a population of antigen-specific cytolytic CD4+ T cells, against APC presenting said antigen, the method comprising the steps of:
providing peripheral blood cells,
contacting said cells with an immunogenic peptide according to any one of claims 1 to 15 or with the polynucleotide according to claim 16; and
expanding said cells in the presence of IL-2.

23. A method for obtaining a population of antigen-specific NKT cells, the method comprising the steps of:
providing peripheral blood cells,
contacting said cells with an immunogenic peptide according to any one of claims 1 to 15 or with the polynucleotide according to claim 16; and
expanding said cells in the presence of IL-2.

24. The population of antigen-specific cytolytic CD4+ T cells obtainable by the method of claim 22, for use in the treatment and/or prevention of type-1 diabetes (T1D), multiple sclerosis (MS), neuromyelitis optica (NMO), or rheumatoid arthritis (RA).

25. The population of antigen-specific NKT cells obtainable by the method of claim 23, for use in the treatment and/or prevention of type-1 diabetes (T1D), multiple sclerosis (MS), neuromyelitis optica (NMO), or rheumatoid arthritis (RA).

26. A method of treating of, ameliorating the symptoms of, and/or preventing of an autoimmune disease in a subject, comprising the steps of administering the immunogenic peptide according to anyone of claims 1 to 15, the polynucleotide of claim 16, or the cell population according to claim 24 or 25 to said subject.

27. A method of treating of, ameliorating the symptoms of and/or preventing of an autoimmune disease in a subject, comprising the steps of:
providing peripheral blood cells of said subject,
contacting said cells with an antigenic peptide according to any one of claims 1 to 15, or with the polynucleotide of claim 16,
expanding said cells in vitro, and
administering said expanded cells to said individual.

28. The method according to claim 26 or 27, wherein said autoimmune disease is type-1 diabetes (T1D), a demyelinating disorder such as multiple sclerosis (MS) or neuromyelitis optica (NMO), or rheumatoid arthritis (RA).

29. The method according to claim 28, wherein said antigenic protein is selected from the group consisting of: (pro)insulin, GAD65, GAD67, IA-2 (ICA512), IA-2 (beta/phogrin), IGRP, Chromogranin, ZnT8 and HSP-60, and wherein said autoimmune disease is type-1 diabetes (T1D).

30. The method according to claim 28, wherein said antigenic protein is selected from the group consisting of: Myelin oligodendrocyte glycoprotein (MOG), Myelin basic protein (MBP), Proteolipid protein (PLP), Myelin-oligodendrocytic basic protein (MOBP), and Oligodendrocyte-specific protein (OSP), and wherein said autoimmune disease is multiple sclerosis (MS), and/or neuromyelitis optica (NMO).

31. The method according to claim 28, wherein said antigenic protein is selected from the group consisting of: GRP78, HSP60, 60 kDa chaperonin 2, Gelsolin, Chitinase-3-like protein 1, Cathepsin S, Serum albumin, and Cathepsin D, and wherein said autoimmune disease is rheumatoid arthritis (RA).

32. The method according to claim 28, wherein said antigenic protein is Myelin oligodendrocyte glycoprotein (MOG), and wherein said autoimmune disease is neuromyelitis optica (NMO).


SEQUENCE LISTING (678 sequences)

SEQ ID NO: 1 - protein, 4 aa, Artificial Sequence
  synthetic peptide
  misc_feature (2)..(3): Xaa can be any naturally occurring amino acid
  PEPTIDE (4)..(4): C, S, or T
CXXX

SEQ ID NO: 2 - protein, 4 aa, Artificial Sequence
  synthetic peptide
  PEPTIDE (1)..(1): C, S, or T
  misc_feature (2)..(3): Xaa can be any naturally occurring amino acid
XXXC

SEQ ID NO: 3 - protein, 9 aa, Homo sapiens
LALEGSLQK

SEQ ID NO: 4 - protein, 9 aa, Homo sapiens
FLRVPCWKI

SEQ ID NO: 5 - protein, 9 aa, Artificial Sequence
  synthetic peptide
FLRVPSWKI

SEQ ID NO: 6 - protein, 14 aa, Artificial Sequence
  synthetic peptide
  misc_feature (2)..(3): Xaa can be any naturally occurring amino acid
  misc_feature (5)..(5): Xaa can be any naturally occurring amino acid
  misc_feature (7)..(8): Xaa can be any naturally occurring amino acid
  misc_feature (10)..(10): Xaa can be any naturally occurring amino acid
  misc_feature (12)..(13): Xaa can be any naturally occurring amino acid
CXXCXCXXCXCXXC

SEQ ID NO: 7 - protein, 12 aa, Artificial Sequence
  synthetic peptide
  misc_feature (2)..(3): Xaa can be any naturally occurring amino acid
  misc_feature (6)..(7): Xaa can be any naturally occurring amino acid
  misc_feature (10)..(11): Xaa can be any naturally occurring amino acid
CXXCCXXCCXXC

SEQ ID NO: 8 - protein, 10 aa, Artificial Sequence
  synthetic peptide
  misc_feature (2)..(3): Xaa can be any naturally occurring amino acid
  misc_feature (5)..(6): Xaa can be any naturally occurring amino acid
  misc_feature (8)..(9): Xaa can be any naturally occurring amino acid
CXXCXXCXXC

SEQ ID NO: 9 - protein, 10 aa, Artificial Sequence
  synthetic peptide
  misc_feature (2)..(2): Xaa can be any naturally occurring amino acid
  misc_feature (5)..(5): Xaa can be any naturally occurring amino acid
  misc_feature (8)..(8): Xaa can be any naturally occurring amino acid
CXCCXCCXCC

SEQ ID NO: 10 - protein, 4 aa, Artificial Sequence
  synthetic peptide
  PEPTIDE (1)..(1): K, H, or R
  PEPTIDE (3)..(3): K, H, or R
XCXC

SEQ ID NO: 11 - protein, 4 aa, Artificial Sequence
  synthetic peptide
KCRC

SEQ ID NO: 12 - protein, 4 aa, Artificial Sequence
  synthetic peptide
KCKC

SEQ ID NO: 13 - protein, 4 aa, Artificial Sequence
  synthetic peptide
KCHC

SEQ ID NO: 14 - protein, 4 aa, Artificial Sequence
  synthetic peptide
RCRC

SEQ ID NO: 15 - protein, 4 aa, Artificial Sequence
  synthetic peptide
RCKC

SEQ ID NO: 16 - protein, 4 aa, Artificial Sequence
  synthetic peptide
RCHC

SEQ ID NO: 17 - protein, 4 aa, Artificial Sequence
  synthetic peptide
HCRC

SEQ ID NO: 18 - protein, 4 aa, Artificial Sequence
  synthetic peptide
HCKC

SEQ ID NO: 19 - protein, 4 aa, Artificial Sequence
  synthetic peptide
HCHC

SEQ ID NO: 20 - protein, 5 aa, Artificial Sequence
  synthetic peptide
  PEPTIDE (2)..(2): K, H, or R
  misc_feature (3)..(4): Xaa can be any naturally occurring amino acid
CXXXC

SEQ ID NO: 21 - protein, 5 aa, Artificial Sequence
  synthetic peptide
  misc_feature (3)..(4): Xaa can be any naturally occurring amino acid
CRXXC

SEQ ID NO: 22 - protein, 5 aa, Artificial Sequence
  synthetic peptide
  misc_feature (3)..(4): Xaa can be any naturally occurring amino acid
CHXXC

SEQ ID NO: 23 - protein, 5 aa, Artificial Sequence
  synthetic peptide
  misc_feature (3)..(4): Xaa can be any naturally occurring amino acid
CKXXC

SEQ ID NO: 24 - protein, 6 aa, Artificial Sequence
  synthetic peptide
  PEPTIDE (1)..(1): K, H, or R
  PEPTIDE (3)..(3): K, H, or R
  misc_feature (4)..(5): Xaa can be any naturally occurring amino acid
XCXXXC

SEQ ID NO: 25 - protein, 6 aa, Artificial Sequence
  synthetic peptide
  misc_feature (4)..(5): Xaa can be any naturally occurring amino acid
KCRXXC

SEQ ID NO: 26 - protein, 6 aa, Artificial Sequence
  synthetic peptide
  misc_feature (4)..(5): Xaa can be any naturally occurring amino acid
KCHXXC

SEQ ID NO: 27 - protein, 6 aa, Artificial Sequence
  synthetic peptide
  misc_feature (4)..(5): Xaa can be any naturally occurring amino acid
KCKXXC

SEQ ID NO: 28 - protein, 6 aa, Artificial Sequence
  synthetic peptide
  misc_feature (4)..(5): Xaa can be any naturally occurring amino acid
HCRXXC

SEQ ID NO: 29 - protein, 6 aa, Artificial Sequence
  synthetic peptide
  misc_feature (4)..(5): Xaa can be any naturally occurring amino acid
HCHXXC

SEQ ID NO: 30 - protein, 6 aa, Artificial Sequence
  synthetic peptide
  misc_feature (4)..(5): Xaa can be any naturally occurring amino acid
HCKXXC

SEQ ID NO: 31 - protein, 6 aa, Artificial Sequence
  synthetic peptide
  misc_feature (4)..(5): Xaa can be any naturally occurring amino acid
RCRXXC

SEQ ID NO: 32 - protein, 6 aa, Artificial Sequence
  synthetic peptide
  misc_feature (4)..(5): Xaa can be any naturally occurring amino acid
RCHXXC

SEQ ID NO: 33 - protein, 6 aa, Artificial Sequence
  synthtetic peptide
  misc_feature (4)..(5): Xaa can be any naturally occurring amino acid
RCKXXC

SEQ ID NO: 34 - protein, 4 aa, Artificial Sequence
  synthetic peptide
CPYC

SEQ ID NO: 35 - protein, 5 aa, Artificial Sequence
  synthetic peptide
HCPYC

SEQ ID NO: 36 - protein, 9 aa, Artificial Sequence
  synthetic peptide
YRSPFSRVV

SEQ ID NO: 37 - protein, 19 aa, Artificial Sequence
  synthetic peptide
CPYCGWYRSPFSRVVHLYR

SEQ ID NO: 38 - protein, 20 aa, Artificial Sequence
  synthetic peptide
HCPYCGWYRSPFSRVVHLYR

SEQ ID NO: 39 - protein, 20 aa, Artificial Sequence
  synthetic peptide
HAPYAGWYRSPFSRVVHLYR

SEQ ID NO: 40 - protein, 18 aa, Artificial Sequence
  synthetic peptide
KCCGWYRSPFSRVVHLYR

SEQ ID NO: 41 - protein, 18 aa, Artificial Sequence
  synthetic peptide
KAAGWYRSPFSRVVHLYR

SEQ ID NO: 42 - protein, 17 aa, Artificial Sequence
  synthetic peptide
CCGWYRSPFSRVVHLYR

SEQ ID NO: 43 - protein, 19 aa, Artificial Sequence
  synthetic peptide
KCRCGWYRSPFSRVVHLYR

SEQ ID NO: 44 - protein, 19 aa, Artificial Sequence
  synthetic peptide
KARAGWYRSPFSRVVHLYR

SEQ ID NO: 45 - protein, 18 aa, Artificial Sequence
  synthetic peptide
CRCGWYRSPFSRVVHLYR

SEQ ID NO: 46 - protein, 18 aa, Artificial Sequence
  synthetic peptide
ARAGWYRSPFSRVVHLYR

SEQ ID NO: 47 - protein, 21 aa, Artificial Sequence
  synthetic peptide
KCRPYCGWYRSPFSRVVHLYR

SEQ ID NO: 48 - protein, 21 aa, Artificial Sequence
  synthetic peptide
KARPYAGWYRSPFSRVVHLYR

SEQ ID NO: 49 - protein, 20 aa, Artificial Sequence
  synthetic peptide
CRPYCGWYRSPFSRVVHLYR

SEQ ID NO: 50 - protein, 20 aa, Artificial Sequence
  synthetic peptide
ARPYAGWYRSPFSRVVHLYR

SEQ ID NO: 51 - protein, 21 aa, Artificial Sequence
  synthetic peptide
MEVGWYRSPFSRVVHLYRNGK

SEQ ID NO: 52 - protein, 20 aa, Artificial Sequence
  synthetic peptide
HCPYCNGGFNSNRANSSRSS

SEQ ID NO: 53 - protein, 19 aa, Artificial Sequence
  synthetic peptide
HCPYCLALWEPKPTQAFVK

SEQ ID NO: 54 - protein, 6 aa, Artificial Sequence
  synthetic peptide
KCRPYC

SEQ ID NO: 55 - protein, 5 aa, Artificial Sequence
  synthetic peptide
CRPYC

SEQ ID NO: 56 - protein, 4 aa, Artificial Sequence
  synthetic peptide
  PEPTIDE (1)..(1): C, S, or T
  misc_feature (2)..(3): Xaa can be any naturally occurring amino acid
XXXC

SEQ ID NO: 57 - protein, 5 aa, Artificial Sequence
  synthetic peptide
  PEPTIDE (1)..(1): C, S, or T
  misc_feature (2)..(4): Xaa can be any naturally occurring amino acid
XXXXC

SEQ ID NO: 58 - protein, 6 aa, Artificial Sequence
  synthetic peptide
  PEPTIDE (1)..(1): C, S, or T
  misc_feature (2)..(5): Xaa can be any naturally occurring amino acid
XXXXXC

SEQ ID NO: 59 - protein, 7 aa, Artificial Sequence
  synthetic peptide
  PEPTIDE (1)..(1): C, S, or T
  misc_feature (2)..(6): Xaa can be any naturally occurring amino acid
XXXXXXC

SEQ ID NO: 60 - protein, 8 aa, Artificial Sequence
  synthetic peptide
  PEPTIDE (1)..(1): C, S, or T
  misc_feature (2)..(7): Xaa can be any naturally occurring amino acid
XXXXXXXC

SEQ ID NO: 61 - protein, 4 aa, Artificial Sequence
  synthetic peptide
  misc_feature (1)..(1): Xaa can be any naturally occurring amino acid
  PEPTIDE (2)..(2): C, S, or T
  misc_feature (3)..(3): Xaa can be any naturally occurring amino acid
XXXC

SEQ ID NO: 62 - protein, 5 aa, Artificial Sequence
  synthetic peptide
  misc_feature (1)..(1): Xaa can be any naturally occurring amino acid
  PEPTIDE (2)..(2): C, S, or T
  misc_feature (3)..(4): Xaa can be any naturally occurring amino acid
XXXXC

SEQ ID NO: 63 - protein, 6 aa, Artificial Sequence
  synthetic peptide
  misc_feature (1)..(1): Xaa can be any naturally occurring amino acid
  PEPTIDE (2)..(2): C, S, or T
  misc_feature (3)..(5): Xaa can be any naturally occurring amino acid
XXXXXC

SEQ ID NO: 64 - protein, 7 aa, Artificial Sequence
  synthetic peptide
  misc_feature (1)..(1): Xaa can be any naturally occurring amino acid
  PEPTIDE (2)..(2): C, S, or T
  misc_feature (3)..(6): Xaa can be any naturally occurring amino acid
XXXXXXC

SEQ ID NO: 65 - protein, 8 aa, Artificial Sequence
  synthetic peptide
  misc_feature (1)..(1): Xaa can be any naturally occurring amino acid
  PEPTIDE (2)..(2): C, S, or T
  misc_feature (3)..(7): Xaa can be any naturally occurring amino acid
XXXXXXXC

SEQ ID NO: 66 - protein, 9 aa, Artificial Sequence
  synthetic peptide
  misc_feature (1)..(1): Xaa can be any naturally occurring amino acid
  PEPTIDE (2)..(2): C, S, or T
  misc_feature (3)..(8): Xaa can be any naturally occurring amino acid
XXXXXXXXC

SEQ ID NO: 67 - protein, 4 aa, Artificial Sequence
  synthetic peptide
  misc_feature (1)..(2): Xaa can be any naturally occurring amino acid
  PEPTIDE (3)..(3): C, S, or T
XXXC

SEQ ID NO: 68 - protein, 5 aa, Artificial Sequence
  synthetic peptide
  misc_feature (1)..(2): Xaa can be any naturally occurring amino acid
  PEPTIDE (3)..(3): C, S, or T
  misc_feature (4)..(4): Xaa can be any naturally occurring amino acid
XXXXC

SEQ ID NO: 69 - protein, 6 aa, Artificial Sequence
  synthetic peptide
  misc_feature (1)..(2): Xaa can be any naturally occurring amino acid
  PEPTIDE (3)..(3): C, S, or T
  misc_feature (4)..(5): Xaa can be any naturally occurring amino acid
XXXXXC

SEQ ID NO: 70 - protein, 7 aa, Artificial Sequence
  synthetic peptide
  misc_feature (1)..(2): Xaa can be any naturally occurring amino acid
  PEPTIDE (3)..(3): C, S, or T
  misc_feature (4)..(6): Xaa can be any naturally occurring amino acid
XXXXXXC

SEQ ID NO: 71 - protein, 8 aa, Artificial Sequence
  synthetic peptide
  misc_feature (1)..(2): Xaa can be any naturally occurring amino acid
  PEPTIDE (3)..(3): C, S, or T
  misc_feature (4)..(7): Xaa can be any naturally occurring amino acid
XXXXXXXC

SEQ ID NO: 72 - protein, 9 aa, Artificial Sequence
  synthetic peptide
  misc_feature (1)..(2): Xaa can be any naturally occurring amino acid
  PEPTIDE (3)..(3): C, S, or T
  misc_feature (4)..(8): Xaa can be any naturally occurring amino acid
XXXXXXXXC

SEQ ID NO: 73 - protein, 10 aa, Artificial Sequence
  synthetic peptide
  misc_feature (1)..(2): Xaa can be any naturally occurring amino acid
  PEPTIDE (3)..(3): C, S, or T
  misc_feature (4)..(9): Xaa can be any naturally occurring amino acid
XXXXXXXXXC

SEQ ID NO: 74 - protein, 5 aa, Artificial Sequence
  synthetic peptide
  misc_feature (1)..(3): Xaa can be any naturally occurring amino acid
  PEPTIDE (4)..(4): C, S, or T
XXXXC

SEQ ID NO: 75 - protein, 6 aa, Artificial Sequence
  synthetic peptide
  misc_feature (1)..(5): Xaa can be any naturally occurring amino acid
XXXXXC

SEQ ID NO: 76 - protein, 7 aa, Artificial Sequence
  synthetic peptide
  misc_feature (1)..(3): Xaa can be any naturally occurring amino acid
  PEPTIDE (4)..(4): C, S, or T
  misc_feature (5)..(6): Xaa can be any naturally occurring amino acid
XXXXXXC

SEQ ID NO: 77 - protein, 8 aa, Artificial Sequence
  synthetic peptide
  misc_feature (1)..(3): Xaa can be any naturally occurring amino acid
  PEPTIDE (4)..(4): C, S, or T
  misc_feature (5)..(7): Xaa can be any naturally occurring amino acid
XXXXXXXC

SEQ ID NO: 78 - protein, 9 aa, Artificial Sequence
  synthetic peptide
  misc_feature (1)..(3): Xaa can be any naturally occurring amino acid
  PEPTIDE (4)..(4): C, S, or T
  misc_feature (5)..(8): Xaa can be any naturally occurring amino acid
XXXXXXXXC

SEQ ID NO: 79 - protein, 10 aa, Artificial Sequence
  synthetic peptide
  misc_feature (1)..(3): Xaa can be any naturally occurring amino acid
  PEPTIDE (4)..(4): C, S, or T
  misc_feature (5)..(9): Xaa can be any naturally occurring amino acid
XXXXXXXXXC

SEQ ID NO: 80 - protein, 11 aa, Artificial Sequence
  synthetic peptide
  misc_feature (1)..(3): Xaa can be any naturally occurring amino acid
  PEPTIDE (4)..(4): C, S, or T
  misc_feature (5)..(10): Xaa can be any naturally occurring amino acid
XXXXXXXXXXC

SEQ ID NO: 81 - protein, 4 aa, Artificial Sequence
  synthetic peptide
  misc_feature (2)..(3): Xaa can be any naturally occurring amino acid
  PEPTIDE (4)..(4): C, S, or T
CXXX

SEQ ID NO: 82 - protein, 5 aa, Artificial Sequence
  synthetic peptide
  misc_feature (2)..(4): Xaa can be any naturally occurring amino acid
  PEPTIDE (5)..(5): C, S, or T
CXXXX

SEQ ID NO: 83 - protein, 6 aa, Artificial Sequence
  synthetic peptide
  misc_feature (2)..(5): Xaa can be any naturally occurring amino acid
  PEPTIDE (6)..(6): C, S, or T
CXXXXX

SEQ ID NO: 84 - protein, 7 aa, Artificial Sequence
  synthetic peptide
  misc_feature (2)..(6): Xaa can be any naturally occurring amino acid
  PEPTIDE (7)..(7): C, S, or T
CXXXXXX

SEQ ID NO: 85 - protein, 8 aa, Artificial Sequence
  synthetic peptide
  misc_feature (2)..(7): Xaa can be any naturally occurring amino acid
  PEPTIDE (8)..(8): C, S, or T
CXXXXXXX

SEQ ID NO: 86 - protein, 4 aa, Artificial Sequence
  synthetic peptide
  misc_feature (1)..(1): Xaa can be any naturally occurring amino acid
  misc_feature (3)..(3): Xaa can be any naturally occurring amino acid
  PEPTIDE (4)..(4): C, S, or T
XCXX

SEQ ID NO: 87 - protein, 5 aa, Artificial Sequence
  synthetic peptide
  misc_feature (1)..(1): Xaa can be any naturally occurring amino acid
  misc_feature (3)..(4): Xaa can be any naturally occurring amino acid
  PEPTIDE (5)..(5): C, S, or T
XCXXX

SEQ ID NO: 88 - protein, 6 aa, Artificial Sequence
  synthetic peptide
  misc_feature (1)..(1): Xaa can be any naturally occurring amino acid
  misc_feature (3)..(5): Xaa can be any naturally occurring amino acid
  PEPTIDE (6)..(6): C, S, or T
XCXXXX

SEQ ID NO: 89 - protein, 7 aa, Artificial Sequence
  synthetic peptide
  misc_feature (1)..(1): Xaa can be any naturally occurring amino acid
  misc_feature (3)..(6): Xaa can be any naturally occurring amino acid
  PEPTIDE (7)..(7): C, S, or T
XCXXXXX

SEQ ID NO: 90 - protein, 8 aa, Artificial Sequence
  synthetic peptide
  misc_feature (1)..(1): Xaa can be any naturally occurring amino acid
  misc_feature (3)..(7): Xaa can be any naturally occurring amino acid
  PEPTIDE (8)..(8): C, S, or T
XCXXXXXX

SEQ ID NO: 91 - protein, 9 aa, Artificial Sequence
  synthetic peptide
  misc_feature (1)..(1): Xaa can be any naturally occurring amino acid
  misc_feature (3)..(8): Xaa can be any naturally occurring amino acid
  PEPTIDE (9)..(9): C, S, or T
XCXXXXXXX

SEQ ID NO: 92 - protein, 4 aa, Artificial Sequence
  synthetic peptide
  misc_feature (1)..(2): Xaa can be any naturally occurring amino acid
  PEPTIDE (4)..(4): C, S, or T
XXCX

SEQ ID NO: 93 - protein, 5 aa, Artificial Sequence
  synthetic peptide
  misc_feature (1)..(2): Xaa can be any naturally occurring amino acid
  misc_feature (4)..(4): Xaa can be any naturally occurring amino acid
  PEPTIDE (5)..(5): C, S, or T
XXCXX

SEQ ID NO: 94 - protein, 6 aa, Artificial Sequence
  synthetic peptide
  misc_feature (1)..(2): Xaa can be any naturally occurring amino acid
  misc_feature (4)..(5): Xaa can be any naturally occurring amino acid
  PEPTIDE (6)..(6): C, S, or T
XXCXXX

SEQ ID NO: 95 - protein, 7 aa, Artificial Sequence
  synthetic peptide
  misc_feature (1)..(2): Xaa can be any naturally occurring amino acid
  misc_feature (4)..(6): Xaa can be any naturally occurring amino acid
  PEPTIDE (7)..(7): C, S, or T
XXCXXXX

SEQ ID NO: 96 - protein, 8 aa, Artificial Sequence
  synthetic peptide
  misc_feature (1)..(2): Xaa can be any naturally occurring amino acid
  misc_feature (4)..(7): Xaa can be any naturally occurring amino acid
  PEPTIDE (8)..(8): C, S, or T
XXCXXXXX

SEQ ID NO: 97 - protein, 9 aa, Artificial Sequence
  synthetic peptide
  misc_feature (1)..(2): Xaa can be any naturally occurring amino acid
  misc_feature (4)..(8): Xaa can be any naturally occurring amino acid
  PEPTIDE (9)..(9): C, S, or T
XXCXXXXXX

SEQ ID NO: 98 - protein, 10 aa, Artificial Sequence
  synthetic peptide
  misc_feature (1)..(2): Xaa can be any naturally occurring amino acid
  misc_feature (4)..(9): Xaa can be any naturally occurring amino acid
  PEPTIDE (10)..(10): XXCXXXXXXX
XXCXXXXXXX

SEQ ID NO: 99 - protein, 5 aa, Artificial Sequence
  synthetic peptide
  misc_feature (1)..(3): Xaa can be any naturally occurring amino acid
  PEPTIDE (5)..(5): C, S, or T
XXXCX

SEQ ID NO: 100 - protein, 6 aa, Artificial Sequence
  synthetic peptide
  misc_feature (1)..(3): Xaa can be any naturally occurring amino acid
  misc_feature (5)..(5): Xaa can be any naturally occurring amino acid
  PEPTIDE (6)..(6): C, S, or T
XXXCXX

SEQ ID NO: 101 - protein, 7 aa, Artificial Sequence
  synthetic peptide
  misc_feature (1)..(3): Xaa can be any naturally occurring amino acid
  misc_feature (5)..(6): Xaa can be any naturally occurring amino acid
  PEPTIDE (7)..(7): C, S, or T
XXXCXXX

SEQ ID NO: 102 - protein, 8 aa, Artificial Sequence
  synthetic peptide
  misc_feature (1)..(3): Xaa can be any naturally occurring amino acid
  misc_feature (5)..(7): Xaa can be any naturally occurring amino acid
  PEPTIDE (8)..(8): C, S, or T
XXXCXXXX

SEQ ID NO: 103 - protein, 9 aa, Artificial Sequence
  synthetic peptide
  misc_feature (1)..(3): Xaa can be any naturally occurring amino acid
  misc_feature (5)..(8): Xaa can be any naturally occurring amino acid
  PEPTIDE (9)..(9): C, S, or T
XXXCXXXXX

SEQ ID NO: 104 - protein, 10 aa, Artificial Sequence
  synthetic peptide
  misc_feature (1)..(3): Xaa can be any naturally occurring amino acid
  misc_feature (5)..(9): Xaa can be any naturally occurring amino acid
  PEPTIDE (10)..(10): C, S, or T
XXXCXXXXXX

SEQ ID NO: 105 - protein, 11 aa, Artificial Sequence
  synthetic peptide
  misc_feature (1)..(3): Xaa can be any naturally occurring amino acid
  misc_feature (5)..(10): Xaa can be any naturally occurring amino acid
  PEPTIDE (11)..(11): C, S, or T
XXXCXXXXXXX

SEQ ID NO: 106 - protein, 5 aa, Artificial Sequence
  synthetic peptide
  misc_feature (2)..(4): Xaa can be any naturally occurring amino acid
CXXXC

SEQ ID NO: 107 - protein, 4 aa, Artificial Sequence
  synthetic peptide
  misc_feature (1)..(1): Xaa can be any naturally occurring amino acid
  misc_feature (3)..(3): Xaa can be any naturally occurring amino acid
XCXC

SEQ ID NO: 108 - protein, 6 aa, Artificial Sequence
  synthetic peptide
  misc_feature (1)..(1): Xaa can be any naturally occurring amino acid
  misc_feature (3)..(5): Xaa can be any naturally occurring amino acid
XCXXXC

SEQ ID NO: 109 - protein, 4 aa, Artificial Sequence
  synthetic peptide
  misc_feature (1)..(2): Xaa can be any naturally occurring amino acid
XXCC

SEQ ID NO: 110 - protein, 5 aa, Artificial Sequence
  synthetic peptide
  misc_feature (1)..(2): Xaa can be any naturally occurring amino acid
  misc_feature (4)..(4): Xaa can be any naturally occurring amino acid
XXCXC

SEQ ID NO: 111 - protein, 7 aa, Artificial Sequence
  synthetic peptide
  misc_feature (1)..(2): Xaa can be any naturally occurring amino acid
  misc_feature (4)..(6): Xaa can be any naturally occurring amino acid
XXCXXXC

SEQ ID NO: 112 - protein, 7 aa, Artificial Sequence
  synthetic peptide
  PEPTIDE (1)..(1): F, W, H, or Y
  misc_feature (2)..(3): Xaa can be any naturally occurring amino acid
  PEPTIDE (4)..(4): I, L, M, or V
  misc_feature (5)..(6): Xaa can be any naturally occurring amino acid
  PEPTIDE (7)..(7): F, W, T, H, Y
XXXXXXX

SEQ ID NO: 113 - protein, 7 aa, Artificial Sequence
  synthetic peptide
  PEPTIDE (1)..(1): F, W
  misc_feature (2)..(3): Xaa can be any naturally occurring amino acid
  PEPTIDE (4)..(4): I, L, M, or V
  misc_feature (5)..(6): Xaa can be any naturally occurring amino acid
  PEPTIDE (7)..(7): F, W
XXXXXXX

SEQ ID NO: 114 - protein, 6 aa, Artificial Sequence
  synthetic peptide
  PEPTIDE (1)..(1): D or E
  misc_feature (2)..(4): Xaa can be any naturally occurring amino acid
  PEPTIDE (6)..(6): L or I
XXXXLX

SEQ ID NO: 115 - protein, 5 aa, Artificial Sequence
  synthetic peptide
  misc_feature (2)..(3): Xaa can be any naturally occurring amino acid
DXXLL

SEQ ID NO: 116 - protein, 6 aa, Artificial Sequence
  synthetic peptide
  misc_feature (2)..(4): Xaa can be any naturally occurring amino acid
DXXXLL

SEQ ID NO: 117 - protein, 4 aa, Artificial Sequence
  synthetic peptide
  misc_feature (2)..(3): Xaa can be any naturally occurring amino acid
  PEPTIDE (4)..(4): F, Y, or W
YXXX

SEQ ID NO: 118 - protein, 5 aa, Artificial Sequence
  synthetic peptide
  misc_feature (3)..(4): Xaa can be any naturally occurring amino acid
  PEPTIDE (5)..(5): C, S, or T
HCXXX

SEQ ID NO: 119 - protein, 5 aa, Artificial Sequence
  synthetic peptide
  PEPTIDE (2)..(2): C, S, or T
  misc_feature (3)..(4): Xaa can be any naturally occurring amino acid
HXXXC

SEQ ID NO: 120 - protein, 4 aa, Artificial Sequence
  synthetic peptide
KARA

SEQ ID NO: 121 - protein, 5 aa, Artificial Sequence
  synthetic peptide
ARPYA

SEQ ID NO: 122 - protein, 4 aa, Artificial Sequence
  synthetic peptide
HLYR

SEQ ID NO: 123 - protein, 9 aa, Artificial Sequence
  synthetic peptide
YRPPFSRVV

SEQ ID NO: 124 - protein, 9 aa, Artificial Sequence
  synthetic peptide
FLRVPCWKI

SEQ ID NO: 125 - protein, 9 aa, Artificial Sequence
  synthetic peptide
FLRVPSWKI

SEQ ID NO: 126 - protein, 4 aa, Artificial Sequence
  synthetic peptide
TLFK

SEQ ID NO: 127 - protein, 5 aa, Artificial Sequence
  synthetic peptide
TLFKK

SEQ ID NO: 128 - protein, 23 aa, Artificial Sequence
  synthetic peptide
KHCPYCVRYFLRVPSWKITLFKK

SEQ ID NO: 129 - protein, 23 aa, Artificial Sequence
  synthetic peptide
KHCPYCVRYFLRVPCWKITLFKK

SEQ ID NO: 130 - protein, 20 aa, Artificial Sequence
  synthetic peptide
HCPYCVRYFLRVPSWKITLF

SEQ ID NO: 131 - protein, 20 aa, Artificial Sequence
  synthetic peptide
HCPYCVRYFLRVPCWKITLF

SEQ ID NO: 132 - protein, 5 aa, Artificial Sequence
  synthetic peptide
HAPYA

SEQ ID NO: 133 - protein, 6 aa, Artificial Sequence
  synthetic peptide
KARPYA

SEQ ID NO: 134 - protein, 7 aa, Artificial Sequence
  synthetic peptide
  PEPTIDE (1)..(1): F, W, H, Y, or T
  misc_feature (2)..(3): Xaa can be any naturally occurring amino acid
  PEPTIDE (4)..(4): I, L, M, or V
  misc_feature (5)..(6): Xaa can be any naturally occurring amino acid
  PEPTIDE (7)..(7): F, W, T, H, Y
XXXXXXX

SEQ ID NO: 135 - protein, 7 aa, Artificial Sequence
  synthetic peptide
  PEPTIDE (1)..(1): F, W, H, Y
  misc_feature (2)..(3): Xaa can be any naturally occurring amino acid
  PEPTIDE (4)..(4): I, L, M, or V
  misc_feature (5)..(6): Xaa can be any naturally occurring amino acid
  PEPTIDE (7)..(7): F, W, H, Y
XXXXXXX

SEQ ID NO: 136 - protein, 7 aa, Artificial Sequence
  synthetic peptide
  PEPTIDE (1)..(1): F, W, Y, T
  misc_feature (2)..(3): Xaa can be any naturally occurring amino acid
  PEPTIDE (4)..(4): I, L, M, or V
  misc_feature (5)..(6): Xaa can be any naturally occurring amino acid
  PEPTIDE (7)..(7): F, W, Y, or T
XXXXXXX

SEQ ID NO: 137 - protein, 7 aa, Artificial Sequence
  synthetic peptide
  PEPTIDE (1)..(1): F, W, or Y
  misc_feature (2)..(3): Xaa can be any naturally occurring amino acid
  PEPTIDE (4)..(4): I, L, M, or V
  misc_feature (5)..(6): Xaa can be any naturally occurring amino acid
  PEPTIDE (7)..(7): F, W, or Y
XXXXXXX

SEQ ID NO: 138 - protein, 7 aa, Artificial Sequence
  synthetic peptide
  PEPTIDE (1)..(1): F, W, Y, or H
  misc_feature (2)..(3): Xaa can be any naturally occurring amino acid
  PEPTIDE (4)..(4): I, L, M
  misc_feature (5)..(6): Xaa can be any naturally occurring amino acid
  PEPTIDE (7)..(7): F, W, Y or H
XXXXXXX

SEQ ID NO: 139 - protein, 7 aa, Artificial Sequence
  synthetic peptide
  PEPTIDE (1)..(1): F, W, Y, H, or T
  misc_feature (2)..(3): Xaa can be any naturally occurring amino acid
  PEPTIDE (4)..(4): I, L, M
  misc_feature (5)..(6): Xaa can be any naturally occurring amino acid
  PEPTIDE (7)..(7): F, W, Y, H, or T
XXXXXXX

SEQ ID NO: 140 - protein, 7 aa, Artificial Sequence
  synthetic peptide
  PEPTIDE (1)..(1): F, W, or Y
  misc_feature (2)..(3): Xaa can be any naturally occurring amino acid
  PEPTIDE (4)..(4): I, L, M
  misc_feature (5)..(6): Xaa can be any naturally occurring amino acid
  PEPTIDE (7)..(7): F, W, or Y
XXXXXXX

SEQ ID NO: 141 - protein, 4 aa, Artificial Sequence
  synthetic peptide
KCGC

SEQ ID NO: 142 - protein, 4 aa, Artificial Sequence
  synthetic peptide
KCAC

SEQ ID NO: 143 - protein, 4 aa, Artificial Sequence
  synthetic peptide
KCVC

SEQ ID NO: 144 - protein, 4 aa, Artificial Sequence
  synthetic peptide
KCLC

SEQ ID NO: 145 - protein, 4 aa, Artificial Sequence
  synthetic peptide
KCIC

SEQ ID NO: 146 - protein, 4 aa, Artificial Sequence
  synthetic peptide
KCMC

SEQ ID NO: 147 - protein, 4 aa, Artificial Sequence
  synthetic peptide
KCFC

SEQ ID NO: 148 - protein, 4 aa, Artificial Sequence
  synthetic peptide
KCWC

SEQ ID NO: 149 - protein, 4 aa, Artificial Sequence
  synthetic peptide
KCPC

SEQ ID NO: 150 - protein, 4 aa, Artificial Sequence
  synthetic peptide
KCSC

SEQ ID NO: 151 - protein, 4 aa, Artificial Sequence
  synthetic peptide
KCTC

SEQ ID NO: 152 - protein, 4 aa, Artificial Sequence
  synthetic peptide
KCYC

SEQ ID NO: 153 - protein, 4 aa, Artificial Sequence
  synthetic peptide
KCNC

SEQ ID NO: 154 - protein, 4 aa, Artificial Sequence
  synthetic peptide
KCQC

SEQ ID NO: 155 - protein, 4 aa, Artificial Sequence
  synthetic peptide
KCDC

SEQ ID NO: 156 - protein, 4 aa, Artificial Sequence
  synthetic peptide
KCEC

SEQ ID NO: 157 - protein, 4 aa, Artificial Sequence
  synthetic peptide
HCGC

SEQ ID NO: 158 - protein, 4 aa, Artificial Sequence
  synthetic peptide
HCAC

SEQ ID NO: 159 - protein, 4 aa, Artificial Sequence
  synthetic peptide
HCVC

SEQ ID NO: 160 - protein, 4 aa, Artificial Sequence
  synthetic peptide
HCLC

SEQ ID NO: 161 - protein, 4 aa, Artificial Sequence
  synthetic peptide
HCIC

SEQ ID NO: 162 - protein, 4 aa, Artificial Sequence
  synthetic peptide
HCMC

SEQ ID NO: 163 - protein, 4 aa, Artificial Sequence
  synthetic peptide
HCFC

SEQ ID NO: 164 - protein, 4 aa, Artificial Sequence
  synthetic peptide
HCWC

SEQ ID NO: 165 - protein, 4 aa, Artificial Sequence
  synthetic peptide
HCPC

SEQ ID NO: 166 - protein, 4 aa, Artificial Sequence
  synthetic peptide
HCSC

SEQ ID NO: 167 - protein, 4 aa, Artificial Sequence
  synthetic peptide
HCTC

SEQ ID NO: 168 - protein, 4 aa, Artificial Sequence
  synthetic peptide
HCYC

SEQ ID NO: 169 - protein, 4 aa, Artificial Sequence
  synthetic peptide
HCNC

SEQ ID NO: 170 - protein, 4 aa, Artificial Sequence
  synthetic peptide
HCQC

SEQ ID NO: 171 - protein, 4 aa, Artificial Sequence
  synthetic peptide
HCDC

SEQ ID NO: 172 - protein, 4 aa, Artificial Sequence
  synthetic peptide
HCEC

SEQ ID NO: 173 - protein, 4 aa, Artificial Sequence
  synthetic peptide
RCGC

SEQ ID NO: 174 - protein, 4 aa, Artificial Sequence
  synthetic peptide
RCAC

SEQ ID NO: 175 - protein, 4 aa, Artificial Sequence
  synthetic peptide
RCVC

SEQ ID NO: 176 - protein, 4 aa, Artificial Sequence
  synthetic peptide
RCLC

SEQ ID NO: 177 - protein, 4 aa, Artificial Sequence
  synthetic peptide
RCIC

SEQ ID NO: 178 - protein, 4 aa, Artificial Sequence
  synthetic peptide
RCMC

SEQ ID NO: 179 - protein, 4 aa, Artificial Sequence
  synthetic peptide
RCFC

SEQ ID NO: 180 - protein, 4 aa, Artificial Sequence
  synthetic peptide
RCWC

SEQ ID NO: 181 - protein, 4 aa, Artificial Sequence
  synthetic peptide
RCPC

SEQ ID NO: 182 - protein, 4 aa, Artificial Sequence
  synthetic peptide
RCSC

SEQ ID NO: 183 - protein, 4 aa, Artificial Sequence
  synthetic peptide
RCTC

SEQ ID NO: 184 - protein, 4 aa, Artificial Sequence
  synthetic peptide
RCYC

SEQ ID NO: 185 - protein, 4 aa, Artificial Sequence
  synthetic peptide
RCNC

SEQ ID NO: 186 - protein, 4 aa, Artificial Sequence
  synthetic peptide
RCQC

SEQ ID NO: 187 - protein, 4 aa, Artificial Sequence
  synthetic peptide
RCDC

SEQ ID NO: 188 - protein, 4 aa, Artificial Sequence
  synthetic peptide
RCEC

SEQ ID NO: 189 - protein, 5 aa, Artificial Sequence
  synthetic peptide
  misc_feature (2)..(2): Xaa can be any naturally occurring amino acid
CXPYC

SEQ ID NO: 190 - protein, 5 aa, Artificial Sequence
  synthetic peptide
  misc_feature (3)..(3): Xaa can be any naturally occurring amino acid
CPXYC

SEQ ID NO: 191 - protein, 5 aa, Artificial Sequence
  synthetic peptide
  misc_feature (4)..(4): Xaa can be any naturally occurring amino acid
CPYXC

SEQ ID NO: 192 - protein, 5 aa, Artificial Sequence
  synthetic peptide
CKPYC

SEQ ID NO: 193 - protein, 5 aa, Artificial Sequence
  synthetic peptide
CHPYC

SEQ ID NO: 194 - protein, 5 aa, Artificial Sequence
  synthetic peptide
CGPYC

SEQ ID NO: 195 - protein, 5 aa, Artificial Sequence
  synthetic peptide
CAPYC

SEQ ID NO: 196 - protein, 5 aa, Artificial Sequence
  synthetic peptide
CVPYC

SEQ ID NO: 197 - protein, 5 aa, Artificial Sequence
  synthetic peptide
CLPYC

SEQ ID NO: 198 - protein, 5 aa, Artificial Sequence
  synthetic peptide
CIPYC

SEQ ID NO: 199 - protein, 5 aa, Artificial Sequence
  synthetic peptide
CMPYC

SEQ ID NO: 200 - protein, 5 aa, Artificial Sequence
  synthetic peptide
CFPYC

SEQ ID NO: 201 - protein, 5 aa, Artificial Sequence
  synthetic peptide
CWPYC

SEQ ID NO: 202 - protein, 5 aa, Artificial Sequence
  synthetic peptide
CPPYC

SEQ ID NO: 203 - protein, 5 aa, Artificial Sequence
  synthetic peptide
CSPYC

SEQ ID NO: 204 - protein, 5 aa, Artificial Sequence
  synthetic peptide
CTPYC

SEQ ID NO: 205 - protein, 5 aa, Artificial Sequence
  synthetic peptide
CCPYC

SEQ ID NO: 206 - protein, 5 aa, Artificial Sequence
  synthetic peptide
CYPYC

SEQ ID NO: 207 - protein, 5 aa, Artificial Sequence
  synthetic peptide
CNPYC

SEQ ID NO: 208 - protein, 5 aa, Artificial Sequence
  synthetic peptide
CQPYC

SEQ ID NO: 209 - protein, 5 aa, Artificial Sequence
  synthetic peptide
CDPYC

SEQ ID NO: 210 - protein, 5 aa, Artificial Sequence
  synthetic peptide
CEPYC

SEQ ID NO: 211 - protein, 5 aa, Artificial Sequence
  synthetic peptide
CPKYC

SEQ ID NO: 212 - protein, 5 aa, Artificial Sequence
  synthetic peptide
CPRYC

SEQ ID NO: 213 - protein, 5 aa, Artificial Sequence
  synthetic peptide
CPHYC

SEQ ID NO: 214 - protein, 5 aa, Artificial Sequence
  synthetic peptide
CPGYC

SEQ ID NO: 215 - protein, 5 aa, Artificial Sequence
  synthetic peptide
CPAYC

SEQ ID NO: 216 - protein, 5 aa, Artificial Sequence
  synthetic peptide
CPVYC

SEQ ID NO: 217 - protein, 5 aa, Artificial Sequence
  synthetic peptide
CPLYC

SEQ ID NO: 218 - protein, 5 aa, Artificial Sequence
  synthetic peptide
CPIYC

SEQ ID NO: 219 - protein, 5 aa, Artificial Sequence
  synthetic peptide
CPMYC

SEQ ID NO: 220 - protein, 5 aa, Artificial Sequence
  synthetic peptide
CPFYC

SEQ ID NO: 221 - protein, 5 aa, Artificial Sequence
  synthetic peptide
CPWYC

SEQ ID NO: 222 - protein, 5 aa, Artificial Sequence
  synthetic peptide
CPPYC

SEQ ID NO: 223 - protein, 5 aa, Artificial Sequence
  synthetic peptide
CPSYC

SEQ ID NO: 224 - protein, 5 aa, Artificial Sequence
  synthetic peptide
CPTYC

SEQ ID NO: 225 - protein, 5 aa, Artificial Sequence
  synthetic peptide
CPCYC

SEQ ID NO: 226 - protein, 5 aa, Artificial Sequence
  synthetic peptide
CPYYC

SEQ ID NO: 227 - protein, 5 aa, Artificial Sequence
  synthetic peptide
CPNYC

SEQ ID NO: 228 - protein, 5 aa, Artificial Sequence
  synthetic peptide
CPQYC

SEQ ID NO: 229 - protein, 5 aa, Artificial Sequence
  synthetic peptide
CPDYC

SEQ ID NO: 230 - protein, 5 aa, Artificial Sequence
  synthetic peptide
CPEYC

SEQ ID NO: 231 - protein, 5 aa, Artificial Sequence
  synthetic peptide
CPYKC

SEQ ID NO: 232 - protein, 5 aa, Artificial Sequence
  synthetic peptide
CPYRC

SEQ ID NO: 233 - protein, 5 aa, Artificial Sequence
  synthetic peptide
CPYHC

SEQ ID NO: 234 - protein, 5 aa, Artificial Sequence
  synthetic peptide
CPYGC

SEQ ID NO: 235 - protein, 5 aa, Artificial Sequence
  synthetic peptide
CPYAC

SEQ ID NO: 236 - protein, 5 aa, Artificial Sequence
  synthetic peptide
CPYVC

SEQ ID NO: 237 - protein, 5 aa, Artificial Sequence
  synthetic peptide
CPYLC

SEQ ID NO: 238 - protein, 5 aa, Artificial Sequence
  synthetic peptide
CPYIC

SEQ ID NO: 239 - protein, 5 aa, Artificial Sequence
  synthetic peptide
CPYMC

SEQ ID NO: 240 - protein, 5 aa, Artificial Sequence
  synthetic peptide
CPYFC

SEQ ID NO: 241 - protein, 5 aa, Artificial Sequence
  synthetic peptide
CPYWC

SEQ ID NO: 242 - protein, 5 aa, Artificial Sequence
  synthetic peptide
CPYPC

SEQ ID NO: 243 - protein, 5 aa, Artificial Sequence
  synthetic peptide
CPYSC

SEQ ID NO: 244 - protein, 5 aa, Artificial Sequence
  synthetic peptide
CPYTC

SEQ ID NO: 245 - protein, 5 aa, Artificial Sequence
  synthetic peptide
CPYCC

SEQ ID NO: 246 - protein, 5 aa, Artificial Sequence
  synthetic peptide
CPYYC

SEQ ID NO: 247 - protein, 5 aa, Artificial Sequence
  synthetic peptide
CPYNC

SEQ ID NO: 248 - protein, 5 aa, Artificial Sequence
  synthetic peptide
CPYQC

SEQ ID NO: 249 - protein, 5 aa, Artificial Sequence
  synthetic peptide
CPYDC

SEQ ID NO: 250 - protein, 5 aa, Artificial Sequence
  synthetic peptide
CPYEC

SEQ ID NO: 251 - protein, 5 aa, Artificial Sequence
  synthetic peptide
CPYLC

SEQ ID NO: 252 - protein, 5 aa, Artificial Sequence
  synthetic peptide
  misc_feature (2)..(2): Xaa can be any naturally occurring amino acid
CXHGC

SEQ ID NO: 253 - protein, 5 aa, Artificial Sequence
  synthetic peptide
  misc_feature (3)..(3): Xaa can be any naturally occurring amino acid
CHXGC

SEQ ID NO: 254 - protein, 5 aa, Artificial Sequence
  synthetic peptide
  misc_feature (4)..(4): Xaa can be any naturally occurring amino acid
CHGXC

SEQ ID NO: 255 - protein, 5 aa, Artificial Sequence
  synthetic peptide
CKHGC

SEQ ID NO: 256 - protein, 5 aa, Artificial Sequence
  synthetic peptide
CRHGC

SEQ ID NO: 257 - protein, 5 aa, Artificial Sequence
  synthetic peptide
CHHGC

SEQ ID NO: 258 - protein, 5 aa, Artificial Sequence
  synthetic peptide
CGHGC

SEQ ID NO: 259 - protein, 5 aa, Artificial Sequence
  synthetic peptide
CAHGC

SEQ ID NO: 260 - protein, 5 aa, Artificial Sequence
  synthetic peptide
CVHGC

SEQ ID NO: 261 - protein, 5 aa, Artificial Sequence
  synthetic peptide
CLHGC

SEQ ID NO: 262 - protein, 5 aa, Artificial Sequence
  synthetic peptide
CIHGC

SEQ ID NO: 263 - protein, 5 aa, Artificial Sequence
  synthetic peptide
CMHGC

SEQ ID NO: 264 - protein, 5 aa, Artificial Sequence
  synthetic peptide
CFHGC

SEQ ID NO: 265 - protein, 5 aa, Artificial Sequence
  synthetic peptide
CWHGC

SEQ ID NO: 266 - protein, 5 aa, Artificial Sequence
  synthetic peptide
CPHGC

SEQ ID NO: 267 - protein, 5 aa, Artificial Sequence
  synthetic peptide
CSHGC

SEQ ID NO: 268 - protein, 5 aa, Artificial Sequence
  synthetic peptide
CTHGC

SEQ ID NO: 269 - protein, 5 aa, Artificial Sequence
  synthetic peptide
CCHGC

SEQ ID NO: 270 - protein, 5 aa, Artificial Sequence
  synthetic peptide
CYHGC

SEQ ID NO: 271 - protein, 5 aa, Artificial Sequence
  synthetic peptide
CNHGC

SEQ ID NO: 272 - protein, 5 aa, Artificial Sequence
  synthetic peptide
CQHGC

SEQ ID NO: 273 - protein, 5 aa, Artificial Sequence
  synthetic peptide
CDHGC

SEQ ID NO: 274 - protein, 5 aa, Artificial Sequence
  synthetic peptide
CEHGC

SEQ ID NO: 275 - protein, 5 aa, Artificial Sequence
  synthetic peptide
CGKHC

SEQ ID NO: 276 - protein, 5 aa, Artificial Sequence
  synthetic peptide
CGRHC

SEQ ID NO: 277 - protein, 5 aa, Artificial Sequence
  synthetic peptide
CGHHC

SEQ ID NO: 278 - protein, 5 aa, Artificial Sequence
  synthetic peptide
CGGHC

SEQ ID NO: 279 - protein, 5 aa, Artificial Sequence
  synthetic peptide
CGAHC

SEQ ID NO: 280 - protein, 5 aa, Artificial Sequence
  synthetic peptide
CGVHC

SEQ ID NO: 281 - protein, 5 aa, Artificial Sequence
  synthetic peptide
CGLHC

SEQ ID NO: 282 - protein, 5 aa, Artificial Sequence
  synthetic peptide
CGIHC

SEQ ID NO: 283 - protein, 5 aa, Artificial Sequence
  synthetic peptide
CGMHC

SEQ ID NO: 284 - protein, 5 aa, Artificial Sequence
  synthetic peptide
CGFHC

SEQ ID NO: 285 - protein, 5 aa, Artificial Sequence
  synthetic peptide
CGWHC

SEQ ID NO: 286 - protein, 5 aa, Artificial Sequence
  synthetic peptide
CGPHC

SEQ ID NO: 287 - protein, 5 aa, Artificial Sequence
  synthetic peptide
CGSHC

SEQ ID NO: 288 - protein, 5 aa, Artificial Sequence
  synthetic peptide
CGTHC

SEQ ID NO: 289 - protein, 5 aa, Artificial Sequence
  synthetic peptide
CGCHC

SEQ ID NO: 290 - protein, 5 aa, Artificial Sequence
  synthetic peptide
CGYHC

SEQ ID NO: 291 - protein, 5 aa, Artificial Sequence
  synthetic peptide
CGNHC

SEQ ID NO: 292 - protein, 5 aa, Artificial Sequence
  synthetic peptide
CGQHC

SEQ ID NO: 293 - protein, 5 aa, Artificial Sequence
  synthetic peptide
CGDHC

SEQ ID NO: 294 - protein, 5 aa, Artificial Sequence
  synthetic peptide
CGEHC

SEQ ID NO: 295 - protein, 5 aa, Artificial Sequence
  synthetic peptide
CHGKC

SEQ ID NO: 296 - protein, 5 aa, Artificial Sequence
  synthetic peptide
CHGRC

SEQ ID NO: 297 - protein, 5 aa, Artificial Sequence
  synthetic peptide
CHGHC

SEQ ID NO: 298 - protein, 5 aa, Artificial Sequence
  synthetic peptide
CHGGC

SEQ ID NO: 299 - protein, 5 aa, Artificial Sequence
  synthetic peptide
CHGAC

SEQ ID NO: 300 - protein, 5 aa, Artificial Sequence
  synthetic peptide
CHGVC

SEQ ID NO: 301 - protein, 5 aa, Artificial Sequence
  synthetic peptide
CHGLC

SEQ ID NO: 302 - protein, 5 aa, Artificial Sequence
  synthetic peptide
CHGIC

SEQ ID NO: 303 - protein, 5 aa, Artificial Sequence
  synthetic peptide
CHGMC

SEQ ID NO: 304 - protein, 5 aa, Artificial Sequence
  synthetic peptide
CHGFC

SEQ ID NO: 305 - protein, 5 aa, Artificial Sequence
  synthetic peptide
CHGWC

SEQ ID NO: 306 - protein, 5 aa, Artificial Sequence
  synthetic peptide
CHGPC

SEQ ID NO: 307 - protein, 5 aa, Artificial Sequence
  synthetic peptide
CHGSC

SEQ ID NO: 308 - protein, 5 aa, Artificial Sequence
  synthetic peptide
CHGTC

SEQ ID NO: 309 - protein, 5 aa, Artificial Sequence
  synthetic peptide
CHGCC

SEQ ID NO: 310 - protein, 5 aa, Artificial Sequence
  synthetic peptide
CHGYC

SEQ ID NO: 311 - protein, 5 aa, Artificial Sequence
  synthetic peptide
CHGNC

SEQ ID NO: 312 - protein, 5 aa, Artificial Sequence
  synthetic peptide
CHGQC

SEQ ID NO: 313 - protein, 5 aa, Artificial Sequence
  synthetic peptide
CHGDC

SEQ ID NO: 314 - protein, 5 aa, Artificial Sequence
  synthetic peptide
CHGEC

SEQ ID NO: 315 - protein, 5 aa, Artificial Sequence
  synthetic peptide
  misc_feature (2)..(2): Xaa can be any naturally occurring amino acid
CXGPC

SEQ ID NO: 316 - protein, 5 aa, Artificial Sequence
  synthetic peptide
  misc_feature (3)..(3): Xaa can be any naturally occurring amino acid
CGXPC

SEQ ID NO: 317 - protein, 5 aa, Artificial Sequence
  synthetic peptide
  misc_feature (4)..(4): Xaa can be any naturally occurring amino acid
CGPXC

SEQ ID NO: 318 - protein, 5 aa, Artificial Sequence
  synthetic peptide
CKGPC

SEQ ID NO: 319 - protein, 5 aa, Artificial Sequence
  synthetic peptide
CRGPC

SEQ ID NO: 320 - protein, 5 aa, Artificial Sequence
  synthetic peptide
CHGPC

SEQ ID NO: 321 - protein, 5 aa, Artificial Sequence
  synthetic peptide
CGGPC

SEQ ID NO: 322 - protein, 5 aa, Artificial Sequence
  synthetic peptide
CAGPC

SEQ ID NO: 323 - protein, 5 aa, Artificial Sequence
  synthetic peptide
CVGPC

SEQ ID NO: 324 - protein, 5 aa, Artificial Sequence
  synthetic peptide
CLGPC

SEQ ID NO: 325 - protein, 5 aa, Artificial Sequence
  synthetic peptide
CIGPC

SEQ ID NO: 326 - protein, 5 aa, Artificial Sequence
  synthetic peptide
CMGPC

SEQ ID NO: 327 - protein, 5 aa, Artificial Sequence
  synthetic peptide
CFGPC

SEQ ID NO: 328 - protein, 5 aa, Artificial Sequence
  synthetic peptide
CWGPC

SEQ ID NO: 329 - protein, 5 aa, Artificial Sequence
  synthetic peptide
CPGPC

SEQ ID NO: 330 - protein, 5 aa, Artificial Sequence
  synthetic peptide
CSGPC

SEQ ID NO: 331 - protein, 5 aa, Artificial Sequence
  synthetic peptide
CTGPC

SEQ ID NO: 332 - protein, 5 aa, Artificial Sequence
  synthetic peptide
CCGPC

SEQ ID NO: 333 - protein, 5 aa, Artificial Sequence
  synthetic peptide
CYGPC

SEQ ID NO: 334 - protein, 5 aa, Artificial Sequence
  synthetic peptide
CNGPC

SEQ ID NO: 335 - protein, 5 aa, Artificial Sequence
  synthetic peptide
CQGPC

SEQ ID NO: 336 - protein, 5 aa, Artificial Sequence
  synthetic peptide
CDGPC

SEQ ID NO: 337 - protein, 5 aa, Artificial Sequence
  synthetic peptide
CEGPC

SEQ ID NO: 338 - protein, 5 aa, Artificial Sequence
  synthetic peptide
CGKPC

SEQ ID NO: 339 - protein, 5 aa, Artificial Sequence
  synthetic peptide
CGRPC

SEQ ID NO: 340 - protein, 5 aa, Artificial Sequence
  synthetic peptide
CGHPC

SEQ ID NO: 341 - protein, 5 aa, Artificial Sequence
  synthetic peptide
CGGPC

SEQ ID NO: 342 - protein, 5 aa, Artificial Sequence
  synthetic peptide
CGAPC

SEQ ID NO: 343 - protein, 5 aa, Artificial Sequence
  synthetic peptide
CGVPC

SEQ ID NO: 344 - protein, 5 aa, Artificial Sequence
  synthetic peptide
CGLPC

SEQ ID NO: 345 - protein, 5 aa, Artificial Sequence
  synthetic peptide
CGIPC

SEQ ID NO: 346 - protein, 5 aa, Artificial Sequence
  synthetic peptide
CGMPC

SEQ ID NO: 347 - protein, 5 aa, Artificial Sequence
  synthetic peptide
CGFPC

SEQ ID NO: 348 - protein, 5 aa, Artificial Sequence
  synthetic peptide
CGWPC

SEQ ID NO: 349 - protein, 5 aa, Artificial Sequence
  synthetic peptide
CGPPC

SEQ ID NO: 350 - protein, 5 aa, Artificial Sequence
  synthetic peptide
CGSPC

SEQ ID NO: 351 - protein, 5 aa, Artificial Sequence
  synthetic peptide
CGTPC

SEQ ID NO: 352 - protein, 5 aa, Artificial Sequence
  synthetic peptide
CGCPC

SEQ ID NO: 353 - protein, 5 aa, Artificial Sequence
  synthetic peptide
CGYPC

SEQ ID NO: 354 - protein, 5 aa, Artificial Sequence
  synthetic peptide
CGNPC

SEQ ID NO: 355 - protein, 5 aa, Artificial Sequence
  synthetic peptide
CGQPC

SEQ ID NO: 356 - protein, 5 aa, Artificial Sequence
  synthetic peptide
CGDPC

SEQ ID NO: 357 - protein, 5 aa, Artificial Sequence
  synthetic peptide
CGEPC

SEQ ID NO: 358 - protein, 5 aa, Artificial Sequence
  synthetic peptide
CGPKC

SEQ ID NO: 359 - protein, 5 aa, Artificial Sequence
  synthetic peptide
CGPRC

SEQ ID NO: 360 - protein, 5 aa, Artificial Sequence
  synthetic peptide
CGPHC

SEQ ID NO: 361 - protein, 5 aa, Artificial Sequence
  synthetic peptide
CGPGC

SEQ ID NO: 362 - protein, 5 aa, Artificial Sequence
  synthetic peptide
CGPAC

SEQ ID NO: 363 - protein, 5 aa, Artificial Sequence
  synthetic peptide
CGPVC

SEQ ID NO: 364 - protein, 5 aa, Artificial Sequence
  synthetic peptide
CGPLC

SEQ ID NO: 365 - protein, 5 aa, Artificial Sequence
  synthetic peptide
CGPIC

SEQ ID NO: 366 - protein, 5 aa, Artificial Sequence
  synthetic peptide
CGPMC

SEQ ID NO: 367 - protein, 5 aa, Artificial Sequence
  synthetic peptide
CGPFC

SEQ ID NO: 368 - protein, 5 aa, Artificial Sequence
  synthetic peptide
CGPWC

SEQ ID NO: 369 - protein, 5 aa, Artificial Sequence
  synthetic peptide
CGPPC

SEQ ID NO: 370 - protein, 5 aa, Artificial Sequence
  synthetic peptide
CGPSC

SEQ ID NO: 371 - protein, 5 aa, Artificial Sequence
  synthetic peptide
CGPTC

SEQ ID NO: 372 - protein, 5 aa, Artificial Sequence
  synthetic peptide
CGPCC

SEQ ID NO: 373 - protein, 5 aa, Artificial Sequence
  synthetic peptide
CGPYC

SEQ ID NO: 374 - protein, 5 aa, Artificial Sequence
  synthetic peptide
CGPNC

SEQ ID NO: 375 - protein, 5 aa, Artificial Sequence
  synthetic peptide
CGPQC

SEQ ID NO: 376 - protein, 5 aa, Artificial Sequence
  synthetic peptide
CGPDC

SEQ ID NO: 377 - protein, 5 aa, Artificial Sequence
  synthetic peptide
CGPEC

SEQ ID NO: 378 - protein, 5 aa, Artificial Sequence
  synthetic peptide
  misc_feature (2)..(2): Xaa can be any naturally occurring amino acid
CXGHC

SEQ ID NO: 379 - protein, 5 aa, Artificial Sequence
  synthetic peptide
  misc_feature (3)..(3): Xaa can be any naturally occurring amino acid
CGXHC

SEQ ID NO: 380 - protein, 5 aa, Artificial Sequence
  synthetic peptide
  misc_feature (4)..(4): Xaa can be any naturally occurring amino acid
CGHXC

SEQ ID NO: 381 - protein, 5 aa, Artificial Sequence
  synthetic peptide
CKGHC

SEQ ID NO: 382 - protein, 5 aa, Artificial Sequence
  synthetic peptide
CRGHC

SEQ ID NO: 383 - protein, 5 aa, Artificial Sequence
  synthetic peptide
CHGHC

SEQ ID NO: 384 - protein, 5 aa, Artificial Sequence
  synthetic peptide
CGGHC

SEQ ID NO: 385 - protein, 5 aa, Artificial Sequence
  synthetic peptide
CAGHC

SEQ ID NO: 386 - protein, 5 aa, Artificial Sequence
  synthetic peptide
CVGHC

SEQ ID NO: 387 - protein, 5 aa, Artificial Sequence
  synthetic peptide
CLGHC

SEQ ID NO: 388 - protein, 5 aa, Artificial Sequence
  synthetic peptide
CIGHC

SEQ ID NO: 389 - protein, 5 aa, Artificial Sequence
  synthetic peptide
CMGHC

SEQ ID NO: 390 - protein, 5 aa, Artificial Sequence
  synthetic peptide
CFGHC

SEQ ID NO: 391 - protein, 5 aa, Artificial Sequence
  synthetic peptide
CWGHC

SEQ ID NO: 392 - protein, 5 aa, Artificial Sequence
  synthetic peptide
CPGHC

SEQ ID NO: 393 - protein, 5 aa, Artificial Sequence
  synthetic peptide
CSGHC

SEQ ID NO: 394 - protein, 5 aa, Artificial Sequence
  synthetic peptide
CTGHC

SEQ ID NO: 395 - protein, 5 aa, Artificial Sequence
  synthetic peptide
CCGHC

SEQ ID NO: 396 - protein, 5 aa, Artificial Sequence
  synthetic peptide
CYGHC

SEQ ID NO: 397 - protein, 5 aa, Artificial Sequence
  synthetic peptide
CNGHC

SEQ ID NO: 398 - protein, 5 aa, Artificial Sequence
  synthetic peptides
CQGHC

SEQ ID NO: 399 - protein, 5 aa, Artificial Sequence
  synthetic peptide
CDGHC

SEQ ID NO: 400 - protein, 5 aa, Artificial Sequence
  synthetic peptide
CEGHC

SEQ ID NO: 401 - protein, 5 aa, Artificial Sequence
  synthetic peptide
  misc_feature (3)..(3): Xaa can be any naturally occurring amino acid
CGXFC

SEQ ID NO: 402 - protein, 5 aa, Artificial Sequence
  synthetic peptide
CGKFC

SEQ ID NO: 403 - protein, 5 aa, Artificial Sequence
  synthetic peptide
CGRFC

SEQ ID NO: 404 - protein, 5 aa, Artificial Sequence
  synthetic peptide
CGHFC

SEQ ID NO: 405 - protein, 5 aa, Artificial Sequence
  synthetic peptide
CGGFC

SEQ ID NO: 406 - protein, 5 aa, Artificial Sequence
  synthetic peptide
CGAFC

SEQ ID NO: 407 - protein, 5 aa, Artificial Sequence
  synthetic peptide
CGVFC

SEQ ID NO: 408 - protein, 5 aa, Artificial Sequence
  synthetic peptide
CGLFC

SEQ ID NO: 409 - protein, 5 aa, Artificial Sequence
  synthetic peptide
CGIFC

SEQ ID NO: 410 - protein, 5 aa, Artificial Sequence
  synthetic peptide
CGMFC

SEQ ID NO: 411 - protein, 5 aa, Artificial Sequence
  synthetic peptide
CGFFC

SEQ ID NO: 412 - protein, 5 aa, Artificial Sequence
  synthetic peptide
CGWFC

SEQ ID NO: 413 - protein, 5 aa, Artificial Sequence
  synthetic peptide
CGSFC

SEQ ID NO: 414 - protein, 5 aa, Artificial Sequence
  synthetic peptide
CGTFC

SEQ ID NO: 415 - protein, 5 aa, Artificial Sequence
  synthetic peptide
CGCFC

SEQ ID NO: 416 - protein, 5 aa, Artificial Sequence
  synthetic peptide
CGYFC

SEQ ID NO: 417 - protein, 5 aa, Artificial Sequence
  synthetic peptide
CGNFC

SEQ ID NO: 418 - protein, 5 aa, Artificial Sequence
  synthetic peptide
CGQFC

SEQ ID NO: 419 - protein, 5 aa, Artificial Sequence
  synthetic peptide
CGDFC

SEQ ID NO: 420 - protein, 5 aa, Artificial Sequence
  synthetic peptide
CGEFC

SEQ ID NO: 421 - protein, 5 aa, Artificial Sequence
  synthetic peptide
CGHKC

SEQ ID NO: 422 - protein, 5 aa, Artificial Sequence
  synthetic peptide
CGHRC

SEQ ID NO: 423 - protein, 5 aa, Artificial Sequence
  synthetic peptide
CGHHC

SEQ ID NO: 424 - protein, 5 aa, Artificial Sequence
  synthetic peptide
CGHGC

SEQ ID NO: 425 - protein, 5 aa, Artificial Sequence
  synthetic peptide
CGHAC

SEQ ID NO: 426 - protein, 5 aa, Artificial Sequence
  synthetic peptide
CGHVC

SEQ ID NO: 427 - protein, 5 aa, Artificial Sequence
  synthetic peptide
CGHLC

SEQ ID NO: 428 - protein, 5 aa, Artificial Sequence
  synthetic peptide
CGHIC

SEQ ID NO: 429 - protein, 5 aa, Artificial Sequence
  synthetic peptide
CGHMC

SEQ ID NO: 430 - protein, 5 aa, Artificial Sequence
  synthetic peptide
CGHWC

SEQ ID NO: 431 - protein, 5 aa, Artificial Sequence
  synthetic peptide
CGHPC

SEQ ID NO: 432 - protein, 5 aa, Artificial Sequence
  synthetic peptide
CGHSC

SEQ ID NO: 433 - protein, 5 aa, Artificial Sequence
  synthetic peptide
CGHTC

SEQ ID NO: 434 - protein, 5 aa, Artificial Sequence
  synthetic peptide
CGHCC

SEQ ID NO: 435 - protein, 5 aa, Artificial Sequence
  synthetic peptide
CGHYC

SEQ ID NO: 436 - protein, 5 aa, Artificial Sequence
  synthetic peptide
CGHNC

SEQ ID NO: 437 - protein, 5 aa, Artificial Sequence
  synthetic peptide
CGHQC

SEQ ID NO: 438 - protein, 5 aa, Artificial Sequence
  synthetic peptide
CGHDC

SEQ ID NO: 439 - protein, 5 aa, Artificial Sequence
  synthetic peptide
CGHEC

SEQ ID NO: 440 - protein, 5 aa, Artificial Sequence
  synthetic peptide
  misc_feature (2)..(2): Xaa can be any naturally occurring amino acid
CXGFC

SEQ ID NO: 441 - protein, 5 aa, Artificial Sequence
  synthetic peptide
  misc_feature (4)..(4): Xaa can be any naturally occurring amino acid
CGFXC

SEQ ID NO: 442 - protein, 5 aa, Artificial Sequence
  synthetic peptide
CKGFC

SEQ ID NO: 443 - protein, 5 aa, Artificial Sequence
  synthetic peptide
CRGFC

SEQ ID NO: 444 - protein, 5 aa, Artificial Sequence
  synthetic peptide
CHGFC

SEQ ID NO: 445 - protein, 5 aa, Artificial Sequence
  synthetic peptide
CAGFC

SEQ ID NO: 446 - protein, 5 aa, Artificial Sequence
  synthetic peptide
CVGFC

SEQ ID NO: 447 - protein, 5 aa, Artificial Sequence
  synthetic peptide
CLGFC

SEQ ID NO: 448 - protein, 5 aa, Artificial Sequence
  synthetic peptide
CIGFC

SEQ ID NO: 449 - protein, 5 aa, Artificial Sequence
  synthetic peptide
CMGFC

SEQ ID NO: 450 - protein, 5 aa, Artificial Sequence
  synthetic peptide
CFGFC

SEQ ID NO: 451 - protein, 5 aa, Artificial Sequence
  synthetic peptide
CWGFC

SEQ ID NO: 452 - protein, 5 aa, Artificial Sequence
  synthetic peptide
CPGFC

SEQ ID NO: 453 - protein, 5 aa, Artificial Sequence
  synthetic peptide
CSGFC

SEQ ID NO: 454 - protein, 5 aa, Artificial Sequence
  synthetic peptide
CTGFC

SEQ ID NO: 455 - protein, 5 aa, Artificial Sequence
  synthetic peptide
CCGFC

SEQ ID NO: 456 - protein, 5 aa, Artificial Sequence
  synthetic peptide
CYGFC

SEQ ID NO: 457 - protein, 5 aa, Artificial Sequence
  synthetic peptide
CNGFC

SEQ ID NO: 458 - protein, 5 aa, Artificial Sequence
  synthetic peptide
CQGFC

SEQ ID NO: 459 - protein, 5 aa, Artificial Sequence
  synthetic peptide
CDGFC

SEQ ID NO: 460 - protein, 5 aa, Artificial Sequence
  synthetic peptide
CEGFC

SEQ ID NO: 461 - protein, 5 aa, Artificial Sequence
  synthetic peptide
CGFKC

SEQ ID NO: 462 - protein, 5 aa, Artificial Sequence
  synthetic peptide
CGFRC

SEQ ID NO: 463 - protein, 5 aa, Artificial Sequence
  synthetic peptide
CGFHC

SEQ ID NO: 464 - protein, 5 aa, Artificial Sequence
  synthetic peptide
CGFGC

SEQ ID NO: 465 - protein, 5 aa, Artificial Sequence
  synthetic peptide
CGFAC

SEQ ID NO: 466 - protein, 5 aa, Artificial Sequence
  synthetic peptide
CGFVC

SEQ ID NO: 467 - protein, 5 aa, Artificial Sequence
  synthetic peptide
CGFLC

SEQ ID NO: 468 - protein, 5 aa, Artificial Sequence
  synthetic peptide
CGFIC

SEQ ID NO: 469 - protein, 5 aa, Artificial Sequence
  synthetic peptide
CGFMC

SEQ ID NO: 470 - protein, 5 aa, Artificial Sequence
  synthetic peptide
CGFWC

SEQ ID NO: 471 - protein, 5 aa, Artificial Sequence
  synthetic peptide
CGFPC

SEQ ID NO: 472 - protein, 5 aa, Artificial Sequence
  synthetic peptide
CGFSC

SEQ ID NO: 473 - protein, 5 aa, Artificial Sequence
  synthetic peptide
CGFTC

SEQ ID NO: 474 - protein, 5 aa, Artificial Sequence
  synthetic peptide
CGFCC

SEQ ID NO: 475 - protein, 5 aa, Artificial Sequence
  synthetic peptide
CGFYC

SEQ ID NO: 476 - protein, 5 aa, Artificial Sequence
  synthetic peptide
CGFNC

SEQ ID NO: 477 - protein, 5 aa, Artificial Sequence
  synthetic peptide
CGFQC

SEQ ID NO: 478 - protein, 5 aa, Artificial Sequence
  synthetic peptide
CGFDC

SEQ ID NO: 479 - protein, 5 aa, Artificial Sequence
  synthetic peptide
CGFEC

SEQ ID NO: 480 - protein, 5 aa, Artificial Sequence
  synthetic peptide
  misc_feature (2)..(2): Xaa can be any naturally occurring amino acid
CXRLC

SEQ ID NO: 481 - protein, 5 aa, Artificial Sequence
  synthetic peptide
  misc_feature (3)..(3): Xaa can be any naturally occurring amino acid
CRXLC

SEQ ID NO: 482 - protein, 5 aa, Artificial Sequence
  synthetic peptide
  misc_feature (4)..(4): Xaa can be any naturally occurring amino acid
CRLXC

SEQ ID NO: 483 - protein, 5 aa, Artificial Sequence
  synthetic peptide
CKRLC

SEQ ID NO: 484 - protein, 5 aa, Artificial Sequence
  synthetic peptide
CRRLC

SEQ ID NO: 485 - protein, 5 aa, Artificial Sequence
  synthetic peptide
CHRLC

SEQ ID NO: 486 - protein, 5 aa, Artificial Sequence
  synthetic peptide
CGRLC

SEQ ID NO: 487 - protein, 5 aa, Artificial Sequence
  synthetic peptide
CARLC

SEQ ID NO: 488 - protein, 5 aa, Artificial Sequence
  synthetic peptide
CVRLC

SEQ ID NO: 489 - protein, 5 aa, Artificial Sequence
  synthetic peptide
CLRLC

SEQ ID NO: 490 - protein, 5 aa, Artificial Sequence
  synthetic peptide
CIRLC

SEQ ID NO: 491 - protein, 5 aa, Artificial Sequence
  synthetic peptide
CMRLC

SEQ ID NO: 492 - protein, 5 aa, Artificial Sequence
  synthetic peptide
CFRLC

SEQ ID NO: 493 - protein, 5 aa, Artificial Sequence
  synthetic peptide
CWRLC

SEQ ID NO: 494 - protein, 5 aa, Artificial Sequence
  synthetic peptide
CPRLC

SEQ ID NO: 495 - protein, 5 aa, Artificial Sequence
  synthetic peptide
CSRLC

SEQ ID NO: 496 - protein, 5 aa, Artificial Sequence
  synthetic peptide
CTRLC

SEQ ID NO: 497 - protein, 5 aa, Artificial Sequence
  synthetic peptide
CCRLC

SEQ ID NO: 498 - protein, 5 aa, Artificial Sequence
  synthetic peptide
CYRLC

SEQ ID NO: 499 - protein, 5 aa, Artificial Sequence
  synthetic peptide
CNRLC

SEQ ID NO: 500 - protein, 5 aa, Artificial Sequence
  synthetic peptide
CQRLC

SEQ ID NO: 501 - protein, 5 aa, Artificial Sequence
  synthetic peptide
CDRLC

SEQ ID NO: 502 - protein, 5 aa, Artificial Sequence
  synthetic peptide
CERLC

SEQ ID NO: 503 - protein, 5 aa, Artificial Sequence
  synthetic peptide
CRKLC

SEQ ID NO: 504 - protein, 5 aa, Artificial Sequence
  synthetic peptide
CRRLC

SEQ ID NO: 505 - protein, 5 aa, Artificial Sequence
  synthetic peptide
CRHLC

SEQ ID NO: 506 - protein, 5 aa, Artificial Sequence
  synthetic peptide
CRGLC

SEQ ID NO: 507 - protein, 5 aa, Artificial Sequence
  synthetic peptide
CRALC

SEQ ID NO: 508 - protein, 5 aa, Artificial Sequence
  synthetic peptide
CRVLC

SEQ ID NO: 509 - protein, 5 aa, Artificial Sequence
  synthetic peptide
CRLLC

SEQ ID NO: 510 - protein, 5 aa, Artificial Sequence
  synthetic peptide
CRILC

SEQ ID NO: 511 - protein, 5 aa, Artificial Sequence
  synthetic peptide
CRMLC

SEQ ID NO: 512 - protein, 5 aa, Artificial Sequence
  synthetic peptide
CRFLC

SEQ ID NO: 513 - protein, 5 aa, Artificial Sequence
  synthetic peptide
CRWLC

SEQ ID NO: 514 - protein, 5 aa, Artificial Sequence
  synthetic peptide
CRPLC

SEQ ID NO: 515 - protein, 5 aa, Artificial Sequence
  synthetic peptide
CRSLC

SEQ ID NO: 516 - protein, 5 aa, Artificial Sequence
  synthetic peptide
CRTLC

SEQ ID NO: 517 - protein, 5 aa, Artificial Sequence
  synthetic peptide
CRCLC

SEQ ID NO: 518 - protein, 5 aa, Artificial Sequence
  synthetic peptide
CRYLC

SEQ ID NO: 519 - protein, 5 aa, Artificial Sequence
  synthetic peptide
CRNLC

SEQ ID NO: 520 - protein, 5 aa, Artificial Sequence
  synthetic peptide
CRQLC

SEQ ID NO: 521 - protein, 5 aa, Artificial Sequence
  synthetic peptide
CRDLC

SEQ ID NO: 522 - protein, 5 aa, Artificial Sequence
  synthetic peptide
CRELC

SEQ ID NO: 523 - protein, 5 aa, Artificial Sequence
  synthetic peptide
CRLKC

SEQ ID NO: 524 - protein, 5 aa, Artificial Sequence
  synthetic peptide
CRLRC

SEQ ID NO: 525 - protein, 5 aa, Artificial Sequence
  synthetic peptide
CRLHC

SEQ ID NO: 526 - protein, 5 aa, Artificial Sequence
  synthetic peptide
CRLGC

SEQ ID NO: 527 - protein, 5 aa, Artificial Sequence
  synthetic peptide
CRLAC

SEQ ID NO: 528 - protein, 5 aa, Artificial Sequence
  synthetic peptide
CRLVC

SEQ ID NO: 529 - protein, 5 aa, Artificial Sequence
  synthetic peptide
CRLIC

SEQ ID NO: 530 - protein, 5 aa, Artificial Sequence
  synthetic peptide
CRLMC

SEQ ID NO: 531 - protein, 5 aa, Artificial Sequence
  synthetic peptide
CRLFC

SEQ ID NO: 532 - protein, 5 aa, Artificial Sequence
  synthetic peptide
CRLWC

SEQ ID NO: 533 - protein, 5 aa, Artificial Sequence
  synthetic peptide
CRLPC

SEQ ID NO: 534 - protein, 5 aa, Artificial Sequence
  synthetic peptide
CRLSC

SEQ ID NO: 535 - protein, 5 aa, Artificial Sequence
  synthetic peptide
CRLTC

SEQ ID NO: 536 - protein, 5 aa, Artificial Sequence
  synthetic peptide
CRLCC

SEQ ID NO: 537 - protein, 5 aa, Artificial Sequence
  synthetic peptide
CRLYC

SEQ ID NO: 538 - protein, 5 aa, Artificial Sequence
  synthetic peptide
CRLNC

SEQ ID NO: 539 - protein, 5 aa, Artificial Sequence
  synthetic peptide
CRLQC

SEQ ID NO: 540 - protein, 5 aa, Artificial Sequence
  synthetic peptide
CRLDC

SEQ ID NO: 541 - protein, 5 aa, Artificial Sequence
  synthetic peptide
CRLEC

SEQ ID NO: 542 - protein, 5 aa, Artificial Sequence
  synthetic peptide
  misc_feature (2)..(2): Xaa can be any naturally occurring amino acid
CXHPC

SEQ ID NO: 543 - protein, 5 aa, Artificial Sequence
  synthetic peptide
  misc_feature (3)..(3): Xaa can be any naturally occurring amino acid
CHXPC

SEQ ID NO: 544 - protein, 5 aa, Artificial Sequence
  synthetic peptide
  misc_feature (4)..(4): Xaa can be any naturally occurring amino acid
CHPXC

SEQ ID NO: 545 - protein, 5 aa, Artificial Sequence
  synthetic peptide
CKHPC

SEQ ID NO: 546 - protein, 5 aa, Artificial Sequence
  synthetic peptide
CRHPC

SEQ ID NO: 547 - protein, 5 aa, Artificial Sequence
  synthetic peptide
CHHPC

SEQ ID NO: 548 - protein, 5 aa, Artificial Sequence
  synthetic peptide
CGHPC

SEQ ID NO: 549 - protein, 5 aa, Artificial Sequence
  synthetic peptide
CAHPC

SEQ ID NO: 550 - protein, 5 aa, Artificial Sequence
  synthetic peptide
CVHPC

SEQ ID NO: 551 - protein, 5 aa, Artificial Sequence
  synthetic peptide
CLHPC

SEQ ID NO: 552 - protein, 5 aa, Artificial Sequence
  synthetic peptide
CIHPC

SEQ ID NO: 553 - protein, 5 aa, Artificial Sequence
  synthetic peptide
CMHPC

SEQ ID NO: 554 - protein, 5 aa, Artificial Sequence
  synthetic peptide
CFHPC

SEQ ID NO: 555 - protein, 5 aa, Artificial Sequence
  synthetic peptide
CWHPC

SEQ ID NO: 556 - protein, 5 aa, Artificial Sequence
  synthetic peptide
CPHPC

SEQ ID NO: 557 - protein, 5 aa, Artificial Sequence
  synthetic peptide
CSHPC

SEQ ID NO: 558 - protein, 5 aa, Artificial Sequence
  synthetic peptide
CTHPC

SEQ ID NO: 559 - protein, 5 aa, Artificial Sequence
  synthetic peptide
CCHPC

SEQ ID NO: 560 - protein, 5 aa, Artificial Sequence
  synthetic peptide
CYHPC

SEQ ID NO: 561 - protein, 5 aa, Artificial Sequence
  synthetic peptide
CNHPC

SEQ ID NO: 562 - protein, 5 aa, Artificial Sequence
  synthetic peptide
CQHPC

SEQ ID NO: 563 - protein, 5 aa, Artificial Sequence
  synthetic peptide
CDHPC

SEQ ID NO: 564 - protein, 5 aa, Artificial Sequence
  synthetic peptide
CEHPC

SEQ ID NO: 565 - protein, 5 aa, Artificial Sequence
  synthetic peptide
CKHPC

SEQ ID NO: 566 - protein, 5 aa, Artificial Sequence
  synthetic peptide
CHKPC

SEQ ID NO: 567 - protein, 5 aa, Artificial Sequence
  synthetic peptide
CHRPC

SEQ ID NO: 568 - protein, 5 aa, Artificial Sequence
  synthetic peptide
CHHPC

SEQ ID NO: 569 - protein, 5 aa, Artificial Sequence
  synthetic peptide
CHGPC

SEQ ID NO: 570 - protein, 5 aa, Artificial Sequence
  synthetic peptide
CHAPC

SEQ ID NO: 571 - protein, 5 aa, Artificial Sequence
  synthetic peptide
CHVPC

SEQ ID NO: 572 - protein, 5 aa, Artificial Sequence
  synthetic peptide
CHLPC

SEQ ID NO: 573 - protein, 5 aa, Artificial Sequence
  synthetic peptide
CHIPC

SEQ ID NO: 574 - protein, 5 aa, Artificial Sequence
  synthetic peptide
CHMPC

SEQ ID NO: 575 - protein, 5 aa, Artificial Sequence
  synthetic peptide
CHFPC

SEQ ID NO: 576 - protein, 5 aa, Artificial Sequence
  synthetic peptide
CHWPC

SEQ ID NO: 577 - protein, 5 aa, Artificial Sequence
  synthetic peptide
CHPPC

SEQ ID NO: 578 - protein, 5 aa, Artificial Sequence
  synthetic peptide
CHSPC

SEQ ID NO: 579 - protein, 5 aa, Artificial Sequence
  synthetic peptide
CHTPC

SEQ ID NO: 580 - protein, 5 aa, Artificial Sequence
  synthetic peptide
CHCPC

SEQ ID NO: 581 - protein, 5 aa, Artificial Sequence
  synthetic peptide
CHYPC

SEQ ID NO: 582 - protein, 5 aa, Artificial Sequence
  synthetic peptide
CHNPC

SEQ ID NO: 583 - protein, 5 aa, Artificial Sequence
  synthetic peptide
CHQPC

SEQ ID NO: 584 - protein, 5 aa, Artificial Sequence
  synthetic peptide
CHDPC

SEQ ID NO: 585 - protein, 5 aa, Artificial Sequence
  synthetic peptide
CHEPC

SEQ ID NO: 586 - protein, 5 aa, Artificial Sequence
  synthetic peptide
CHPKC

SEQ ID NO: 587 - protein, 5 aa, Artificial Sequence
  synthetic peptide
CHPRC

SEQ ID NO: 588 - protein, 5 aa, Artificial Sequence
  synthetic peptide
CHPHC

SEQ ID NO: 589 - protein, 5 aa, Artificial Sequence
  synthetic peptide
CHPGC

SEQ ID NO: 590 - protein, 5 aa, Artificial Sequence
  synthetic peptide
CHPAC

SEQ ID NO: 591 - protein, 5 aa, Artificial Sequence
  synthetic peptide
CHPVC

SEQ ID NO: 592 - protein, 5 aa, Artificial Sequence
  synthetic peptide
CHPLC

SEQ ID NO: 593 - protein, 5 aa, Artificial Sequence
  synthetic peptide
CHPIC

SEQ ID NO: 594 - protein, 5 aa, Artificial Sequence
  synthetic peptide
CHPMC

SEQ ID NO: 595 - protein, 5 aa, Artificial Sequence
  synthetic peptide
CHPFC

SEQ ID NO: 596 - protein, 5 aa, Artificial Sequence
  synthetic peptide
CHPWC

SEQ ID NO: 597 - protein, 5 aa, Artificial Sequence
  synthetic peptide
CHPPC

SEQ ID NO: 598 - protein, 5 aa, Artificial Sequence
  synthetic peptide
CHPSC

SEQ ID NO: 599 - protein, 5 aa, Artificial Sequence
  synthetic peptide
CHPTC

SEQ ID NO: 600 - protein, 5 aa, Artificial Sequence
  synthetic peptide
CHPCC

SEQ ID NO: 601 - protein, 5 aa, Artificial Sequence
  synthetic peptide
CHPYC

SEQ ID NO: 602 - protein, 5 aa, Artificial Sequence
  synthetic peptide
CHPNC

SEQ ID NO: 603 - protein, 5 aa, Artificial Sequence
  synthetic peptide
CHPQC

SEQ ID NO: 604 - protein, 5 aa, Artificial Sequence
  synthetic peptide
CHPDC

SEQ ID NO: 605 - protein, 5 aa, Artificial Sequence
  synthetic peptide
CHPEC

SEQ ID NO: 606 - protein, 6 aa, Artificial Sequence
  synthetic peptide
  misc_feature (2)..(5): Xaa can be any naturally occurring amino acid
CXXXXC

SEQ ID NO: 607 - protein, 6 aa, Artificial Sequence
  synthetic peptide
CLAVLC

SEQ ID NO: 608 - protein, 6 aa, Artificial Sequence
  synthetic peptide
CTVQAC

SEQ ID NO: 609 - protein, 6 aa, Artificial Sequence
  synthetic peptide
CGAVHC

SEQ ID NO: 610 - protein, 6 aa, Artificial Sequence
  synthetic peptide
  misc_feature (2)..(2): Xaa can be any naturally occurring amino acid
CXAVLC

SEQ ID NO: 611 - protein, 6 aa, Artificial Sequence
  synthetic peptide
  misc_feature (3)..(3): Xaa can be any naturally occurring amino acid
CLXVLC

SEQ ID NO: 612 - protein, 6 aa, Artificial Sequence
  synthetic peptide
  misc_feature (4)..(4): Xaa can be any naturally occurring amino acid
CLAXLC

SEQ ID NO: 613 - protein, 6 aa, Artificial Sequence
  synthetic peptide
  misc_feature (5)..(5): Xaa can be any naturally occurring amino acid
CLAVXC

SEQ ID NO: 614 - protein, 6 aa, Artificial Sequence
  synthetic peptide
  misc_feature (2)..(2): Xaa can be any naturally occurring amino acid
CXVQAC

SEQ ID NO: 615 - protein, 6 aa, Artificial Sequence
  synthetic peptide
  misc_feature (3)..(3): Xaa can be any naturally occurring amino acid
CTXQAC

SEQ ID NO: 616 - protein, 6 aa, Artificial Sequence
  synthetic peptide
  misc_feature (4)..(4): Xaa can be any naturally occurring amino acid
CTVXAC

SEQ ID NO: 617 - protein, 6 aa, Artificial Sequence
  synthetic peptide
  misc_feature (5)..(5): Xaa can be any naturally occurring amino acid
CTVQXC

SEQ ID NO: 618 - protein, 6 aa, Artificial Sequence
  synthetic peptide
  misc_feature (2)..(2): Xaa can be any naturally occurring amino acid
CXAVHC

SEQ ID NO: 619 - protein, 6 aa, Artificial Sequence
  synthetic peptide
  misc_feature (3)..(3): Xaa can be any naturally occurring amino acid
CGXVHC

SEQ ID NO: 620 - protein, 6 aa, Artificial Sequence
  synthetic peptide
  misc_feature (4)..(4): Xaa can be any naturally occurring amino acid
CGAXHC

SEQ ID NO: 621 - protein, 6 aa, Artificial Sequence
  synthetic peptide
  misc_feature (5)..(5): Xaa can be any naturally occurring amino acid
CGAVXC

SEQ ID NO: 622 - protein, 7 aa, Artificial Sequence
  synthetic peptide
  misc_feature (2)..(6): Xaa can be any naturally occurring amino acid
CXXXXXC

SEQ ID NO: 623 - protein, 7 aa, Artificial Sequence
  synthetic peptide
CPAFPLC

SEQ ID NO: 624 - protein, 7 aa, Artificial Sequence
  synthetic peptide
CDQGGEC

SEQ ID NO: 625 - protein, 7 aa, Artificial Sequence
  synthetic peptide
  misc_feature (2)..(2): Xaa can be any naturally occurring amino acid
CXAFPLC

SEQ ID NO: 626 - protein, 7 aa, Artificial Sequence
  synthetic peptide
  misc_feature (3)..(3): Xaa can be any naturally occurring amino acid
CPXFPLC

SEQ ID NO: 627 - protein, 7 aa, Artificial Sequence
  synthetic peptide
  misc_feature (4)..(4): Xaa can be any naturally occurring amino acid
CPAXPLC

SEQ ID NO: 628 - protein, 7 aa, Artificial Sequence
  synthetic peptide
  misc_feature (5)..(5): Xaa can be any naturally occurring amino acid
CPAFXLC

SEQ ID NO: 629 - protein, 7 aa, Artificial Sequence
  synthetic peptide
  misc_feature (6)..(6): Xaa can be any naturally occurring amino acid
CPAFPXC

SEQ ID NO: 630 - protein, 7 aa, Artificial Sequence
  synthetic peptide
  misc_feature (2)..(2): Xaa can be any naturally occurring amino acid
CXQGGEC

SEQ ID NO: 631 - protein, 7 aa, Artificial Sequence
  synthetic peptide
  misc_feature (3)..(3): Xaa can be any naturally occurring amino acid
CDXGGEC

SEQ ID NO: 632 - protein, 7 aa, Artificial Sequence
  synthetic peptide
  misc_feature (4)..(4): Xaa can be any naturally occurring amino acid
CDQXGEC

SEQ ID NO: 633 - protein, 7 aa, Artificial Sequence
  synthetic peptide
  misc_feature (5)..(5): Xaa can be any naturally occurring amino acid
CDQGXEC

SEQ ID NO: 634 - protein, 7 aa, Artificial Sequence
  synthetic peptide
  misc_feature (6)..(6): Xaa can be any naturally occurring amino acid
CDQGGXC

SEQ ID NO: 635 - protein, 8 aa, Artificial Sequence
  synthetic peptide
  misc_feature (2)..(7): Xaa can be any naturally occurring amino acid
CXXXXXXC

SEQ ID NO: 636 - protein, 8 aa, Artificial Sequence
  synthetic peptide
CDIADKYC

SEQ ID NO: 637 - protein, 8 aa, Artificial Sequence
  synthetic peptide
  misc_feature (2)..(2): Xaa can be any naturally occurring amino acid
CXIADKYC

SEQ ID NO: 638 - protein, 8 aa, Artificial Sequence
  synthetic peptide
  misc_feature (3)..(3): Xaa can be any naturally occurring amino acid
CDXADKYC

SEQ ID NO: 639 - protein, 8 aa, Artificial Sequence
  synthetic peptide
  misc_feature (4)..(4): Xaa can be any naturally occurring amino acid
CDIXDKYC

SEQ ID NO: 640 - protein, 8 aa, Artificial Sequence
  synthetic peptide
  misc_feature (5)..(5): Xaa can be any naturally occurring amino acid
CDIAXKYC

SEQ ID NO: 641 - protein, 8 aa, Artificial Sequence
  synthetic peptide
  misc_feature (6)..(6): Xaa can be any naturally occurring amino acid
CDIADXYC

SEQ ID NO: 642 - protein, 8 aa, Artificial Sequence
  synthetic peptide
  misc_feature (7)..(7): Xaa can be any naturally occurring amino acid
CDIADKXC

SEQ ID NO: 643 - protein, 5 aa, Artificial Sequence
  synthetic peptide
  misc_feature (2)..(2): Xaa can be any naturally occurring amino acid
  PEPTIDE (3)..(3): K, H, or R
  misc_feature (4)..(4): Xaa can be any naturally occurring amino acid
CXXXC

SEQ ID NO: 644 - protein, 5 aa, Artificial Sequence
  synthetic peptide
  misc_feature (2)..(3): Xaa can be any naturally occurring amino acid
  PEPTIDE (4)..(4): K, H, or R
CXXXC

SEQ ID NO: 645 - protein, 4 aa, Artificial Sequence
  synthetic peptide
  PEPTIDE (1)..(1): K, H, or R
  misc_feature (3)..(3): Xaa can be any naturally occurring amino acid
XCXC

SEQ ID NO: 646 - protein, 5 aa, Artificial Sequence
  synthetic peptide
  PEPTIDE (1)..(1): K, H, or R
  misc_feature (2)..(4): Xaa can be any naturally occurring amino acid
XXXXC

SEQ ID NO: 647 - protein, 4 aa, Artificial Sequence
  synthetic peptide
  misc_feature (2)..(2): Xaa can be any naturally occurring amino acid
  PEPTIDE (4)..(4): K, H, or R
CXCX

SEQ ID NO: 648 - protein, 6 aa, Artificial Sequence
  synthetic peptide
  misc_feature (2)..(4): Xaa can be any naturally occurring amino acid
  PEPTIDE (6)..(6): K, H, or R
CXXXCX

SEQ ID NO: 649 - protein, 4 aa, Artificial Sequence
  synthetic peptide
  PEPTIDE (1)..(1): K, H, or R
  PEPTIDE (4)..(4): K, H, or R
XCCX

SEQ ID NO: 650 - protein, 5 aa, Artificial Sequence
  synthetic peptide
  PEPTIDE (1)..(1): K, H, or R
  misc_feature (3)..(3): Xaa can be any naturally occurring amino acid
  PEPTIDE (5)..(5): K, H, or R
XCXCX

SEQ ID NO: 651 - protein, 7 aa, Artificial Sequence
  synthetic peptide
  PEPTIDE (1)..(1): K, H, or R
  misc_feature (3)..(5): Xaa can be any naturally occurring amino acid
  PEPTIDE (7)..(7): K, H, or R
XCXXXCX

SEQ ID NO: 652 - protein, 4 aa, Artificial Sequence
  synthetic peptide
  PEPTIDE (1)..(1): K, H, or R
  PEPTIDE (3)..(3): K, H, or R
XCXC

SEQ ID NO: 653 - protein, 4 aa, Artificial Sequence
  synthetic peptide
  PEPTIDE (2)..(2): K, H, or R
  PEPTIDE (4)..(4): K, H, or R
CXCX

SEQ ID NO: 654 - protein, 6 aa, Artificial Sequence
  synthetic peptide
  PEPTIDE (1)..(1): K, H, or R
  misc_feature (3)..(4): Xaa can be any naturally occurring amino acid
  PEPTIDE (5)..(5): K, H, or R
XCXXXC

SEQ ID NO: 655 - protein, 6 aa, Artificial Sequence
  synthetic peptide
  PEPTIDE (1)..(1): K, H, or R
  misc_feature (3)..(3): Xaa can be any naturally occurring amino acid
  PEPTIDE (4)..(4): K, H, or R
  misc_feature (5)..(5): Xaa can be any naturally occurring amino acid
XCXXXC

SEQ ID NO: 656 - protein, 6 aa, Artificial Sequence
  synthetic peptide
  misc_feature (2)..(3): Xaa can be any naturally occurring amino acid
  PEPTIDE (4)..(4): K, H, or R
  PEPTIDE (6)..(6): K, H, or R
CXXXCX

SEQ ID NO: 657 - protein, 6 aa, Artificial Sequence
  synthetic peptide
  misc_feature (2)..(4): Xaa can be any naturally occurring amino acid
  misc_feature (6)..(6): Xaa can be any naturally occurring amino acid
CXXXCX

SEQ ID NO: 658 - protein, 6 aa, Artificial Sequence
  synthetic peptide
  PEPTIDE (2)..(2): K, H, or R
  misc_feature (3)..(4): Xaa can be any naturally occurring amino acid
  PEPTIDE (6)..(6): K, H, or R
CXXXCX

SEQ ID NO: 659 - protein, 5 aa, Artificial Sequence
  synthetic peptide
CGKHC

SEQ ID NO: 660 - protein, 5 aa, Artificial Sequence
  synthetic peptide
CGRHC

SEQ ID NO: 661 - protein, 5 aa, Artificial Sequence
  synthetic peptide
CGHHC

SEQ ID NO: 662 - protein, 5 aa, Artificial Sequence
  synthetic peptide
CGGHC

SEQ ID NO: 663 - protein, 5 aa, Artificial Sequence
  synthetic peptide
CGAHC

SEQ ID NO: 664 - protein, 5 aa, Artificial Sequence
  synthetic peptide
CGVHC

SEQ ID NO: 665 - protein, 5 aa, Artificial Sequence
  synthetic peptide
CGLHC

SEQ ID NO: 666 - protein, 5 aa, Artificial Sequence
  synthetic peptide
CGIHC

SEQ ID NO: 667 - protein, 5 aa, Artificial Sequence
  synthetic peptide
CGMHC

SEQ ID NO: 668 - protein, 5 aa, Artificial Sequence
  synthetic peptide
CGFHC

SEQ ID NO: 669 - protein, 5 aa, Artificial Sequence
  synthetic peptide
CGWHC

SEQ ID NO: 670 - protein, 5 aa, Artificial Sequence
  synthetic peptide
CGPHC

SEQ ID NO: 671 - protein, 5 aa, Artificial Sequence
  synthetic peptide
CGSHC

SEQ ID NO: 672 - protein, 5 aa, Artificial Sequence
  synthetic peptide
CGTHC

SEQ ID NO: 673 - protein, 5 aa, Artificial Sequence
  synthetic peptide
CGCHC

SEQ ID NO: 674 - protein, 5 aa, Artificial Sequence
  synthetic peptide
CGYHC

SEQ ID NO: 675 - protein, 5 aa, Artificial Sequence
  synthetic peptide
CGNHC

SEQ ID NO: 676 - protein, 5 aa, Artificial Sequence
  synthetic peptide
CGQHC

SEQ ID NO: 677 - protein, 5 aa, Artificial Sequence
  synthetic peptide
CGDHC

SEQ ID NO: 678 - protein, 5 aa, Artificial Sequence
  synthetic peptide
CGEHC